(19) United States
(12) Patent Application Publication
(10) Pub. No.: US 2024/0335525 A1
(43) Pub. Date: Oct. 10, 2024

(54) TRUNCATED INFLUENZA NEURAMINIDASE AND METHODS OF USING THE SAME

(71) Applicant: SANOFI PASTEUR INC., Swiftwater, PA (US)

(72) Inventors: Mario BARRO, Reading, MA (US); Yvonne CHAN, Cambridge, MA (US); Guadalupe CORTES-GARCIA, Brookline, MA (US); Raul GOMILA, Melrose, MA (US); Liqun HAN, Ayer, MA (US); Justin KLEKOTA, Sommerville, MA (US); Fuqin MA, Boston, MA (US); Satyajit RAY, Littleton, MA (US); Svetlana STEGALKINA, Pelham, NH (US); Tod STRUGNELL, Carlisle, MA (US); Irina USTYUGOVA, Hopkinton, MA (US); Thorsten VOGEL, Melrose, MA (US); Jianxin ZHANG, Acton, MA (US)

(21) Appl. No.: 18/682,372

(22) PCT Filed: Aug. 10, 2022
(86) PCT No.: PCT/US2022/039980

§ 371 Date: Feb. 8, 2024

Related U.S. Application Data
(60) Provisional application No. 63/231,790, filed on Aug. 11, 2021.

(51) Int. Cl. A61K 39/145; A61K 39/00; A61P 37/04; C07K 14/005; C07K 14/705; C12N 9/24

(52) U.S. Cl. CPC A61K 39/145; A61P 37/04; C07K 14/005; C07K 14/70596; C12N 9/2402; C12Y 302/01018; A61K 2039/645; C07K 2319/02; C12N 2760/16134

(57) ABSTRACT

Provided are modified influenza virus subtype 2 neuraminidase molecules lacking all or substantially all of the stalk region that form active, soluble tetrameric neuraminidase when expressed in host cells and vaccine compositions comprising the tetrameric neuraminidase or a nucleic acid encoding the modified monomeric influenza virus subtype 2 neuraminidase molecules that forms tetrameric NA when expressed in a cell. Also provided are methods of using the vaccine compositions to vaccinate or immunize a subject against influenza virus.

### CROSS REFERENCE TO RELATED APPLICATIONS

This application claims the benefit of, and relies on the filing date of, U.S. Provisional Patent Application No. 63/231,790, filed 11 Aug. 2021, the entire disclosures of which are herein incorporated by reference.

### BACKGROUND

Influenza is caused by a virus that attacks mainly the upper respiratory tract—the nose, throat and bronchi and rarely also the lungs. The infection usually lasts for about a week. It is characterized by sudden onset of high fever, myalgia, headache and severe malaise, non-productive cough, sore throat, and rhinitis. Most people recover within one to two weeks without requiring any medical treatment. However, in the very young, the elderly and people suffering from medical conditions, such as lung diseases, diabetes, cancer, kidney or heart problems, influenza poses a serious risk. In these people, the infection may lead to severe complications of underlying diseases, pneumonia, and death, although even healthy adults and older children can be affected as well. Annual seasonal influenza epidemics are thought to result in between three and five million cases of severe illness and between 250,000 and 500,000 deaths every year around the world.

Influenza virus is a member of the Orthomyxoviridae family. There are three main subtypes of influenza viruses, designated influenza A, influenza B, and influenza C. The influenza virion contains a segmented negative-sense RNA genome, which encodes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2). The HA, NA, M1, and M2 are membrane associated, whereas NP, PB1, PB2, PA, and NS2 are nucleocapsid associated proteins. The HA and NA proteins are envelope glycoproteins, primarily responsible for virus attachment and penetration of the viral particles into the cell and release from the cell, respectively.

Both HA and NA proteins are the sources of the major immunodominant epitopes for virus neutralization and protective immunity, making them important components for prophylactic influenza vaccines. The genetic makeup of influenza viruses allows frequent minor genetic changes, known as antigenic drift. Thus, the amino acid sequence of the major antigens of influenza, including HA and NA, is highly variable across certain groups, subtypes and/or strains. For this reason, current seasonal influenza vaccines are recommended every year and require yearly surveillance to account for mutations in HA and NA proteins (antigenic drift) and to match rapidly-evolving viral strains.

Influenza NA is a homotetrameric type II transmembrane glycoprotein, with each monomer having a globular head domain, a stalk region, a hydrophobic transmembrane domain, and a short, N-terminal cytoplasmic domain. Tetramerization of the head domain is important for formation of the enzymatic active site and the sialidase activity that is required for the release of new virus particles from infected cells. Sialidase activity also appears important for the virus to traverse mucus barriers in the host. The head domain is also the most immunologically relevant part of NA. Antibodies against the head region of influenza NA can block NA's enzymatic activity and interfere with viral pathogenesis, especially cell-to-cell spreading and transmission.

Recombinant NA or purified NA obtained by proteolysis or solubilization of viral membranes with detergents has been studied for structural, enzymatic, and immunological analysis. If a soluble version of NA is desired for use as an immunogen, then the NA molecule has to be expressed without the anchoring transmembrane region, which frequently results in the loss of stabilizing forces that help to hold the NA molecule in its tetrameric form. Without the transmembrane region, and the ability to embed the HA protein in the membrane, the stability of the tetrameric NA head is compromised, resulting in partial disassembly and loss of immunogenicity. Furthermore, the secondary and tertiary structure of the NA stalk and associated transmembrane domain is unknown, complicating rational protein engineering approaches based on native structure.

Thus, expression of soluble, tetrameric NA in cells is very challenging, particularly expression of soluble, tetrameric NA in high yield and/or in an appropriate host cell that is compatible with the large-scale production of a therapeutic, recombinant protein. These challenges stem, in part, from impaired assembly of tetrameric NA from recombinant NA constructs, resulting primarily in the expression of inactive, soluble monomers and dimers, or inclusion bodies (large aggregates of protein that pose major challenges for recovery of large-scale production of bioactive protein), and little to no expression of active, tetrameric NA. Mather et al., 1992, Virus Res., 26:127-39; Castrucci et al., J. Virology, 1993, 67(2):759-64; Martinet, Eur. J. Biochem., 1997, 247:332-38; Yongkiettrakul et al., 2011, J. Virological Methods, 156:44-51; Romanik et al., 2012, FEBS Journal, 279(Suppl. 1):52-576, 339. In one instance, a recombinant truncated NA was prepared that produced a mixed population of tetrameric NA, dimeric NA and monomeric NA when expressed in insect cells, however, this specific construct generated from the NA of the A/Victoria/3/1975 influenza strain was missing amino acids 1-45 of the NA protein (i.e., the cytoplasmic tail, the transmembrane region, and only the first several amino acids of the stalk region). DeRoo et al., Vaccine, 1996, 14(6):561-69. Further studies with the A/Victoria/3/1975 influenza strain used by DeRoo et al. showed that when substantially all of the stalk region was removed (i.e., a recombinant construct lacking amino acids 1-78 or 1-79 of the NA protein), only monomeric NA was produced when expressed in insect cells. Martinet, Eur. J. Biochem., 1997, 247:332-38. Viral NAs purified from viral membranes by proteolysis or detergent-solubilization are unstable and quickly lose enzymatic activity. Schmidt et al., PLos ONE, 2011, 6(2):e16284.

To overcome these challenges, the art teaches the use of a full-length, heterologous tetramerization domain fused to the NA head region to prepare soluble, recombinant NA that forms tetrameric NA. Schmidt et al., PLos ONE, 2011, 6(2):e16284; Da Silva et al., J Biol Chem, 2013, 288(1):644-53; Dai et al., 2016, J. Virology, 90(20):9457-70; Bosch et al., 2010, J. Virology, 84(19):10366-74; Prevato et al., 2015, PLos ONE, 10(8): e0135474. It has been shown that the structural role of the transmembrane and stalk domain in stabilizing the head region of NA can be approximated by using a full-length heterologous tetramerization domain that forms a four-helix bundle. This does not mean that the native stalk domain forms a four-helix bundle. If it did, then the structure would be easily predictable using bioinformatics algorithms, which it is not, and would have been demonstrated using x-ray crystallography and/or cryo-electron microscopy. While the full-length heterologous tetramerization domain provides a convenient recombinant tool for producing soluble, tetrameric NA, the use of a full-length heterologous tetramerization domain in a recombinant protein, has the potential to induce an immune response against the foreign tetramerization domain.

New recombinant influenza NA proteins (and nucleic acids encoding the same) are needed to help effectively control seasonal influenza infection and to avert pandemic outbreaks, particularly recombinant influenza NA proteins that can be expressed in cells to yield an immunogenic, tetrameric NA without the use of full-length, heterologous oligomerization sequences.

### SUMMARY

This application discloses modified monomeric influenza virus subtype 2 neuraminidase constructs lacking the cytoplasmic tail, the transmembrane region, and all or substantially all of the stalk region that form active, soluble tetrameric neuraminidase when expressed in host cells. The recombinant design strategy disclosed in this application allows for the large-scale production of recombinant tetrameric NA, permitting a rapid and flexible response to newly emerging variant influenza strains.

A first aspect is directed to a modified monomeric influenza virus subtype 2 neuraminidase (NA), comprising a head region of an influenza virus subtype 2 NA, wherein the cytoplasmic tail, transmembrane region and all or substantially all of the stalk region of the influenza virus subtype 2 NA have been removed from the modified monomeric influenza virus subtype 2 NA, wherein the modified monomeric influenza virus subtype 2 NA does not include a heterologous oligomerization domain, and wherein expression of the modified monomeric influenza virus subtype 2 NA in a cell results in the secretion of a tetrameric NA. In certain embodiments, the modified monomeric influenza virus subtype 2 neuraminidase comprises a signal peptide and the cytoplasmic tail, transmembrane region and all or substantially all of the stalk region of the influenza virus subtype 2 NA have been replaced by the signal peptide. The first aspect is also directed to an artificial nucleic acid that encodes the modified monomeric influenza virus subtype 2 NA. Various features and embodiments of this first aspect are described in further detail throughout the disclosure of this application.

A second aspect is directed to a tetrameric neuraminidase (NA), wherein the tetrameric NA comprises four copies of a modified monomeric influenza virus subtype 2 NA, wherein the modified monomeric influenza virus subtype 2 NA comprises a head region of an influenza virus subtype 2 NA but does not include 1) the cytoplasmic tail, transmembrane region and all or substantially all of the stalk region of the influenza virus subtype 2 NA and 2) a heterologous oligomerization domain. Various features and embodiments of this second aspect are described in further detail throughout the disclosure of this application.

A third aspect is directed to a vaccine composition comprising the tetrameric NA (second aspect) or an artificial nucleic acid molecule encoding the modified monomeric influenza virus subtype 2 NA (first aspect). The vaccine composition may also comprise an adjuvant. The vaccine composition may further comprise an influenza virus hemagglutinin. In certain embodiments, the influenza virus hemagglutinin is from a different, or mismatched, influenza strain than the modified influenza virus subtype 2 neuraminidase. Various features and embodiments of this third aspect are described in further detail throughout the disclosure of this application.

A fourth aspect is directed to an in vitro method of producing the tetrameric NA (second aspect), wherein the method comprises culturing host cells in a cell culture medium, wherein the host cells contain an artificial nucleic acid encoding the modified monomeric influenza virus subtype 2 NA (first aspect), and expressing the modified monomeric influenza virus subtype 2 NA in the host cells, wherein following expression of the modified monomeric influenza virus subtype 2 NA, the host cell supernatant contains the tetrameric NA (second aspect). The method may also further comprise a step of purifying the secreted tetrameric NA to yield a purified influenza virus subtype 2 tetrameric NA. Various features and embodiments of this fourth aspect are described in further detail throughout the disclosure of this application.

A fifth aspect is directed to a method of immunizing a subject against influenza virus comprising administering to the subject an immunologically effective amount of the vaccine composition (third aspect). Various features and embodiments of this fifth aspect are described in further detail throughout the disclosure of this application.

A sixth aspect is directed to a method of vaccinating a subject against influenza virus, the method comprising administering to the subject an amount of the vaccine composition (third aspect) effective to vaccinate the subject against influenza virus. Various features and embodiments of this sixth aspect are described in further detail throughout the disclosure of this application.

A seventh aspect is directed to other methods of using the vaccine composition (third aspect), including methods of inducing an immune response against an influenza virus NA, methods of preventing an influenza virus disease and methods of reducing one or more symptoms of influenza virus infection, such as a reduction in body weight or an increase in body temperature (fever). Various features and embodiments of this sixth aspect are described in further detail throughout the disclosure of this application.

The foregoing general summary and the following detailed description are exemplary and explanatory and are not restrictive of the claims.

### BRIEF DESCRIPTION OF THE DRAWING

The accompanying drawings, which are incorporated in and constitute a part of this specification, illustrate certain embodiments, and together with the written description, explain certain principles of the neuraminidase molecules, compositions, and methods disclosed herein.

FIG. 1 shows the schematic of a wild type (“WT”) subtype 2 influenza neuraminidase (“N2”), having a short, N-terminal cytoplasmic tail, a transmembrane region, a stalk region and a head region and a previously described, recombinant N2 in which the cytoplasmic tail, the transmembrane region, and substantially all of stalk region are replaced by a signal peptide (aka, secretion signal), a histidine-tag, and a full-length, heterologous tetrabrachion tetramerization domain (“tetNA”). FIG. 1 also includes a schematic of an engineered N2 in which the cytoplasmic tail and the transmembrane region were replaced by a signal peptide (aka, secretion signal) and an optional histidine-tag (“dTM36”); dTM36 contains the entire stalk and head region of N2. The last schematic in FIG. 1 is an exemplary engineered N2 in which the cytoplasmic tail, the transmembrane region, and substantially all of stalk region are replaced by a signal peptide (aka, secretion signal) and an optional histidine-tag (“dTM75”). The 6HIS TAG in FIG. 1 corresponds to SEQ ID NO: 135.

FIG. 2 shows the TAMIFLU® binding results and NA enzymatic activity of dTM75 variants from 37 different subtype 2 influenza strains expressed in cells following transfection. A tetrameric NA expressed from a modified A/Singapore/INFIMH-16-0019/2016 NA comprising a full-length, heterologous tetrabrachion tetramerization domain was used as a positive control.

FIG. 3A shows a neuraminidase sequence map of 100 subtype 2 influenza strains tested in a dTM75 high throughput screening assay, with clusters of strains showing higher NA sequence similarity relative to other clusters of strains.

FIG. 3B shows the clusters of strains arranged by the year in which the strain was isolated.

FIGS. 3C-D show the TAMIFLU® binding results (C) and NA enzymatic activity (D) from a high-throughput screen of dTM75 variants from 100 subtype 2 influenza strains. The top 37 dTM75 TAMIFLU® binders are shown.

FIG. 4 shows the TAMIFLU® binding results for a series of sequential stalk deletion variants from three strains: A/PERTH/16/2009 (PERT09), A/BELGIUM/4217/2015 (BELG15), and A/KANSAS/14/2017 (KANS17). The N2 sequential stalk deletion variants, include deletions of the neuraminidase stalk region starting from amino acid 60 and extending through amino acid 90 for PERT09, BELG15, and KANS17. Amino acids 59-94 (dTM60-dTM95) of PERT09 NA (SEQ ID NO: 65), BELG15 NA (SEQ ID NO: 5), and KANS17 NA (SEQ ID NO: 45) are shown in FIG. 4. The diameters of the circles show the amount of TAMIFLU® binding by the modified, stalk deletion variant NA molecules. FIG. 4 discloses SEQ ID NOS 136-138, respectively, in order of appearance.

FIG. 5 shows Size Exclusion Chromatography with Multi-Angle Light Scattering (SEC-MALS) analysis of NA protein purified following large-scale production of dTM75 constructs from PERT09, BELG15, KANS17, A/Peru/4617/2017 (PERU17) and A/Texas/71/2017 (TEX17). The top panel shows the percentage of tetrameric NA in the sample, while the bottom shows the molecular weight (MW) of the dTM75 NA in the sample, with monomeric dTM75 having a MW of about 60 kD and tetrameric dTM75 having a MW of about 210 kD, as discussed in Example 4. KANS17 dTM75 eluted in two peaks, pool 1 (monomer) and pool 2 (tetramer), with the other dTM75 constructs eluting in a single peak comprising tetrameric NA.

FIG. 6 shows the % tetrameric NA, thermostability, TAMIFLU® binding, and enzymatic activity of dTM75 variants and tet-NA constructs from PERT09, BELG15, KANS17, PERU17, and TEX17, following the large-scale production of the dTM75 variants and tet-NA constructs and purification.

FIG. 7 shows the purity, thermostability, enzymatic activity (MUNANA ratio of dTM to tet-NA), and TAMIFLU® binding (ratio of dTM to tet-NA) of a PERT09 tet-NA construct and PERT09 stalk truncated variants dTM75, dTM74, dTM73, dTM72, and dTM71, following the large-scale production and purification of the tet-NA construct and dTM variants.

FIG. 8A shows the dosing schedule for mice. Mice were immunized intramuscularly (IM) with a priming dose of the recombinant N2 protein (plus adjuvant) at day 0 followed by a boosting dose of the recombinant N2 protein (plus adjuvant) at day 21, and sera were collected at day 35, as described in Example 6.

FIG. 8B shows the immunogenicity of PERT09, BELG15, and KANS17 dTM75 and tet-NA proteins administered to mice as shown in FIG. 8A and described in Example 6. Mice were immunized with monomeric recombinant NA preparation from Pool 1 of KANS17 dTM75 or tetrameric recombinant NA protein preparations from PERT09 dTM75, BELG17 dTM75, tet-NA, or Pool 2 of KANS17 dTM75. NAI responses were measured against the H6N2 reassortant virus expressing homologous full-length NA by ELLA and expressed as IC50. The IgG specific-NA responses were measured against homologous tet-NA (as coating antigen) by ELISA and expressed as EC50 (50% maximal effective concentration). Individual animal titers were normalized (Log 2) and graphed as a box plot with the numbers representing the group mean titer. The dashed line indicates the assay's lower limit of detection.

FIG. 9A shows the dosing schedule for mice. Mice were immunized intramuscularly (IM) with a priming dose of the recombinant N2 protein (plus adjuvant) at day 0 followed by a boosting dose of the recombinant N2 protein (plus adjuvant) at day 21, and sera were collected at day 35, as described in Example 6.

FIG. 9B shows the immunogenicity of truncated PERT09 dTM75, dTM74, dTM73, dTM72, and dTM71 N2 proteins administered to mice as shown in FIG. 9A and described in Example 6. The IgG specific-NA responses were measured against homologous tet-NA by ELISA and expressed as EC50 (50% maximal effective concentration). NAI responses were measured against the H6N2 reassortant virus expressing homologous full-length NA by ELLA and expressed as IC50. Individual animal titers were normalized (Log 2) and graphed as a box plot with the numbers representing the group mean titer. The dashed line indicates the assay's lower limit of detection, while the shaded area represents titers within 4-fold of the tet-NA control protein.

FIG. 10A shows the dosing schedule for pre-immune ferrets. Ferrets were immunized intranasally with a priming dose of an influenza H1N2 reassortant virus expressing a wild type NA of interest at day 0 followed by a boosting dose of the unadjuvanted homologous recombinant NA (dTM75 or tet-NA), administered IM. Sera were collected at day 42, as described in Example 7.

FIG. 10B shows the immunogenicity of PERT09, BELG15, and KANS17 dTM75 and tet-NA proteins administered to pre-immune ferrets as described in Example 7. NAI responses were measured against the H6N2 reassortant virus expressing homologous full-length NA by ELLA and expressed as IC50. The IgG specific-NA responses were measured against homologous tet-NA by ELISA and expressed as EC50 (50% maximal effective concentration). Individual animal titers were normalized (Log 2) and graphed as a box plot with the numbers representing the group mean titer. The dashed line indicates the assay's lower limit of detection. “Mock” controls were groups that were only preimmunized with H1N2 viruses on day 0 but not boosted with a recombinant NA on day 21.

FIG. 11 shows the amino acid sequences for SEQ ID NO: 1-133.

FIG. 12A shows endpoints of a study (serological antibody response and protection against infection and/or disease severity) in naïve ferrets after immunization with 2 doses of truncated PERT09 dTM75 followed by challenge with PERT09 H3N2 influenza virus as described in Example 8.

FIG. 12B shows the dosing schedule for the naïve ferret study. Naïve ferrets were immunized intramuscularly (IM) with a priming dose of recombinant N2 protein (dTM75 or tet-NA) with and without adjuvant at day 0 followed by a boosting dose of recombinant N2 protein (dTM75 or tet-NA) with and without adjuvant at day 21 as described in Table 4 and Example 8. The naïve ferrets were then challenged intranasally with PERT09 H3N2 at day 43 as described in Example 8. Sera were collected at days 20, 42 and 57.

FIG. 13 shows the immunogenicity of PERT09 dTM75 and control proteins as indicated by NAI antibody titers in ferret sera following prime (day 20), boost (day 42) and challenge (day 57) as described in Example 8. NAI responses were measured against the H6N2 reassortant virus expressing homologous full-length NA by ELLA and expressed as 50% maximal inhibitory concentration (IC50) as described in Example 6 for mice. Individual animal titers are shown with the numbers representing the average titer for each group.

FIG. 14 shows the immunogenicity of PERT09 dTM75 and control proteins as indicated by NA-binding antibody titers (ELISA) in ferret sera following prime (day 20), boost (day 42) and challenge (day 57) as described in Example 8. The IgG specific-NA responses were measured against homologous tet-NA (as coating antigen) by ELISA and expressed as EC50 (50% maximal effective concentration) as described for the mice in Example 6. Individual animal titers are shown with the numbers representing the average titer for each group.

FIG. 15 shows a plot depicting viral shedding (AUC), and disease severity as indicated by peak body temperature change (degrees Celsius) and peak body weight change (percent change) following H3N2 influenza virus challenge in naïve ferrets previously immunized with 2 doses of PERT09 dTM75 as described in Example 8. Results from control groups are also shown.

FIG. 16 shows the reduction in total virus shedding measured as AUC (mean, range and mean-difference/p-value for pair-wise comparisons) after H3N2 influenza virus challenge in naïve ferrets previously immunized with 2 doses of PERT09 dTM75 as described in Example 8. Results from control groups are also shown.

FIG. 17 shows the reduction in peak body temperature change measured as change in body temperature over pre-challenge baseline (mean, range and mean-difference/p-value for pair-wise comparisons) after H3N2 influenza virus challenge in naïve ferrets previously immunized with 2 doses of PERT09 dTM75 as described in Example 8. Results from control groups are also shown.

FIG. 18 shows the decrease in peak body weight loss measured as % body weight change over pre-challenge baseline (mean, range and body weight % change distribution per treatment group) after H3N2 influenza virus challenge in naïve ferrets previously immunized with 2 doses of PERT09 dTM75 as described in Example 8. Results from control groups are also shown.

FIG. 19 shows the immunogenicity of PERT09 dTM75, SING16-rHA, and combinations of PERT09 dTM75 and SING16-rHA as indicated by NAI antibody titers in ferret sera following prime (day 20), boost (day 42) and challenge (day 57) as described in Example 9. NAI responses were measured against the H6N2 reassortant PERT09 virus expressing homologous full-length NA by ELLA and expressed as 50% maximal inhibitory concentration (IC50) as described in Example 6 for mice. Each symbol represents NAI titer of each individual ferret within the group (n=9). Bar graphs and a number within the bar represent Geometric Mean with 95% CI. The “ns” indicates not significantly different.

FIG. 20 shows the immunogenicity of PERT09 dTM75, SING16-rHA, and combinations of PERT09 dTM75 and SING16-rHA as indicated by NA-binding antibody titers (ELISA) in ferret sera following prime (day 20), boost (day 42) and challenge (day 57) as described in Example 9. The IgG specific-NA responses were measured against homologous PERT09 tet-NA (as coating antigen) by ELISA and expressed as EC50 (50% maximal effective concentration) as described for the mice in Example 6. Each symbol represents ELISA titer of each individual ferret within the group (n=9). Bar graphs and a number within the bar represent Geometric Mean with 95% CI. The “ns” indicates not significantly different.

FIG. 21 shows the immunogenicity of PERT09 dTM75, SING16-rHA, and combinations of PERT09 dTM75 and SING16-rHA as indicated by SING16 HAI titers in ferret sera following prime (day 20), boost (day 42) and challenge (day 57) as described in Example 9. Each symbol represents HAI titer of each individual ferret within the group (n=9). Bar graphs and a number within the bar represent Geometric Mean with 95% CI. The “ns” indicates not significantly different.

FIG. 22 shows the immunogenicity of PERT09 dTM75, SING16-rHA, and combinations of PERT09 dTM75 and SING16-rHA as indicated by PERT09 HAI titers in ferret sera following prime (day 20), boost (day 42) and challenge (day 57) as described in Example 9. Each symbol represents HAI titer of each individual ferret within the group (n=9). Bar graphs and a number within the bar represent Geometric Mean with 95% C1. The “ns” indicates not significantly different.

FIGS. 23A-C show the decrease in body weight following H3N2 influenza virus challenge in naïve ferrets previously immunized with 2 doses of PERT09 dTM75, 2 doses of SING16 rHA, or 2 doses of a combination of PERT09 dTM75 and SING16 rHA, as described in Example 9. Results from control groups are also shown. Lines demonstrate the average percent change in body weight overtime (14 days) for a given group that is indicated in the legend. Data for groups immunized with 5 μg+AF03 (FIG. 23A) or 45 μg (FIG. 23B) are split between two graphs, and PBS and PERT09 pre-infected control groups presented in each graph are derived from the same study. For peak of body weight loss comparison (FIG. 23C), a mean value of each group is depicted next to the bar graph, each symbol represents a weight loss value of each individual ferret within the group (n=9). Bar graphs and a number within the bar represent Mean and error bars represent SEM. Statistically significant differences between the groups are noted by * and were estimated using pairwise comparisons by ANOVA followed by Tukey test with p-values from 0.0001 to 0.005.

FIGS. 24A-C show plots depicting the intensity and duration of viral shedding following H3N2 influenza virus challenge in naïve ferrets previously immunized with 2 doses of PERT09 dTM75, 2 doses of SING16 rHA, or 2 doses of a combination of PERT09 dTM75 and SING16 rHA, as described in Example 9. Results from control groups are also shown. Lines demonstrate the mean of titers for a given group that is indicated in the legend. Data for groups immunized with 5 μg+AF03 (FIG. 24A) or 45 μg (FIG. 24B) are split between two graphs, and PBS and PERT09 pre-infected control groups presented in each graph are derived from the same study. FIG. 24C shows total viral shedding (AUC) and viral shedding at day 45.

FIG. 25A shows a time course identifying the number of ferrets with detectable virus at days 0-8 following H3N2 influenza virus challenge.

FIG. 25B shows the increase in peak body temperature change measured in the AM and PM as change in body temperature over pre-challenge baseline after H3N2 influenza virus challenge in naïve ferrets previously immunized with 2 doses of PERT09 dTM75, 2 doses of SING16 rHA, or 2 doses of a combination of PERT09 dTM75 and SING16 rHA, as described in Example 9. Results from control groups are also shown. For peak body temperature, a mean value of each group is presented on the bar graph, each symbol represents a peak value of each individual ferret within the group (n=9), a number within the bar represent mean and error bars are presented as SEM.

### DETAILED DESCRIPTION

### A. Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Antigen: As used herein, the term “antigen”, refers to an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include the truncated NA and tetrameric NA formed therefrom as described herein.

Approximately: As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Artificial nucleic acid molecule: As used herein, an artificial nucleic acid molecule may typically be understood to be a nucleic acid molecule, e.g. a DNA or an RNA, that does not occur naturally. In other words, an artificial nucleic acid molecule may be understood as a non-natural nucleic acid molecule. Such nucleic acid molecule may be non-natural due to its individual sequence (which does not occur naturally) and/or due to other modifications, e.g., structural modifications of nucleotides which do not occur naturally. An artificial nucleic acid molecule may be a DNA molecule, an RNA molecule or a hybrid-molecule comprising DNA and RNA portions. Typically, artificial nucleic acid molecules may be designed and/or generated by genetic engineering methods to correspond to a desired artificial sequence of nucleotides (heterologous sequence). Further, the term “artificial nucleic acid molecule” is not restricted to mean “one single molecule” but is, typically, understood to comprise an ensemble of identical molecules. Accordingly, it may relate to a plurality of identical molecules contained in an aliquot.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Epitope: As used herein, the term “epitope” includes any moiety that is specifically recognized by an immunoglobulin (e.g., antibody or receptor) binding component in whole or in part. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Glycosylation: As used herein, refers to the addition of a saccharide unit to a protein. “N-glycan,” as used herein, refers to a saccharide chain attached to a protein at the amide nitrogen of an N (asparagine) residue of the protein. As such, an N-glycan is formed by the process of N-glycosylation. This glycan may be a polysaccharide.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to render a subject protected from an infectious disease.

Immunologically effective amount: As used herein, the term “immunologically effective amount” means an amount sufficient to immunize a subject.

In some embodiments: As used herein, the term “in some embodiments” refers to embodiments of all aspects of the disclosure, unless the context clearly indicates otherwise.

Monomeric influenza virus neuraminidase: Wild-type influenza virus neuraminidase (NA) is tetramer of four identical monomers. Each NA monomer in the wild-type influenza NA consists of four distinct structural domains: the enzymatic head region, the stalk region, the transmembrane region, and the cytoplasmic tail. As used herein, the term “monomeric influenza virus neuraminidase” refers to a NA monomer that can combine with three other NA monomers to form tetrameric NA. In some embodiments, the modified monomeric influenza virus neuraminidase described herein lacks the cytoplasmic tail, the transmembrane domain and all or substantially all of the stalk domain.

N: As used herein, “N1” refers to an influenza virus subtype 1 neuraminidase (NA). Type A influenza viruses are divided into Groups 1 and 2. Groups 1 and 2 are further divided into subtypes, which refers to classification of a virus based on the sequences of two proteins on the surface of the virus hemagglutinin (HA) and NA. Currently, there are 18 recognized HA subtypes (H1-H118) and 11 recognized NA subtypes (N1-N11). N1 is thus distinct from the other NA subtypes, N2-N11

N2: As used herein, “N2” refers to an influenza virus subtype 2 neuraminidase (NA). Type A influenza viruses are divided into Groups 1 and 2. Groups 1 and 2 are further divided into subtypes, which refers to classification of a virus based on the sequences of two proteins on the surface of the virus hemagglutinin (HA) and NA. Currently, there are 18 recognized HA subtypes (H1-H18) and 11 recognized NA subtypes (N1-N11). N2 is thus distinct from the other NA subtypes, N1 and N3-N11.

NB: As used herein, “NB” refers to an influenza B neuraminidase (NA). Influenza B strains are classified into two lineages: B/Yamagata and B/Victoria.

Pandemic strain: A “pandemic” influenza strain is one that has caused or has capacity to cause pandemic infection of subject populations, such as human populations. In some embodiments, a pandemic strain has caused pandemic infection. In some embodiments, such pandemic infection involves epidemic infection across multiple territories; in some embodiments, pandemic infection involves infection across territories that are separated from one another (e.g., by mountains, bodies of water, as part of distinct continents, etc.) such that infections ordinarily do not pass between them.

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Seasonal strain: A “seasonal” influenza strain is one that has caused or has capacity to cause a seasonal infection (e.g., annual epidemic) of subject populations, such as human populations. In some embodiments, a seasonal strain has caused seasonal infection.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. “Sequence identity” between two nucleic acid sequences indicates the percentage of nucleotides that are identical between the sequences. “Sequence identity” between two amino acid sequences indicates the percentage of amino acids that are identical between the sequences. Homologs or variants of a given gene or protein will possess a relatively high degree of sequence identity when aligned using standard methods.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional and/or structural property of said given sequence, e.g., and in some instances, are functionally and/or structurally equivalent to said given sequence. In some embodiments, a nucleic acid sequence or amino acid sequence having a particular degree of identity to a given nucleic acid sequence or amino acid sequence is functionally and/or structurally equivalent to said given sequence.

Stalk region: As used herein the “stalk region” of influenza subtype 2 neuraminidase refers to a region of about amino acid 36 to about amino acid 82 of the subtype 2 neuraminidase. As used herein “substantially all of a stalk region” refers to amino acid 36 to amino acid 69 of the stalk region of an influenza virus subtype 2 NA. Thus, a modified N2 lacking the cytoplasmic tail, the transmembrane region, and substantially all of the stalk region may lack amino acids 1-70, 1-71, 1-72, 1-73, 1-74, 1-75, 1-76, 1-77, 1-78, 1-79, 1-80, or 1-81 of an influenza virus subtype 2 NA. Put another way, the modified N2 described herein can include up to 13 of the most C-terminal amino acids of the stalk region of the influenza virus subtype 2 NA, where the most C-terminal amino acids of the stalk region typically refer to amino acids 70-82 of the N2. In certain embodiments, the cytoplasmic tail, the transmembrane region, and the entire stalk region (e.g., amino acids 1-82) have been removed from the modified N2.

Standard of Care Strain: Each year, based on intensive surveillance efforts, the World Health Organization (WHO) selects influenza strains to be included in the seasonal vaccine preparations. As used herein, the term “standard of care strain” or “SOC strain” refers to an influenza strain that is selected by the World Health Organization (WHO) to be included in the seasonal vaccine preparations. A standard of care strain can include a historical standard of care strain, a current standard of care strain or a future standard of care strain.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Tetramerization domain: As used herein, the term “tetramerization domain” refers to an amino acid sequence encoding a domain that causes the tetrameric assembly of a polypeptide or protein. A tetramerization domain that is not native to a particular protein may be termed an artificial or a heterologous tetramerization domain. Exemplary tetramerization domains include, but are not limited to, sequences from Tetrabrachion, GCN4 leucine zippers, or vasodilator-stimulated phosphoprotein (VASP).

Vaccination: As used herein, the term “vaccination” or “vaccinate” refers to the administration of a composition to generate an immune response, for example to a disease-causing agent such as an influenza virus. Vaccination can be administered before, during, and/or after exposure to a disease-causing agent, and/or to the development of one or more symptoms, and in some embodiments, before, during, and/or shortly after exposure to the agent. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response. In some embodiments, vaccination includes multiple administrations, appropriately spaced in time, of a vaccinating composition.

Wild type (WT): As is understood in the art, the term “wild type” generally refers to a normal form of a protein or nucleic acid, as is found in nature. For example, wild type NA polypeptides are found in natural isolates of influenza virus. A variety of different wild type NA sequences can be found in the NCBI influenza virus sequence database.

### B. Truncated N2

This application discloses recombinant influenza subtype 2 neuraminidase (N2) lacking the cytoplasmic tail, the transmembrane region and all or substantially all of the stalk region that forms soluble, tetrameric neuraminidase (NA) when expressed in cells without the use of a heterologous tetramerization domain.

### 1. Nomenclature for Influenza Virus

All nomenclature used to classify influenza virus is that commonly used by those skilled in the art. Thus, a Type, or Group, of influenza virus refers to the three main types of influenza: influenza Type A, influenza Type B or influenza Type C that infect humans. Influenza A and B cause significant morbidity and mortality each year. It is understood by those skilled in the art that the designation of a virus as a specific Type relates to sequence difference in the respective Ml (matrix) protein or P (nucleoprotein). Type A influenza viruses are further divided into group 1 and group 2. These groups are further divided into subtypes, which refers to classification of a virus based on the sequences of two proteins on the surface of the virus HA and NA. Currently, there are 18 recognized HA subtypes (H1-H18) and 1 recognized NA subtypes (N1-N11). Group 1 contains N1, N4, N5, and N8 and H1, H2, H5, H6, H8, H9, H11, H12, H13, H16, H17 and H18. Group 2 contains N2, N3, N6, N7, and N9 and H3, H4, H7, H10, H14, and H15. N10 and N11 have been identified in influenza-like genomes isolated from bats. Wu et al., Trends in Microbiology, 2014, 22(4):183-91. While there are potentially 198 different influenza A subtype combinations, only about 131 subtypes have been detected in nature. Current subtypes of influenza A viruses that commonly circulate in the human population, giving rise to seasonal outbreaks, include: A(H1N1) and A(H3N2). Influenza A subtypes can be further broken down into different genetic “clades” and “sub-clades.” Finally, the term strain refers to viruses within a subtype that differ from one another in that they have small, genetic variations in their genome.

For convenience, certain abbreviations can be used to refer to protein constructs, and portions thereof, described herein. For example, NA can refer to influenza neuraminidase protein, or a portion thereof. N2 refers to neuraminidase from an influenza subtype 2 strain. The term tet-NA refers to a recombinant NA comprising a heterologous tetramerization domain that forms tetrameric NA when expressed in cells. HA refers to hemagglutinin or a portion thereof.

### 2. Neuraminidase (NA)

Neuraminidase (NA), along with hemagglutinin (HA), is one of the two major influenza surface proteins. The functions of both NA and HA involve interactions with sialic acid, a terminal molecule bound to sugar moieties on glycoproteins or glycolipids expressed on the surface of cells. The binding of HA to sialic acid on the cell surface induces endocytosis of the virus by the cell, allowing the virus to gain entry and infect cells. Sialic acid is also added to HA and NA as part of the glycosylation process that occurs within infected cells. NA removes sialic acid from cellular glycoproteins and glycolipids and from newly synthesized HA and NA on nascent virions. The removal of sialic acid by NA promotes the efficient release of viral particles from the surface of infected cells by preventing aggregation of viral particles. It also prevents virus from binding via HA to dying cells that have already been infected, promoting the further spread of the viral infection.

a. Structure of Wild Type Neuraminidase

NA is a type II transmembrane glycoprotein that assembles on the virus surface as a tetramer of four identical monomers. The molecular mass of the wild type monomer is about 55-72 kDa, depending on the influenza subtype; the molecular mass of the tetramer is about 240-260 kDa, depending on the influenza subtype. Each NA monomer consists of four distinct structural domains: the enzymatic head region, the stalk region, the transmembrane region, and the cytoplasmic tail. The largest domain is the head region, which is tethered to the viral membrane by a stalk region connected to the transmembrane region and finally the N-terminal cytoplasmic tail. Techniques known to one of skill in the art may be used to delineate the different domains of an influenza virus neuraminidase.

The stalk region among different influenza A virus subtypes, including N1 and N2, can vary significantly in size and amino acid structure. Blok et al., Biochemistry, 1982, 21:4001-4007. The differences in stalk length are thought to regulate the distance of the enzymatic head region and impact the ability of NA to access sialic acid on cell surface receptors, with shorter stalk regions correlating with reduced sialidase activity. Da Silva et al., J Biol Chem, 2013, 288(1):644-53; McAuley et al., Frontiers in Microbiology, 2019, 10(39). Notwithstanding the variability among stalk regions of different subtypes, NA stalk regions also share some structural features, including at least one cysteine residue and a potential glycosylation site. The cysteine residue(s) may be involved in the formation of disulfide bonds between NA monomers and assist in the formation of a stabilized NA tetramer, while the glycosylation site may contribute to tetramer stabilization. McAuley et al., Frontiers in Microbiology, 2019, 10(39). For example, a conserved cysteine residue at or around amino acid position 78 of N2 is believed to play a role in the tetramer assembly mechanism. Shtyrya et al., Acta Naturae. 2009; 1(2): 26-32.

The enzymatic head region is comprised of four monomers. Each monomer in the head forms a conserved six-bladed propeller structure. Each blade has four anti-parallel β-sheets that are stabilized by disulfide bonds and connected by loops of varying length. McAuley et al., Frontiers in Microbiology, 2019, 10(39). Tetramerization of the monomers is important for the formation of the active site and synthesis of the enzymatically active NA. Dai et al., J. Virology, 2016, 90(20):9457-70.

b. Subtype 2 (N2) Influenza Virus Neuraminidase

Although the amino acid sequence and length of NA can vary significantly between different influenza A virus NA subtypes, such as N1 and N2, and particularly the NA stalk regions of different influenza A virus NA subtypes, the amino acid sequence length of N2 from different influenza strains is typically about 469 amino acids, with a few strains having about one or two (or more) amino acid insertions or deletions, typically in the head region. When referring to specific amino acid residues in a wild type N2, the specific amino acid residue numbers are based on N2 numbering, as understood in the art. The N-terminal cytoplasmic tail typically corresponds to amino acid 1-6 of the wild type N2 sequence, while the transmembrane region typically corresponds to amino acids 7-35 of the wild type N2 sequence. For example, in the wild type NA sequence of the strain A/PERTH/16/2009 (SEQ ID NO: 65), the cytoplasmic tail corresponds to amino acids 1-6 of SEQ ID NO: 65, while the transmembrane region corresponds to amino acids 7-35 of SEQ ID NO: 65. The length of the N2 stalk region is typically about 46 amino acids in length, starting at about amino acid 36 and ending at about amino acid 82 of the wild type N2 sequence. For example, in the wild type NA sequence of the strain A/PERTH/16/2009 (SEQ ID NO: 65), the stalk region corresponds to amino acid 36 to about amino acid 82 of SEQ ID NO: 65. However, the precise boundary between the end of the N2 stalk region and the start of the N2 head region has not been resolved by x-ray crystallography.

### 3. Removal of all or Substantially all of the Stalk Region

A recombinant N2 was designed that lacks the cytoplasmic tail, the transmembrane region and all or substantially all of the stalk region. The recombinant NA does not contain a heterologous tetramerization domain, which the art teaches is important for producing soluble, recombinant NA proteins. Schmidt et al., PLos ONE, 2011, 6(2):e16284; Da Silva et al., J Biol Chem, 2013, 288(1):644-53; Dai et al., 2016, J. Virology, 90(20):9457-70; Bosch et al., 2010, J. Virology, 84(19):10366-74. Yet when expressed in cells, it was surprisingly discovered that the truncated N2, lacking all or substantially all of the stalk region, formed soluble tetrameric NA.

Initial experiments showed that recombinant N2 lacking amino acids 1-74 formed tetrameric NA when expressed in cells. This property was observed in truncated N2 sequences from different strains. When the deletion analysis was expanded to cover deletion variants having less and more of the stalk region removed, it was discovered that recombinant N2 lacking amino acids 1 to at least amino acid 70-82 formed tetrameric NA when expressed in cells. In fact, in certain instances, it was discovered that the N-terminal deletion could encompass the entire stalk region and extend several amino acids into the head region and still form soluble, tetrameric NA.

One aspect of this disclosure is directed to an artificial nucleic acid molecule encoding a modified monomeric influenza virus subtype 2 neuraminidase (N2). In certain embodiments, the modified monomeric N2 lacks the cytoplasmic tail, transmembrane region and all or substantially all of the stalk region and comprises a head region of an N2, wherein the modified monomeric influenza virus subtype 2 NA does not include a heterologous oligomerization domain, and wherein expression of the modified monomeric N2 in a cell results in the secretion of a tetrameric NA. In certain embodiments, the modified monomeric N2 comprises a signal peptide and a head region of an N2, wherein a cytoplasmic tail region, a transmembrane region and all or substantially all of a stalk region of the N2 have been replaced by the signal peptide, wherein the modified monomeric N2 does not include a heterologous oligomerization domain. Expression of the modified monomeric N2 in a cell results in the secretion of a tetrameric NA.

In some embodiments, the artificial nucleic acid molecule encodes a modified monomeric N2, wherein amino acid 1 to at least amino acid 70-82 of an N2 (e.g., SEQ ID NO: 1-115) have been replaced by the signal peptide.

In some embodiments, the artificial nucleic acid molecule encodes a modified monomeric N2, wherein amino acids 1-70 of an N2 (e.g., SEQ ID NO: 1-115) have been replaced by the signal peptide. Such embodiments are also referred to as dTM71.

In some embodiments, the artificial nucleic acid molecule encodes a modified monomeric N2, wherein amino acids 1-71 of an N2 (e.g., SEQ ID NO: 1-115) have been replaced by the signal peptide in the modified monomeric N2. Such embodiments are also referred to as dTM72.

In some embodiments, the artificial nucleic acid molecule encodes a modified monomeric N2, wherein amino acids 1-72 of an N2 (e.g., SEQ ID NO: 1-115) have been replaced by the signal peptide in the modified monomeric N2. Such embodiments are also referred to as dTM73.

In some embodiments, the artificial nucleic acid molecule encodes a modified monomeric N2, wherein amino acids 1-73 of an N2 (e.g., SEQ ID NO: 1-115) have been replaced by the signal peptide in the modified monomeric N2. Such embodiments are also referred to as dTM74.

In some embodiments, the artificial nucleic acid molecule encodes a modified monomeric N2, wherein amino acids 1-74 of an N2 (e.g., SEQ ID NO: 1-115) have been replaced by the signal peptide in the modified monomeric N2. Such embodiments are also referred to as dTM75.

In some embodiments, the artificial nucleic acid molecule encodes a modified monomeric N2, wherein amino acids 1-75 of an N2 (e.g., SEQ ID NO: 1-115) have been replaced by the signal peptide in the modified monomeric N2. Such embodiments are also referred to as dTM76.

In some embodiments, the artificial nucleic acid molecule encodes a modified monomeric N2, wherein amino acids 1-76 of an N2 (e.g., SEQ ID NO: 1-115) have been replaced by the signal peptide in the modified monomeric N2. Such embodiments are also referred to as dTM77.

In some embodiments, the artificial nucleic acid molecule encodes a modified monomeric N2, wherein amino acids 1-77 of an N2 (e.g., SEQ ID NO: 1-115) have been replaced by the signal peptide in the modified monomeric N2. Such embodiments are also referred to as dTM78.

In some embodiments, the artificial nucleic acid molecule encodes a modified monomeric N2, wherein amino acids 1-78 of an N2 (e.g., SEQ ID NO: 1-115) have been replaced by the signal peptide in the modified monomeric N2. Such embodiments are also referred to as dTM79.

In some embodiments, the artificial nucleic acid molecule encodes a modified monomeric N2, wherein amino acids 1-79 of an N2 (e.g., SEQ ID NO: 1-115) have been replaced by the signal peptide in the modified monomeric N2. Such embodiments are also referred to as dTM80.

In some embodiments, the artificial nucleic acid molecule encodes a modified monomeric N2, wherein amino acids 1-80 of an N2 (e.g., SEQ ID NO: 1-115) have been replaced by the signal peptide in the modified monomeric N2. Such embodiments are also referred to as dTM81.

In some embodiments, the artificial nucleic acid molecule encodes a modified monomeric N2, wherein amino acids 1-81 of an N2 (e.g., SEQ ID NO: 1-115) have been replaced by the signal peptide in the modified monomeric N2. Such embodiments are also referred to as dTM82.

In some embodiments, the artificial nucleic acid molecule encodes a modified monomeric N2, wherein amino acids 1-82 of an N2 (e.g., SEQ ID NO: 1-115) have been replaced by the signal peptide in the modified monomeric N2. Such embodiments are also referred to as dTM83.

One aspect of this disclosure is directed to a tetrameric NA, comprising four copies of a modified monomeric N2. As discussed above, the signal peptide is normally cleaved during post-translational processing such that the secreted, polypeptide typically does not contain the signal peptide. Thus, the modified monomeric N2 that is part of the tetrameric NA typically comprises only a head region of an influenza virus subtype 2 NA and does not include 1) a cytoplasmic tail region, a transmembrane region and all or substantially all of a stalk region of the influenza virus subtype 2 NA, and 2) a heterologous oligomerization domain.

In some embodiments, the modified monomeric N2 lacks all of the stalk region of the N2 or lacks amino acid 1 through at least amino acid 70-82 of the N2.

In some embodiments, the modified monomeric N2 lacks amino acids 1-70 of the N2 (e.g., SEQ ID NO: 1-115). Such embodiments, as well as tetrameric NA formed by this modified monomeric N2, are also referred to as dTM71.

In some embodiments, the modified monomeric N2 lacks amino acids 1-71 of N2 (e.g., SEQ ID NO: 1-115). Such embodiments, as well as tetrameric NA formed by this modified monomeric N2, are also referred to as dTM72.

In some embodiments, the modified monomeric N2 lacks amino acids 1-72 of the N2 (e.g., SEQ ID NO: 1-115). Such embodiments, as well as tetrameric NA formed by this modified monomeric N2, are also referred to as dTM73.

In some embodiments, the modified monomeric N2 lacks amino acids 1-73 of the N2 (e.g., SEQ ID NO: 1-115). Such embodiments, as well as tetrameric NA formed by this modified monomeric N2, are also referred to as dTM74.

In some embodiments, the modified monomeric N2 lacks amino acids 1-74 of the N2 (e.g., SEQ ID NO: 1-115). Such embodiments, as well as tetrameric NA formed by this modified monomeric N2, are also referred to as dTM75.

In some embodiments, the modified monomeric N2 lacks amino acids 1-75 of the N2 (e.g., SEQ ID NO: 1-115). Such embodiments, as well as tetrameric NA formed by this modified monomeric N2, are also referred to as dTM76.

In some embodiments, the modified monomeric N2 lacks amino acids 1-76 of the N2 (e.g., SEQ ID NO: 1-115). Such embodiments, as well as tetrameric NA formed by this modified monomeric N2, are also referred to as dTM77.

In some embodiments, the modified monomeric N2 lacks amino acids 1-77 of the N2 (e.g., SEQ ID NO: 1-115). Such embodiments, as well as tetrameric NA formed by this modified monomeric N2, are also referred to as dTM78.

In some embodiments, the modified monomeric N2 lacks amino acids 1-78 of the N2 (e.g., SEQ ID NO: 1-115). Such embodiments, as well as tetrameric NA formed by this modified monomeric N2, are also referred to as dTM79.

In some embodiments, the modified monomeric N2 lacks amino acids 1-79 of the N2 (e.g., SEQ ID NO: 1-115). Such embodiments, as well as tetrameric NA formed by this modified monomeric N2, are also referred to as dTM80.

In some embodiments, the modified monomeric N2 lacks amino acids 1-80 of the N2 (e.g., SEQ ID NO: 1-115). Such embodiments, as well as tetrameric NA formed by this modified monomeric N2, are also referred to as dTM81.

In some embodiments, the modified monomeric N2 lacks amino acids 1-81 of the N2 (e.g., SEQ ID NO: 1-115). Such embodiments, as well as tetrameric NA formed by this modified monomeric N2, are also referred to as dTM82.

In some embodiments, the modified monomeric N2 lacks amino acids 1-82 of the N2 (e.g., SEQ ID NO: 1-115). Such embodiments, as well as tetrameric NA formed by this modified monomeric N2, are also referred to as dTM83.

Typically, in the modified monomeric N2, the head region of the N2 comprises the full-length head region of the wild type N2, including, for example amino acid 83 to the terminal amino acid of the wild type N2, which for most N2 strains is amino acid 469, with a few strains having about one or two (or more) amino acid insertions or deletions, typically in the head region. In some embodiments, the N-terminal NA truncation can extend past the stalk region into the head region, as long as the modified monomeric N2 is still capable of forming a tetrameric NA when expressed in a cell. For example, as shown in the examples, modified monomeric N2 in which all of the stalk region and several amino acids at the start of the head region have been deleted can form tetrameric NA when expressed in cells. In some embodiments, the modified monomeric N2 lacks the cytoplasmic tail region, the transmembrane region, all of the stalk region of the N2 (e.g., SEQ ID NO: 1-115) and the first 1-5 amino acids of the head region or the artificial nucleic acid molecule encodes such a modified monomeric N2. In some embodiments, the modified monomeric N2 lacks amino acid 1 to the first or second amino acid of the head region or the artificial nucleic acid molecule encodes such a modified monomeric N2. For example, the modified monomeric N2 can lack amino acids 1-83 of the influenza virus subtype 2 NA (e.g., SEQ ID NO: 1-115) or the artificial nucleic acid molecule encodes such a modified monomeric N2 (also referred to as dTM84). For example, the modified monomeric N2 can lack amino acids 1-84 of the influenza virus subtype 2 NA (e.g., SEQ ID NO: 1-115) or the artificial nucleic acid molecule encodes such a modified monomeric N2 (also referred to as dTM85).

As noted above, the modified monomeric N2 can lack amino acids 1-74 of the N2 (e.g., SEQ ID NO: 1-115). Such embodiments, as well as tetrameric NA formed by this modified monomeric N2, are also referred to as dTM75. Representative dTM75 constructs include, but are not limited to, any one of SEQ ID NO: 117-131.

### 4. Signal Peptide

The wild type NA protein is a membrane bound protein that includes a transmembrane domain. To make a soluble NA protein, it is possible to delete the transmembrane domain and add a signal peptide. The signal peptide targets the recombinant NA protein to the secretory pathway so that the recombinant NA protein is secreted from the host cell in which the recombinant NA is expressed. When the modified monomeric N2 nucleic acid is translated into a polypeptide inside the host cell, the polypeptide contains the signal peptide. However, during post-translational processing, the signal peptide is cleaved, such that the secreted polypeptide no longer contains the signal peptide. As such, although the nucleic acid constructs described herein can encode a signal peptide to target the modified monomeric N2 to the secretory pathway, the soluble tetrameric NA obtained from host cells that express the nucleic acid constructs are made up of four modified N2 monomers that no longer contain the signal peptide.

Any signal peptide or nucleic acid encoding the same can be used in the recombinant NA constructs. The signal peptide should be a signal peptide sequence that is recognized by the host cell that is used to express the recombinant N2. The signal peptide may be a heterologous signal peptide from a source other than an influenza virus or it may be a signal peptide from an influenza virus, such as the signal peptide for hemagglutinin. In some embodiments, the signal peptide is a mammalian signal peptide. In some embodiments, the signal peptide is a human signal peptide, including, for example, the signal peptide is from CD5, immunoglobulin kappa light chain, serum albumin, azurocidin, trypsinogen, interleukin-2, and prolactin or a derivative thereof that directs the secretion of the modified NA. Kober et al., Biotechnology & Bioengineering, 2012, 110(4):1164-63; Dalton et al., Protein Sci., 2014, 23(5):517-25. In some embodiments, the signal peptide is a CD5 signal peptide. In some embodiments, the CD5 signal peptide comprises the amino acid sequence MPMGSLQPLATLYLLGMLVASVLS (SEQ ID NO: 132) or MPMGSLQPLATLYLLGMLVASCLG (SEQ ID NO: 133).

### 5. Linker Sequence

In some embodiments, the modified monomeric N2 may comprise an optional linker sequence that links the signal peptide to the truncated NA. In this way, the linker sequence can help to separate the head region from the remainder of the modified N2. For example, in some embodiments, the linker sequence can link the signal peptide to the head region in embodiments where the entire stalk region has been deleted. In other embodiments, the linker sequence can link the signal peptide to the stalk region in embodiments where substantially all of the stalk region has been deleted. If the modified NA contains a protein tag sequence, the linker sequence can be inserted on the N-terminus and/or C-terminus of the protein tag sequence. The linker sequence is not part of the naturally occurring NA amino acid sequence. In some embodiments, the linker sequence comprises glycine and/or serine residues and optionally one or more alanine residues. In some embodiments, the linker sequence is about 2-10 amino acids in length, including 2, 3, 4, 5, 6, 7, 8, 9, or 10 amino acids in length and help provide flexibility between different regions or portions of the recombinant NA. In some embodiments, the linker sequence comprises or consists of the amino acid sequence GSG, AGS, or AGSG (SEQ ID NO: 134). In some embodiments, the linker sequence comprises or consists of the amino acid sequence GS or SG.

Any other linker sequence known in the art can be used in the recombinant NA constructs. Other exemplary peptide linkers are those described in U.S. Pat. Nos. 4,751,180, 4,935,233, and 5,073,627, each of which is hereby incorporated by reference in its entirety. Using conventional techniques, a DNA sequence encoding a desired linker sequence may be inserted between, and in the same reading frame as, for example, DNA sequences encoding the signal peptide and the head region, in embodiments where the entire stalk region has been deleted, or between the terminal amino acid of the stalk region and the head region, in embodiments where substantially all of the stalk region has been deleted.

### 6. Protein Tag Sequence

The modified monomeric N2 may comprise an optional protein tag sequence that can be used to purify or otherwise identify the modified N2. For example, in some embodiments, the modified N2 comprises a histidine tag sequence. Any protein tag sequence known in the art can be used in the recombinant NA constructs.

### 7. Protease Cleavage Site

The modified monomeric NA may also comprise an optional protease cleavage site. The protease cleavage site can be used to modify the recombinant NA after the recombinant NA is expressed in a host cell by promoting the removal of sequences (e.g., protein tag sequence, linker sequence, etc.) from the final soluble NA protein. Any protease cleavage site known in the art can be used in the recombinant NA constructs.

### 8. Tetrameric NA

It has been discovered that when a nucleic acid encoding the modified monomeric N2, as described herein, is expressed in cells, soluble tetrameric NA is detectable in the cell supernatant and can be purified therefrom in high yields. Although not all N2 strains produced soluble tetrameric NA in detectable amounts, the majority of N2 strains tested produced detectable amounts of soluble tetrameric NA, showing that this truncated stalk design strategy can be broadly applied to the NA protein from various N2 influenza strains. Certain N2 strains and certain stalk-deleted variants of specific strains produce higher yields of soluble, tetrameric NA when expressed in cells. As shown in the examples, and discussed in further detail below, high throughput screening can be used to easily identify those strains that produce soluble tetrameric NA, as well as to quantify the amount of soluble tetrameric NA produced. The same high throughput screening can also be used to test modified NA stalk truncated variants with varying lengths of the stalk region to identify the variants producing soluble tetrameric NA or the highest amount of soluble, tetrameric NA, as demonstrated in the examples.

Most of the soluble tetrameric NA formed from the modified monomeric N2 constructs described herein retain neuraminidase enzymatic activity. In some embodiments, the soluble tetrameric NA described herein has neuraminidase activity. Neuraminidase activity can be measured using techniques known in the art, including, for example, a MUNANA assay or an NA-STAR® assay (ThermoFisher Scientific, Waltham, MA). In the MUNANA assay, 2′-(4-methylumbelliferyl)-alpha-D-N-acetylneuraminic acid (MUNANA) is used as a substrate. Any enzymatically active neuraminidase contained in the sample cleaves the MUNANA substrate, releasing 4-Methylumbelliferone (4-MU), a fluorescent compound. Thus, the amount of neuraminidase activity in a test sample correlates with the amount of 4-MU released. In some embodiments, the soluble tetrameric NA, as described herein, has an activity ranging from 1 to 25 mole/min/μg, as measured by a MUNANA assay.

For purposes of determining the neuraminidase activity of a soluble tetrameric NA of the present disclosure, a MUNANA assay should be performed using the following conditions: mix soluble tetrameric NA with buffer [33.3 mM 2-(N-morpholino) ethanesulfonic acid (MES, pH 6.5), 4 mM CaCl2), 50 mM BSA] and substrate (100 μM MUNANA) and incubate for 1 hour at 37° C. with shaking; stop the reaction by adding an alkaline pH solution (0.2M Na₂CO₃); measure fluorescence intensity, using excitation and emission wavelengths of 355 and 460 nm, respectively; and calculate enzymatic activity against a 4MU reference. If necessary, an equivalent assay can be used to measure neuraminidase enzymatic activity.

To detect tetrameric NA, a new TAMIFLU®-binding assay was developed, as described in the examples. The TAMIFLU® screening assay is also described in further detail in U.S. Application No. 63/231,795, entitled METHODS AND RELATED ASPECTS OF DETECTING AND PURIFYING INFLUENZA NEURAMINIDASE, which was filed on 11 Aug. 2021, and is hereby incorporated by reference in its entirety. Briefly, oseltamivir-phosphate (TAMIFLU®) is a competitive inhibitor of NA enzymatic activity. In this application, the terms oseltamivir-phosphate and TAMIFLU® are used interchangeably. The oseltamivir substrate is specific to the enzymatic site of the tetrameric NA head. In contrast, monomeric NA ectodomain variants that are enzymatically inactive, do not bind oseltamivir-phosphate. Thus, the level of recombinant NA binding in the TAMIFLU®-binding assay is proportional to the concentration of recombinant NA that are assembled as tetramers and can be measured in μg/mL. For example, the soluble tetrameric NA, as described herein, can have a TAMIFLU®-binding value of at least 5 μg/mL, such as at least 20 μg/mL, at least 50 μg/mL, or at least 100 μg/mL. The soluble tetrameric NA, as described herein, can have a TAMIFLU®-binding value of 40-1000 μg/mL, 50-800 μg/mL, 100-800 μg/mL, 100-500 μg/mL, or 100-250 μg/mL. Alternatively, the level of recombinant NA binding in the TAMIFLU®-binding assay is proportional to the concentration of recombinant NA that are assembled as tetramers and can be measured in units relative to the binding of a positive control, tetrameric NA molecule, such as a modified influenza N2 comprising a heterologous tetramerization domain, including, for example, a positive control used in this application, such as a modified A/Singapore/INFIMH160019/2016 NA comprising a full-length, tetrabrachion tetramerization domain. For example, the soluble tetrameric NA, as described herein, can have a TAMIFLU®-binding value of about 0.5 to 10 units relative to the TAMIFLU® binding of a modified A/Singapore/INFIMH160019/2016 NA comprising a full-length, tetrabrachion tetramerization domain (SEQ ID NO: 116), such as about 2 to 10 or about 5 to 10 relative units.

For purposes of determining the TAMIFLU® binding activity of a soluble tetrameric NA of the present disclosure, a TAMIFLU® binding assay should be performed using the following conditions: capture an oseltamivir-phosphate biotin conjugate (5-10 μg/ml in 1×KB buffer (0.1% BSA and 0.02% Tween 20 in PBS, pH 7.4)) on the surface of streptavidin-coated biosensors (e.g., a High Precision Streptavidin (SAX) Dip and Read Biosensor, Cat. No. 18-51182); dip the oseltamivir-phosphate bound biosensors into sample wells containing serial 2-fold dilutions of a sample of recombinant NA (0.16-40 μg/ml in 1×KB); and measure the binding kinetics of the recombinant NA to oseltamivir-phosphate using the Bio-Layer Interferometry (BLI) technique on an Octet instrument (ForteBio, Molecular Devices, LLC). If necessary, an equivalent assay can be used to measure TAMIFLU® binding.

### 10. N2 Strains and NA Sequences

The modified monomeric influenza virus NA disclosed herein is from a subtype 2 (N2) influenza virus, i.e., a modified monomeric N2. As discussed elsewhere, current seasonal influenza vaccines must be administered every year and require yearly updates to account for mutations in HA and NA proteins (antigenic drift) and to match rapidly-evolving and newly emerging viral strains. HA elicits virus-neutralizing antibodies and evolves more rapidly than NA. The more genetically stable NA could be used as a supplement in different seasonal vaccines thus increasing efficacy. In other words, a recombinant NA protein (or protein delivered by nucleic acid vaccine) may not require seasonal updates as frequently as HA. The strategy disclosed in this application for generating truncated, recombinant NA that forms tetrameric NA when expressed in cells can be used to produce modified N2 from such newly emerging N2 influenza strains, including future N2 standard of care (SOC) strains, or optionally historical SOC or non-SOC strains.

The strategy disclosed in this application for generating truncated, recombinant NA that forms tetrameric NA when expressed in cells can be used to produce modified N2 from engineered N2 proteins, such as engineered N2 proteins obtained using molecular modeling methods, as disclosed, for example, in U.S. Published Application No. 2018/0298063, U.S. Published Application No. 2019/0161519, and U.S. Published Application No. 2021/0046176, which are hereby incorporated by reference in their entirety.

The strategy disclosed in this application for generating truncated, recombinant NA that forms tetrameric NA when expressed in cells can be also used to produce modified N2 from existing wild type N2 influenza strains, including standard of care (SOC) N2 strains or non-SOC N2 strains. In some embodiments, the N2 is from a Type A influenza virus strain. In some embodiments, the N2 is from a A/(H3N2) strain. In some embodiments, the Type A influenza virus strain is one of the following strains: A/Perth/16/2009, A/Kansas/14/2017, A/Belgium/4217/2015, A/Singapore/INFIMH-16-0019/2016, A/Switzerland/8060/2017, A/Nevada/32/2013, A/Yamagata/62/1993, A/Nigeria/120/2014, A/Bangkok/i/1979, A/Albany/42/1975, A/Washington/60/2014, A/HongKong/CUHK13510/2001, A/Marrakech/79/2014, A/HongKong/1774/99, A/Illinois/34/2012, A/Kannur/MCVR5404/2010, or A/Peru/3216/2016. The SEQ ID NO: of the NA, amino acids corresponding to the expected head region, GISAID Ref. No. and Isolate ID for each of these strains are provided in the table below.

| | SEQ ID | Head | | |
|---|---|---|---|---|
| Strain | NO: | Region | GISAID | Isolate No. |
| A/Perth/16/2009 | 65 | 83-469 | EPI1211335 | EP1_ISL_60761 |
| A/Kansas/14/2017 | 45 | 83-469 | EPI1318831 | EPI_ISL_331213 |
| A/Belgium/4217/2015 | 5 | 83-469 | N/A | N/A |
| A/Singapore/INFIMH-16- | 72 | 83-469 | EPI1047603 | EPI_ISL_275709 |
| 0019/2016 | | | | |
| A/Switzerland/8060/2017 | 111 | 83-469 | EPI1313525 | EPI_ISL_330357 |
| A/Nevada/32/2013 | 108 | 83-469 | EPI503906 | EPI_ISL_154817 |
| A/Yamagata/62/1993 | 114 | 83-469 | EPI362717 | EPI_ISL_111084 |
| A/Nigeria/120/2014 | 109 | 83-469 | EPI626315 | EPI_ISL_192158 |
| A/Bangkok/1/1979 | 102 | 83-469 | EPI367160 | EPI_ISL_115698 |
| A/Albany/42/1975 | 115 | 83-469 | EPI59631 | EPI_ISL_6977 |
| A/Washington/60/2014 | 113 | 83-469 | EPI932649 | EPI_ISL_251082 |
| A/HongKong/CUHK13510/2001 | 103 | 83-469 | EPI161281 | EPI_ISL_14260 |
| A/Marrakech/79/2014 | 106 | 83-469 | EPI563208 | EPI_ISL_172394 |
| A/HongKong/1774/99 | 28 | 83-469 | EPI11604 | EPI_ISL_2466 |
| A/Illinois/34/2012 | 104 | 83-469 | EPI842693 | EPI_ISL_235247 |
| A/Kannur/MCVR5404/2010 | 105 | 83-469 | EPI839185 | EPI_ISL_234690 |
| A/Peru/3216/2016 | 110 | 83-469 | EPI861996 | EPI_ISL_238656 |

In some embodiments, the Type A influenza virus strain is one of the following strains: A/Ohio/13/2017, A/Nanjing/1663/2010, A/Fukuoka/DS7282/2017, A/Ohio/62/2012, A/Minnesota/111/2010, A/Utah/11/2011, A/Hokkaido/10H079/2011, A/Ishikawa/DS7157/2016, A/Gambia/G0071436/2012, A/Kagawa/DS722/2016, A/South_Australia/85/2018, A/Victoria/361/2011, A/Tokyo/DS7334/2017, A/Hochiminh/4596/2010, A/Kagawa/DS7144/2016, A/Peru/4617/2017, A/Switzerland/9715293/2013, A/Tokyo/UTSK1/2007, A/WestemAustralia/13/2001, A/Tasmania/1018/2015, A/Sweden/3/2017, A/Kansas/13/2009, A/Tokyo/DS763/2016, A/Newcastle/67/2016, A/SouthAustralia/34/2019, A/Stockholm/32/2014, A/Stockholm/14/2012, A/Stockholm/15/2014, A/Guangxigangbei/190/2019, A/Xinjiangtianshan/1411/2012, A/NewYork/654/1994, A/Netherlands/620/1989, A/NewYork/758/1993, A/Catalonia/9503S/2017, A/Paris/2379/2014, or A/Heilongjiangxiangyang/1134/2011. The SEQ ID NO: of the NA, amino acids corresponding to the expected head region, GISAID Ref. No. and Isolate ID for each of these strains are provided in the table below.

| | SEQ ID | Head | | |
|---|---|---|---|---|
| Strain | NO: | Region | GISAID | Isolate No. |
| A/Ohio/13/2017 | 61 | 83-469 | EPI1056652 | EPI_ISL_277234 |
| A/Nanjing/1663/2010 | 55 | 83-469 | EPI297539 | EPI_ISL_84852 |
| A/Fukuoka/DS7282/2017 | 13 | 83-469 | EPI1303010 | EPI_ISL_327968 |
| A/Ohio/62/2012 | 62 | 83-469 | EPI394905 | EPI_ISL_128388 |
| A/Minnesota/11/2010 | 50 | 83-469 | EPI465399 | EPI_ISL_145077 |
| A/Utah/11/2011 | 96 | 83-469 | EPI316206 | EPI_ISL_89828 |
| A/Hokkaido/10H079/2011 | 25 | 83-469 | EPI340631 | EPI_ISL_98459 |
| A/Ishikawa/DS7157/2016 | 37 | 83-469 | EPI1302992 | EPI_ISL_327951 |
| A/Gambia/G0071436/2012 | 14 | 83-469 | EPI536339 | EPI_ISL_164225 |
| A/Kagawa/DS722/2016 | 40 | 83-469 | EPI1303003 | EPI_ISL_327961 |
| A/South_Australia/85/2018 | 74 | 83-469 | EPI1370000 | EPI_ISL_341299 |
| A/Victoria/361/2011 | 97 | 83-469 | EPI551805 | EPI_ISL_168948 |
| A/Tokyo/DS7334/2017 | 92 | 83-468 | EPI1303022 | EPI_ISL_327980 |
| A/Hochiminh/4596/2010 | 24 | 83-469 | EPI535015 | EPI_ISL_163974 |
| A/Kagawa/DS7144/2016 | 43 | 83-469 | EPI1302984 | EPI_ISL_327944 |
| A/Peru/4617/2017 | 67 | 83-469 | EPI1174550 | EPI_ISL_298374 |
| A/Switzerland/9715293/2013 | 79 | 83-469 | EPI543762 | EPI_ISL_166859 |
| A/Tokyo/UTSK1/2007 | 94 | 83-469 | EPI241271 | EPI_ISL_69392 |
| A/WesternAustralia/13/2001 | 99 | 83-469 | EPI44542 | EPI_ISL_6173 |
| A/Tasmania/1018/2015 | 84 | 83-470 | EPI675974 | EPI_ISL_202620 |
| A/Sweden/3/2017 | 78 | 83-469 | EPI945994 | EPI_ISL_253859 |
| A/Kansas/13/2009 | 44 | 83-469 | EPI465396 | EPI_ISL_145076 |
| A/Tokyo/DS763/2016 | 90 | 83-468 | EPI1302928 | EPI_ISL_327894 |
| A/Newcastle/67/2016 | 60 | 83-470 | EPI831629 | EPI_ISL_233278 |
| A/SouthAustralia/34/2019 | 73 | 83-469 | EPI1387330 | EPI_ISL_346283 |
| A/Stockholm/32/2014 | 77 | 83-469 | EPI558926 | EPI_ISL_170831 |
| A/Stockholm/14/2012 | 75 | 83-469 | EPI369152 | EPI_ISL_117719 |
| A/Stockholm/15/2014 | 76 | 83-469 | EPI531449 | EPI_ISL_162466 |
| A/Guangxigangbei/190/2019 | 17 | 83-469 | EPI1503951 | EPI_ISL_367161 |
| A/Xinjiangtianshan/1411/2012 | 101 | 83-469 | EPI408590 | EPI_ISL_133063 |
| A/NewYork/654/1994 | 58 | 83-469 | EPI34717 | EPI_ISL_5396 |
| A/Netherlands/620/1989 | 56 | 83-469 | EPI297834 | EPI_ISL_84905 |
| A/NewYork/758/1993 | 59 | 83-469 | EPI36237 | EPI_ISL_5475 |
| A/Catalonia/9503S/2017 | 9 | 83-469 | EPI1015697 | EPI_ISL_267962 |
| A/Paris/2379/2014 | 64 | 83-469 | EPI574238 | EPI_ISL_174888 |
| A/Heilongjiangxiangyang/1134/2011 | 21 | 83-469 | EPI330528 | EPI_ISL_94612 |

The Type A influenza strain may also be one of the following: A/TEXAS/50/2012

(SEQ ID NO: 87)
A/TEXAS/50/2012,
(SEQ ID NO: 69)
A/PORTOALEGRE/LACENRS2376/2014,
(SEQ ID NO: 49)
A/MEMPHIS/18/1978,
(SEQ ID NO: 5)
A/BELGIUM/4217/2015,
(SEQ ID NO: 81)
A/TAIWAN/1/1969,
(SEQ ID NO: 71)
A/SIENA/3/1991,
(SEQ ID NO: 7)
A/BRISBANE/273/2016,
(SEQ ID NO: 63)
A/ONTARIO/RV3236/2016,
(SEQ ID NO: 85)
A/TEHRAN/996/2012,
(SEQ ID NO: 3)
A/ANKARA/2396/2015,
(SEQ ID NO: 45)
A/KANSAS/14/2017,
(SEQ ID NO: 88)
A/TEXAS/71/2017,
(SEQ ID NO: 30)
A/HONGKONG/4801/2014,
(SEQ ID NO: 29)
A/HONGKONG/3089/2017,
(SEQ ID NO: 12)
A/FUKUOKA/DS729/2016,
(SEQ ID NO: 89)
A/TOKUSHIMA/DS5288/2015,
(SEQ ID NO: 22)
A/HELSINKI/823/2013,
(SEQ ID NO: 1)
A/ALASKA/251/2015,
(SEQ ID NO: 20)
A/HATAY/4990/2016,
(SEQ ID NO: 19)
A/HANOI/ELI15597/2015,
(SEQ ID NO: 51)
A/MISSISSIPPI/1/1985,
(SEQ ID NO: 36)
A/INDIANA/18/2017,
(SEQ ID NO: 68)
A/POLAND/19B/2017,
(SEQ ID NO: 86)
A/TENNESSEE/18/2017,
(SEQ ID NO: 28)
A/HONGKONG/1774/1999,
(SEQ ID NO: 10)
A/DAKAR/14/2014,
(SEQ ID NO: 70)
A/SAUDIARABIA/21/1999,
(SEQ ID NO: 15)
A/GIFU/DS7388/2017,
(SEQ ID NO: 57)
A/NEWYORK/581/1997,
(SEQ ID NO: 23)
A/HELSINKI/941/2013,
(SEQ ID NO: 39)
A/ISHIKAWA/DS7294/2017,
(SEQ ID NO: 16)
A/GUANGDONGDUANZHOU/1227/2017,
(SEQ ID NO: 4)
A/AUCKLAND/5/1996,
(SEQ ID NO: 38)
A/ISHIKAWA/DS7215/2016,
(SEQ ID NO: 98)
A/WESTVIRGINIA/17/2012,
(SEQ ID NO: 72)
A/SINGAPORE/INFIMH160019/2016,
(SEQ ID NO: 42)
A/KAGAWA/DS7115/2016,
(SEQ ID NO: 6)
A/BILTHOVEN/21801/1971,
(SEQ ID NO: 11)
A/FUKUOKA/DS72/2016,
(SEQ ID NO: 52)
A/MORAMANGA/1907/2017,
(SEQ ID NO: 33)
A/HUNAN/01/2014,
(SEQ ID NO: 100)
A/WISCONSIN/16/2015,
(SEQ ID NO: 8)
A/CANBERRA/13/2015,
(SEQ ID NO: 27)
A/HONGKONG/107/1971,
(SEQ ID NO: 48)
A/LYON/1242/2000,
(SEQ ID NO: 53)
A/NAGANO/2153/2017,
(SEQ ID NO: 54)
A/NANJING/49/1977,
(SEQ ID NO: 2)
A/ALBANY/6/1970,
(SEQ ID NO: 41)
A/KAGAWA/DS769/2016,
(SEQ ID NO: 83)
A/TAIWAN/4183/2004,
(SEQ ID NO: 47)
A/KOREA/KUMCGR570/2011,
(SEQ ID NO: 80)
A/SYDNEY/24/2015,
(SEQ ID NO: 46)
A/KOREA/KUMCGR99/2011,
(SEQ ID NO: 91)
A/TOKYO/DS7277/2017,
(SEQ ID NO: 31)
A/HONGKONG/CUHK18194/1998,
(SEQ ID NO: 18)
A/GUNMA/DS7107/2016,
(SEQ ID NO: 26)
A/HONGKONG/8/1968,
(SEQ ID NO: 93)
A/TOKYO/DS7448/2017,
(SEQ ID NO: 65)
A/PERTH/61/2015,
(SEQ ID NO: 95)
A/TW/875/2004,
(SEQ ID NO: 82)
A/TAIWAN/2332/2001,
(SEQ ID NO: 34)
A/INDIA/C058671/2005.

The modified monomeric N2 disclosed herein can comprise a portion of a wild type N2 (e.g., the head region or part of the C-terminal region of the stalk region of the wild type N2). It is also possible to introduce one or more mutations into the head region and/or the C-terminal region of the stalk region of the wild type N2, as long as the modified monomeric N2 retains the ability to form a tetrameric NA when expressed in cells. In certain embodiments, the head region and/or the C-terminal region of the stalk region of the modified monomeric N2 contains 1, 2, 3, 4, or 5 mutations relative to the head region and/or the C-terminal region of the stalk region of the wild type N2. In certain embodiments, the modified monomeric N2 comprises at least 90%, 91%, 92%, 93%, 94%, 95%, 96%, 97% 98%, or 99% identity with the head region of a wild type N2. In certain embodiments, the modified monomeric N2 comprises at least 90%, 91%, 92%, 93%, 94%, 95%, 96%, 97% 98%, or 99% identity with the head region of any of the amino acid sequences of SEQ ID NO: 1-115.

### C. Compositions

In certain aspects, the present disclosure provides vaccine compositions comprising a tetrameric neuraminidase, comprising four copies of a modified monomeric N2, as described herein, or an artificial nucleic acid molecule encoding the modified monomeric N2, as described herein. Nucleic acid vaccines based on plasmid DNA, vectors, or RNA (e.g., mRNA) are known in the art and have been used effectively against infectious diseases, such as viral infections. Vectors include, but are not limited to, in vivo recombinant expression vectors, such as a polynucleotide vector or a plasmid (EP-A2-1001025; Chaudhuri P, Res. Vet. Sci. 2001, 70: 255-6), virus vectors, including, but not limited to, adenovirus vectors, alphavirus vectors, flavivirus vectors, poxvirus vectors such as fowlpox (U.S. Pat. Nos. 5,174,993; 5,505,941; and 5,766,599) or canarypox vectors (U.S. Pat. No. 5,756,103). RNA (e.g., mRNA) vaccines can be formulated with a lipid nanoparticle. The artificial nucleic acid encoding the modified monomeric N2, as described herein, can be stabilized by one or more chemical modifications to the nucleic acid molecule, including one or more backbone, sugar, and/or base modifications, as known in the art, including, for example, as disclosed in PCT Publication No. WO2017/140905, which is hereby incorporated by reference in its entirety.

In certain embodiments, the present disclosure provides vaccine compositions comprising mRNA molecules encoding a modified monomeric N2 as described herein. In an embodiment, the mRNA molecules are encapsulated in lipid nanoparticles (LNPs). LNPs for delivering mRNA vaccines are known in the art, including, for example, as disclosed in US Patent Application Nos. 63/110,965 and 63/212,523, which are hereby incorporated by reference in their entireties.

The vaccine composition can also further comprise an adjuvant.

As used herein, the term “adjuvant” refers to a substance or vehicle that non-specifically enhances the immune response to an antigen. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, J. Immunol., 2009, 183: 6186-6197), IL-2, RANTES, GM-CSF, TNF-α, IFN-γ, G-CSF, LFA-3, CD72, B7-1, B7-2, OX-40L and 41 BBL.

In certain embodiments, the adjuvant is a squalene-based adjuvant comprising an oil-in-water adjuvant emulsion comprising at least: squalene, an aqueous solvent, a polyoxyethylene alkyl ether hydrophilic nonionic surfactant, and a hydrophobic nonionic surfactant. In certain embodiments, the emulsion is thermoreversible, optionally wherein 90% of the population by volume of the oil drops has a size less than 200 nm.

In certain embodiments, the polyoxyethylene alkyl ether is of formula CH₃—(CH₂)ₓ—(O—CH₂—CH₂)ₙ—OH, in which n is an integer from 10 to 60, and x is an integer from 11 to 17. In certain embodiments, the polyoxyethylene alkyl ether surfactant is polyoxyethylene(12) cetostearyl ether.

In certain embodiments, 90% of the population by volume of the oil drops has a size less than 160 nm. In certain embodiments, 90% of the population by volume of the oil drops has a size less than 150 nm. In certain embodiments, 50% of the population by volume of the oil drops has a size less than 100 nm. In certain embodiments, 50% of the population by volume of the oil drops has a size less than 90 nm.

In certain embodiments, the adjuvant further comprises at least one alditol, including, but not limited to, glycerol, erythritol, xylitol, sorbitol and mannitol.

In certain embodiments the hydrophilic/lipophilic balance (HLB) of the hydrophilic nonionic surfactant is greater than or equal to 10. In certain embodiments, the HLB of the hydrophobic nonionic surfactant is less than 9. In certain embodiments, the HLB of the hydrophilic nonionic surfactant is greater than or equal to 10 and the HLB of the hydrophobic nonionic surfactant is less than 9.

In certain embodiments, the hydrophobic nonionic surfactant is a sorbitan ester, such as sorbitan monooleate, or a mannide ester surfactant. In certain embodiments, the amount of squalene is between 5 and 45%. In certain embodiments, the amount of polyoxyethylene alkyl ether surfactant is between 0.9 and 9%. In certain embodiments, the amount of hydrophobic nonionic surfactant is between 0.7 and 7%. In certain embodiments, the adjuvant comprises: i) 32.5% of squalene, ii) 6.18% of polyoxyethylene(12) cetostearyl ether, iii) 4.82% of sorbitan monooleate, and iv) 6% of mannitol.

In certain embodiments, the adjuvant further comprises an alkylpolyglycoside and/or a cryoprotective agent, such as a sugar, in particular dodecylmaltoside and/or sucrose.

In certain embodiments, the adjuvant comprises AF03, as described in Klucker et al., J. Pharm. Sci. 2012, 101(12):4490-500, which is hereby incorporated by reference in its entirety.

In addition to the tetrameric neuraminidase or the artificial nucleic acid molecule encoding the modified monomeric N2 and optional adjuvant, the vaccine composition may also further comprise one or more pharmaceutically acceptable excipients. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, vaccine compositions to be administered can contain minor amounts of non-toxic auxiliary substances, such as wetting or emulsifying agents, pharmaceutically acceptable salts to adjust the osmotic pressure, preservatives, stabilizers, buffers, sugars, amino acids, and pH buffering agents and the like, for example sodium acetate or sorbitan monolaurate.

Typically, the vaccine composition is a sterile, liquid solution formulated for parenteral administration, such as intravenous, subcutaneous, intraperitoneal, intradermal, or intramuscular. The vaccine composition may also be formulated for intranasal or inhalation administration. The vaccine composition can also be formulated for any other intended route of administration.

In some embodiments, a vaccine composition is formulated for intradermal injection, intranasal administration or intramuscular injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19^{th} ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the vaccine composition is administered using a device that delivers a metered dosage of the vaccine composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO1997/37705, and WO1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The vaccine composition may further comprise an influenza virus hemagglutinin protein. In certain embodiments, the influenza virus hemagglutinin and the modified influenza virus subtype 2 neuraminidase are from different influenza virus strains. In certain embodiments, the influenza virus hemagglutinin and the modified influenza virus subtype 2 neuraminidase are from different Type A influenza virus strains. In certain embodiments, the influenza virus hemagglutinin and the modified influenza virus subtype 2 neuraminidase are from different A/(H3N2) virus strains.

### D. Nucleic Acids, Cloning, and Expression Systems

The present disclosure further provides artificial nucleic acid molecules encoding the disclosed modified monomeric N2. The nucleic acids may comprise DNA or RNA and may be wholly or partially synthetic or recombinant. Reference to a nucleotide sequence as set out herein encompasses a DNA molecule with the specified sequence and encompasses an RNA molecule with the specified sequence in which U is substituted for T, or a derivative thereof, such as pseudouridine, unless context requires otherwise. Other nucleotide derivatives or modified nucleotides can be incorporated into the artificial nucleic acid molecules encoding the disclosed modified monomeric N2.

The present disclosure also provides constructs in the form of a vector (e.g., plasmids, phagemids, cosmids, transcription or expression cassettes, artificial chromosomes, etc.) comprising an artificial nucleic acid molecule encoding a modified monomeric N2. The disclosure further provides a host cell which comprises one or more constructs as above.

Also provided are methods of making the modified monomeric N2 encoded by these artificial nucleic acid molecules. The modified N2 polypeptides may be produced using recombinant techniques. The production and expression of recombinant proteins is well known in the art and can be carried out using conventional procedures, such as those disclosed in Sambrook et al., Molecular Cloning: A Laboratory Manual (4th Ed. 2012), Cold Spring Harbor Press. For example, expression of the modified N2 polypeptide may be achieved by culturing under appropriate conditions host cells containing the artificial nucleic acid molecule encoding the modified monomeric N2 polypeptide. Thus, a method for producing tetrameric NA may comprise culturing host cells in a cell culture medium, wherein the host cells contain an artificial nucleic acid encoding the modified monomeric N2, and expressing the modified N2 in the host cells, wherein modified N2 is secreted from the host cells as soluble, tetrameric NA. Following production by expression, the tetrameric NA may be isolated and/or purified using any suitable technique, then used as appropriate.

Systems for cloning and expression of a polypeptide in a variety of different host cells are well known in the art. Any protein expression system (e.g., stable or transient) compatible with the constructs disclosed in this application may be used to produce the modified N2 described herein.

Suitable vectors can be chosen or constructed, so that they contain appropriate regulatory sequences, including promoter sequences, terminator sequences, polyadenylation sequences, enhancer sequences, marker genes and other sequences as appropriate.

A further aspect of the disclosure provides a host cell comprising an artificial nucleic acid molecule as disclosed herein. A still further aspect provides a method comprising introducing such artificial nucleic acid molecules into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. These techniques are well known in the art. (See, e.g., “Current Protocols in Molecular Biology,” Ausubel et al. eds., John Wiley & Sons, 2010). DNA introduction may be followed by a selection method (e.g., antibiotic resistance) to select cells that contain the vector.

The host cell may be a plant cell, a yeast cell, or an animal cell. Animal cells encompass invertebrate (e.g., insect cells), non-mammalian vertebrate (e.g., avian, reptile and amphibian) and mammalian cells. In one embodiment, the host cell is a mammalian cell. Examples of mammalian cells include, but are not limited to COS-7 cells, HEK293 cells; baby hamster kidney (BHK) cells; Chinese hamster ovary (CHO) cells; mouse sertoli cells; African green monkey kidney cells (VERO-76); human cervical carcinoma cells (e.g., HeLa); canine kidney cells (e.g., MDCK), and the like. In one embodiment, the host cells are CHO cells.

### D. Methods of Production

The truncated NA stalk design disclosed herein allows for large-scale production and purification of soluble, tetrameric NA. As described above, a vector comprising an artificial nucleic acid molecule encoding the disclosed modified monomeric N2 can be inserted into a suitable host cell to produce soluble, tetrameric NA.

In some embodiments, the method of producing the soluble, tetrameric NA uses large-scale production conditions. As used herein, “large-scale production conditions” refer to cultivating host cells in a culture vessel, typically a bioreactor, with a working volume of between 10 and 10,000 liters, between 25 and 5000 liters, between 25 and 2000 liters, between 50 liters and 1000 liters, between 50 and 500 liters, between 50 and 250 liters, between 50 and 200 liters, between 100 and 200 liters, between 100 liters and 5000 liters, between 500 liters and 8000 liters, between 1500 liters and 6500 liters, about 1500-1600 liters, about 3000-3200 liters, about 6000-6400 liters, greater than or equal to 25 liters, such as greater than or equal to 100 liters, such as at least 100 liters and less than or equal to 10,000 liters, such as at least 100 liters and less than or equal to 8000 liters, such as at least 100 liters and less than or equal to 4000 liters or such as at least 100 liters and less than or equal to 2000 liters.

As described herein, the soluble tetrameric NA described herein can be purified from cell culture supernatants using chromatography techniques, including, for example, ion exchange chromatography, affinity chromatography, and size exclusion chromatography. For example, if the modified N2 contains a charged tag, such as a histidine tag, the soluble NA can be purified using ion exchange chromatography. In some embodiments, soluble, tetrameric NA, as described herein, is purified using size exclusion chromatography (SEC). In addition, SEC-Multi-Angle Light Scattering (SEC-MALS) can be used, for example, to determine both molecular weight and purity of the soluble NA molecule.

In some N2 influenza strains, when the modified monomeric N2 is expressed in host cells and the host cell supernatant host cell supernatant contains a mixture of modified NA comprising a monomeric form of the modified NA, a tetrameric form of the modified NA, and/or other oligomeric forms of the modified NA, such as dimeric NA, trimeric NA, and/or higher order oligomeric NA (e.g., an aggregation or other multimer of modified monomeric, dimeric, trimeric or tetrameric NA). In some N2 influenza strains, wherein the host cell supernatant is applied to a chromatography column, the soluble NA may elute in two main peaks or pools, whereas for other N2 influenza strains, the soluble NA may elute as a single peak or pool. For some N2 influenza strains, when the modified monomeric N2 is expressed in cells and the cell supernatant is applied to a chromatography column, the soluble NA may elute in more than two main peaks or pools. When the soluble NA elutes in two peaks or pools, the first peak or pool contains predominately monomeric NA, having a molecular weight of about 60 kD, while the second peak contains predominantly tetrameric NA having a molecular weight of about 210 kD. When the soluble NA elutes as a single peak or pool, the pool typically contains a mixture of monomeric and tetrameric NA. The purity of the tetrameric NA in the cell supernatant can vary between different N2 strains, with certain strains, like PERT09 and BELG17, which elute with a single peak or pool, containing about 70-100% and 20-25% tetrameric NA, respectively, in the single peak or pool. In some embodiments, the soluble tetrameric NA represents at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the modified N2 in the host cell supernatant, as measured using SEC. In some embodiments, the soluble tetrameric NA is present in the host cell supernatant in an amount of about 50-150 mg/L, including for example, about 75-140 mg/L, about 80-135 mg/L or about 80 mg/L to 120 mg/L. If desired, soluble, tetrameric NA obtained from the host cell supernatant can be further purified.

In some embodiments, soluble, tetrameric NA, as described herein, is purified using affinity chromatography. For example, soluble tetrameric NA can be purified using a new TAMIFLU® binding assay, as described in the examples. The TAMIFLU® purification method is also described in U.S. Provisional Patent Application No. 63/231,795, entitled METHODS AND RELATED ASPECTS OF DETECTING AND PURIFYING INFLUENZA NEURAMINIDASE, which was filed on 11 Aug. 2021, and is hereby incorporated by reference in its entirety. As it was found that recombinant monomeric NA does not bind to TAMIFLU®, the TAMIFLU® purification method provides a convenient way to purify soluble tetrameric NA in mixture comprising soluble tetrameric and monomeric NA. In some embodiments, a TAMIFLU® binding conjugate is used to purify soluble, tetrameric NA from cell culture supernatants. In some embodiments, the cell culture supernatants are subjected to a first purification method, such as ion exchange chromatography or SEC, to obtain a partially purified peak or pool containing a mixture of monomeric and tetrameric NA, followed by a second purification method, wherein the second purification method is a TAMIFLU®-based purification method comprising a step of incubating the mixture comprising monomeric NA and tetrameric NA with a TAMIFLU® conjugate and isolating the tetrameric NA bound to the TAMIFLU® conjugate to purify the tetrameric NA.

In some embodiments, following purification, a purified composition is obtained, the composition comprising at least 90% tetrameric NA. In some embodiments, the purified sample comprises at least 91%, 92%, 93%, 94%, 95%, 96%, 97%, 98%, or 99% tetrameric NA. In some embodiments, following purification, a purified composition is obtained, the composition comprising tetrameric NA at a concentration of at least 0.01, 0.2, 0.5, 1, 2, 3, 4, or 5 mg/mL, such as 0.01 mg/mL to about 25 mg/mL, 0.2 mg/mL to about 25 mg/mL, such as 0.5 mg/mL to 10 mg/mL, 2 mg/mL to 10 mg/mL, 0.5 mg/mL to 20 mg/mL, 2 mg/mL to 20 mg/mL, 0.5 mg/mL to 15 mg/mL, or 2 mg/mL to 15 mg/mL.

### E. Methods of Use

The present disclosure provides methods of administering the vaccine compositions described herein to a subject. The methods may be used to vaccinate a subject against an influenza virus. In some embodiments, the vaccination method comprises administering to a subject in need thereof a vaccine composition comprising an artificial nucleic acid molecule encoding a modified monomeric N2, as described herein, or a tetrameric NA, comprising four copies of a modified monomeric N2, as described herein, and an optional adjuvant in an amount effective to vaccinate the subject against influenza virus. Likewise, the present disclosure provides a vaccine composition comprising an artificial nucleic acid molecule encoding a modified monomeric N2, as described herein, or a tetrameric NA, comprising four copies of a modified monomeric N2, as described herein, and an optional adjuvant, for use in vaccinating a subject against an influenza virus.

The present disclosure also provides methods of immunizing a subject against influenza virus, comprising administering to the subject an immunologically effective amount of a vaccine composition comprising an artificial nucleic acid molecule encoding a modified monomeric N2, as described herein, or a tetrameric NA, comprising four copies of a modified monomeric N2, as described herein, and an optional adjuvant. Likewise, the present disclosure provides a vaccine composition comprising an artificial nucleic acid molecule encoding a modified monomeric N2, as described herein, or a tetrameric NA, comprising four copies of a modified monomeric N2, as described herein, and an optional adjuvant, for use in immunizing a subject against an influenza virus.

In some embodiments, the method or use prevents influenza virus infection or disease in the subject. In some embodiments, the method or use raises a protective immune response in the subject. In some embodiments, the protective immune response is an antibody response.

The methods of immunizing provided herein can elicit a broadly neutralizing immune response against one or more influenza viruses. Accordingly, in various embodiments, the composition described herein can offer broad cross-protection against different types of influenza viruses. In some embodiments, the composition offers cross-protection against avian, swine, seasonal, and/or pandemic influenza viruses. In some embodiments, the methods of immunizing are capable of eliciting an improved immune response against one or more seasonal influenza strains (e.g., a standard of care strain). For example, the improved immune response may be an improved humoral immune response. In some embodiments, the methods of immunizing are capable of eliciting an improved immune response against one or more pandemic influenza strains. In some embodiments, the methods of immunizing are capable of eliciting an improved immune response against one or more swine influenza strains. In some embodiments, the methods of immunizing are capable of eliciting an improved immune response against one or more avian influenza strains.

Also provided are methods of preventing influenza virus disease in a subject, comprising administering to the subject a vaccine composition comprising an artificial nucleic acid molecule encoding a modified monomeric N2, as described herein, or a tetrameric NA, comprising four copies of a modified monomeric N2, as described herein, and an optional adjuvant in an amount effective to prevent influenza virus disease in the subject. Likewise, the present disclosure provides a vaccine composition comprising an artificial nucleic acid molecule encoding a modified monomeric N2, as described herein, or a tetrameric NA, comprising four copies of a modified monomeric N2, as described herein, and an optional adjuvant, for use in preventing influenza virus disease in a subject.

Also provided are methods of inducing an immune response against an influenza virus NA in a subject, comprising administering to the subject a vaccine composition comprising an artificial nucleic acid molecule encoding a modified monomeric N2, as described herein, or a tetrameric NA, comprising four copies of a modified monomeric N2, as described herein, and an optional adjuvant. Likewise, the present disclosure provides a vaccine composition comprising an artificial nucleic acid molecule encoding a modified monomeric N2, as described herein, or a tetrameric NA, comprising four copies of a modified monomeric N2, as described herein, and an optional adjuvant, for use in inducing an immune response against an influenza virus NA in a subject.

Vaccine compositions comprising an artificial nucleic acid molecule encoding a modified monomeric N2, as described herein, or a tetrameric NA, comprising four copies of a modified monomeric N2, as described herein and an optional adjuvant may be administered prior to or after development of one or more symptoms of an influenza infection. That is, in some embodiments, the vaccine compositions described herein may be administered prophylactically to prevent influenza infection or ameliorate the symptoms of a potential influenza infection. In some embodiments, a subject is at risk of influenza virus infection if the subject will be in contact with other individuals or livestock (e.g., swine) known or suspected to have been infected with pandemic influenza virus and/or if the subject will be present in a location in which influenza infection is known or thought to be prevalent or endemic. In some embodiments, the vaccine compositions are administered to a subject suffering from an influenza infection, or the subject is displaying one or more symptoms commonly associated with influenza infection. In some embodiments, the subject is known or believed to have been exposed to an influenza virus. In some embodiments, a subject is at risk or susceptible to an influenza infection if the subject is known or believed to have been exposed to the influenza virus. In some embodiments, a subject is known or believed to have been exposed to the influenza virus if the subject has been in contact with other individuals or livestock (e.g., swine) known or suspected to have been infected with pandemic influenza virus and/or if the subject is or has been present in a location in which influenza infection is known or thought to be prevalent or endemic. The vaccine compositions disclosed herein may be used to treat or prevent disease caused by either or both a seasonal or a pandemic influenza strain.

Vaccine compositions in accordance with the disclosure may be administered in any amount or dose appropriate to achieve a desired outcome. In some embodiments, the desired outcome is induction of a lasting adaptive immune response against a broad spectrum of influenza strains, including both seasonal and pandemic strains. In some embodiments, the desired outcome is reduction in intensity, severity, and/or frequency, and/or delay of onset of one or more symptoms of influenza infection. The dose required may vary from subject to subject depending on the species, age, weight and general condition of the subject, the severity of the infection being treated, the particular composition being used and its mode of administration.

In various embodiments, the vaccine compositions described herein are administered to subjects, wherein the subjects can be any member of the animal kingdom. In some embodiments, the subject is a non-human animal. In some embodiments, the non-human subject is an avian (e.g., a chicken or a bird), a reptile, an amphibian, a fish, an insect, and/or a worm. In some embodiments, the non-human subject is a mammal (e.g., a rodent, a mouse, a rat, a rabbit, a monkey, a dog, a cat, a sheep, cattle, a primate, and/or a pig).

In some embodiments, the vaccine compositions described herein are administered to a human subject. In particular embodiments, a human subject is 6 months of age or older, is 6 months through 35 months of age, is 36 months through 8 years of age, or 9 years of age or older. In some embodiments, the human subject is an infant (less than 36 months). In some embodiments, the human subject is a child or adolescent (less than 18 years of age). In some embodiments, the human subject is elderly (at least 60 years of age or at least 65 years of age). In some embodiments, the human subject is a non-elderly adult (at least 18 years of age and less than 65 years of age).

In some embodiments, the methods and uses of the vaccine compositions described herein include prime-boost vaccination strategies. Prime-boost vaccination comprises administering a priming vaccine and then, after a period of time has passed, administering to the subject a boosting vaccine. The immune response is “primed” upon administration of the priming vaccine and is “boosted” upon administration of the boosting vaccine. The priming vaccine can include a vaccine composition comprising an artificial nucleic acid molecule encoding a modified monomeric N2, as described herein, or a tetrameric NA comprising four copies of a modified monomeric N2, as described herein, and an optional adjuvant. Likewise, the boosting vaccine can include a vaccine composition comprising an artificial nucleic acid molecule encoding a modified monomeric N2, as described herein, or a tetrameric neuraminidase, comprising four copies of a modified monomeric N2, as described herein, and an optional adjuvant. The priming vaccine composition can be, but need not be, the same as the boosting vaccine. Administration of the boosting vaccine is generally weeks or months after administration of the priming composition, preferably about 2-3 weeks or 4 weeks, or 8 weeks, or 16 weeks, or 20 weeks, or 24 weeks, or 28 weeks, or 32 weeks.

The vaccine composition can be administered using any suitable route of administration, including, for example, parenteral delivery, as discussed above.

Typically, the artificial nucleic acid molecule encoding a modified monomeric N2, as described herein, or a tetrameric NA comprising four copies of a modified monomeric N2, as described herein, and adjuvant are administered together as components of the same vaccine composition. However, it is not necessary for the artificial nucleic acid molecule or tetrameric NA and adjuvant to be administered as part of the same vaccine composition. That is, if desired, the artificial nucleic acid molecule or the tetrameric NA and the adjuvant can be administered to the subject sequentially.

The present disclosure will be more fully understood by reference to the following Examples.

### EXAMPLES

### Example 1. Design of NA with Truncated Stalk Region

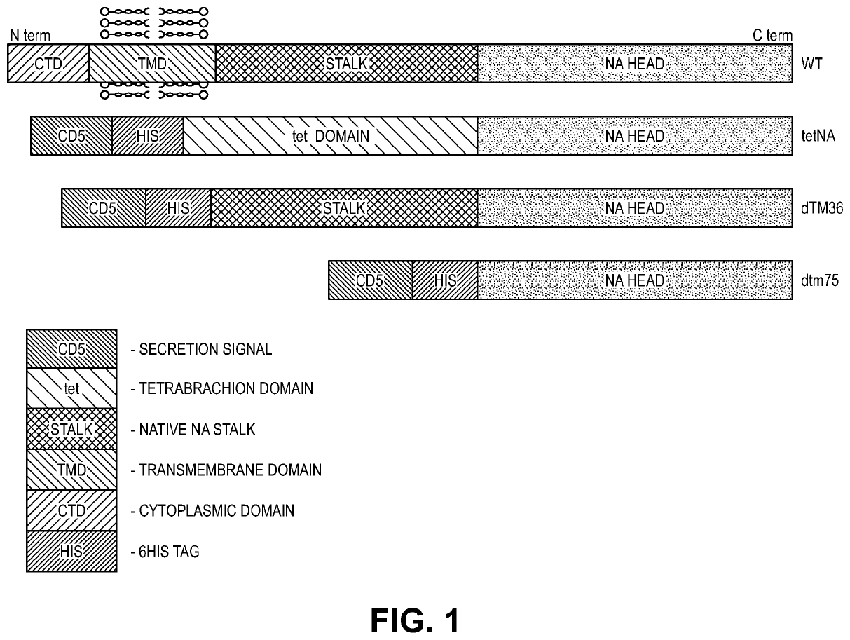

An engineered subtype 2 influenza neuraminidase (“N2”) having a truncated stalk region was designed, as shown in FIG. 1, in which amino acids 1-74 of the wild-type neuraminidase, including the cytoplasmic domain, the transmembrane region, and substantially all of stalk region are replaced by a secretion signal and an optional histidine-tag, which can be used to purify the truncated N2. This engineered N2 is called dTM75. As the truncated N2 can be purified by methods that do not involve a histidine tag, it is not necessary for the recombinant truncated stalk constructs to include this histidine tag. Another truncated N2 was designed in which the cytoplasmic domain and the transmembrane region (amino acids 1-35) were replaced by a secretion signal and a histidine-tag. This construct is referred to as dTM36 in FIG. 1 and includes the entire stalk region. Also depicted in FIG. 1 is 1) an engineered N2 in which the cytoplasmic domain, the transmembrane region, and substantially all of stalk region are replaced by a secretion signal, a histidine-tag, and a full-length, heterologous tetrabrachion tetramerization domain (“tet-NA”); and 2) a wild-type N2 (“WT”), having a short, N-terminal cytoplasmic domain, a transmembrane region, a stalk region and a head region. The N2 sequence from A/Singapore/INFIMH-16-0019/2016 (SEQ ID NO: 72) was used in initial experiments as a representative N2 sequence. The amino acid sequence of the tet-NA construct is set forth in SEQ ID NO: 116.

The dTM36, dTM75, and tet-NA constructs were expressed in CHO cells and purified to near homogeneity for further characterization. Enzymatic activity was measured by a MUNANA assay and specific activity was expressed in nmol/min/μg protein. The MUNANA assay is based on a previously described method (Potier et al. Anal. Biochem. 94, 287-296, 1979) with modifications. Briefly, 2′-(4-methylumbelliferyl)-alpha-D-N-acetylneuraminic acid (MUNANA) is used as a substrate. The neuraminidase contained in the sample cleaves the MUNANA substrate, releasing 4-Methylumbelliferone (4-MU), a fluorescent compound. The neuraminidase activity of a test sample is determined by measuring the fluorescence intensity (RFU, Relative Fluorescence Unit). In addition, a standard curve for a NA sample with known concentration can be used to determine NA concentration (μg/ml) in a test sample.

The MUNANA reaction was initiated by the addition of buffer [33.3 mM 2-(N-morpholino) ethanesulfonic acid (MES, pH 6.5), 4 mM CaCl₂), 50 mM BSA] and substrate (100 μM MUNANA). After 1 hour of incubation (37° C. with shaking), the reaction was stopped by the addition of alkaline pH solution (0.2M Na₂CO₃). The fluorescence intensity was detected on a SpectraMax M5 (Molecular Devices), using excitation and emission wavelengths of 355 and 460 nm, respectively. The enzymatic activity was calculated against a 4MU reference, and the results are expressed in μM/60 min for total NA activity and nmole/min/μg for specific NA activity.

The dTM36 variant was much less enzymatically active than tet-NA, while the dTM75 variant had similar/higher activity to tet-NA. By Size Exclusion Chromatography with Multi-Angle Light Scattering (SEC-MALS) analysis (described in Example 4), tet-NA was tetrameric, dTM36 was mostly monomeric (90%) with less than 10% tetramer, and dTM75 was at least 90% tetrameric (Table 1). Based on this result, the dTM75 design was chosen for further evaluation in other N2 strains.

TABLE 1

| | Specific NA | |
|---|---|---|
| Sample | Activity | Oligomeric status |
| Tet-NA_SG16 | 9.33 | Tetramer |
| dTM36_SG16 | 0.34 | 90% monomer |
| dTM75_SG16 | 14.57 | At least 90% tetramer |

### Example 2. High-Throughput (HT) Screening of dTM75 from 37 Different N2 Strains

Next, dTM75 constructs were synthesized using the N2 sequences from 37 naturally occurring subtype 2 influenza strains and screened for NA activity and tetramer formation. The dTM75 constructs were inserted into a plasmid, and the plasmid was transfected into ExpiCHO cells using the Expifectamine CHO transfection kit (ThermoFisher Scientific, Waltham, MA) following the manufacturer's protocol. After 4-5 days in culture, supernatant containing the soluble, truncated NA proteins was clarified, filtered and aliquoted for further characterization, including NA activity and tetramer formation.

Clarified supernatants were tested for neuraminidase activity levels using the NA-Star® assay (ThermoFisher Scientific, Waltham, MA) following the manufacturer's protocol. Samples were collected in 96 well plates and serial 3-fold dilutions performed. Samples were transferred to an assay plate, and when the assay was complete plates were read in an Envision plate reader. Data were analyzed and plotted using Prism software.

To measure the formation of tetrameric NA a new, high-throughput TAMIFLU®-binding assay was developed. Oseltamivir-phosphate (TAMIFLU®) is a competitive inhibitor of the NA enzymatic activity. The oseltamivir substrate is specific to the enzymatic site of the tetrameric NA head. Oseltamivir-phosphate was used as a ligand to determine binding kinetics of recombinant NA proteins. Binding of recombinant NA to TAMIFLU® indicates the presence of an active site on the head region of the recombinant NA and was used to measure tetrameric NA concentration. Initially, using Tet-NA constructs, the TAMIFLU®-binding assay was validated by demonstrating that tetrameric NA of various influenza strains bind to TAMIFLU® in a dose-dependent manner. In contrast, monomeric NA ectodomain variants that are enzymatically inactive, do not bind TAMIFLU®. Thus, the level of recombinant NA binding in the TAMIFLU®-binding assay is proportional to the concentration of recombinant NA that are assembled as tetramers.

For the TAMIFLU®-binding assay, an oseltamivir-phosphate-biotin conjugate (5-10 μg/ml in 1×KB buffer (1% BSA+0.02% Tween in PBS)) was captured on the surface of streptavidin-coated biosensors, and the binding kinetics of neuraminidase to oseltamivir-phosphate was measured using the Bio-Layer Interferometry (BLI) technique on an Octet instrument (ForteBio, Molecular Devices, LLC, Fremont, California). The biosensors were dipped into wells containing serial 2-fold dilutions of a sample of recombinant NA (0.16-10 ug/ml in 1×KB). Any change in the number of molecules bound causes a measured shift in the pattern at the detector.

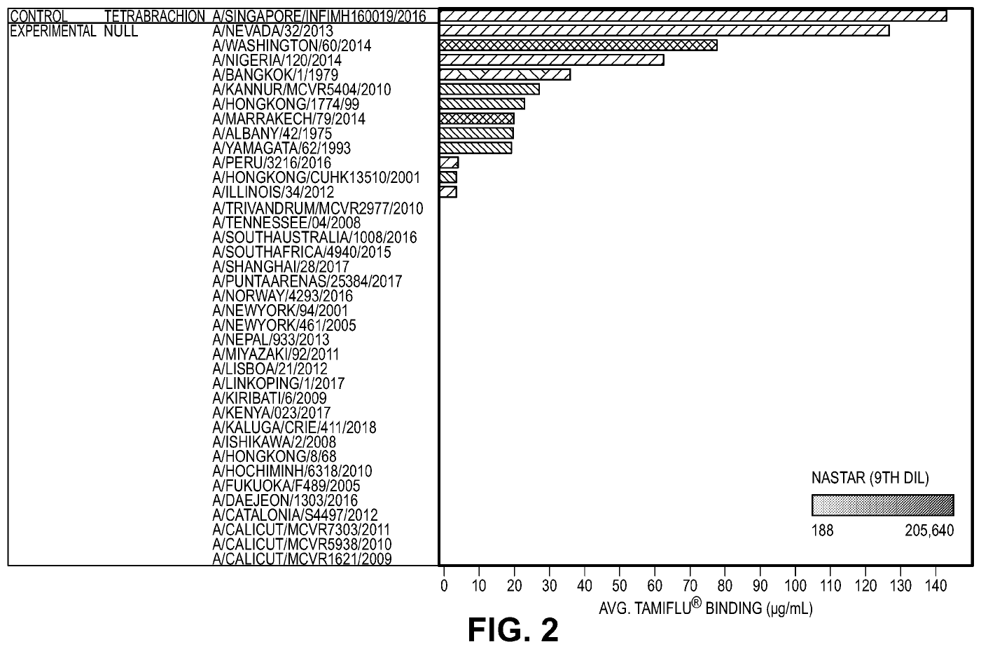

As shown in FIG. 2, a number of the 37 dTM75 constructs had substantial NA activity and/or TAMIFLU® binding (tetramer formation), including, for example, dTM75 constructs from the following strains: A/Nevada/32/2013, A/Washington/60/2014, A/Nigeria/120/2014, A/Bangkok/1/1979, A/Kannur/MCVR5404/2010, A/HongKong/1774/99, A/Marrakech/79/2014, A/Albany/42/1975, A/Yamagata/62/1993, A/Peru/3216/2016, A/HongKong/CUHK13510/2001, and A/Illinois/34/2012. A modified A/Singapore/INFIMH160019/2016 NA comprising a full-length, heterologous tetrabrachion tetramerization domain was used as a positive control. Although the dTM75 construct does not form active tetramers in all influenza subtype 2 strains tested, the TAMIFLU®-binding assay provides a convenient, high-throughput screening assay that can be used to identify which truncated N2 molecules form soluble tetramers when expressed in cells.

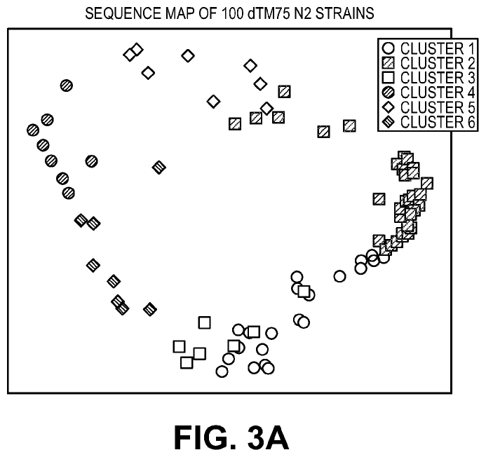

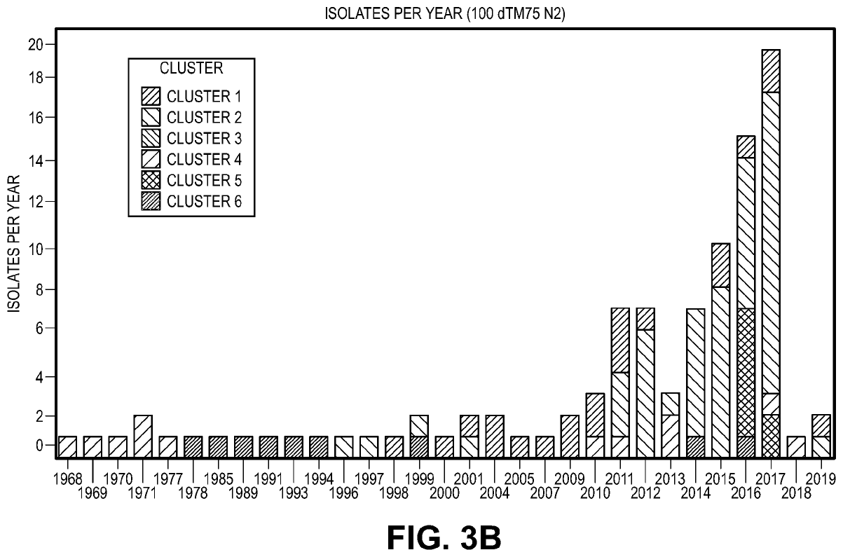

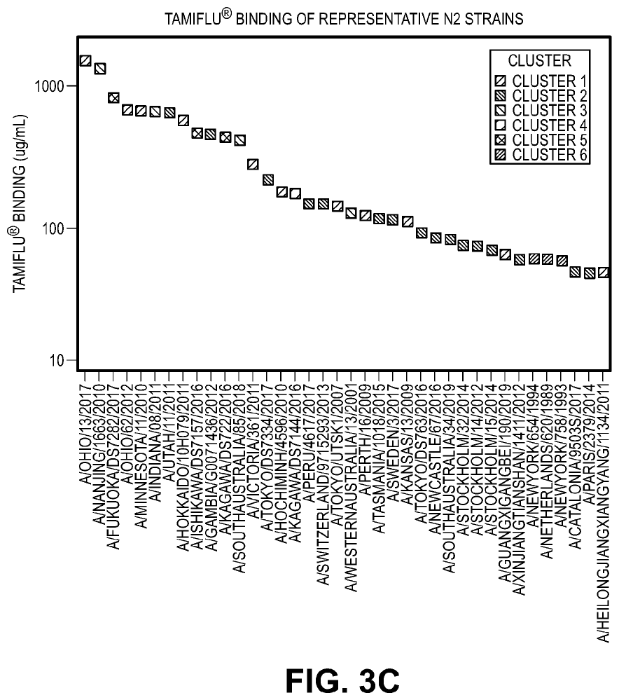

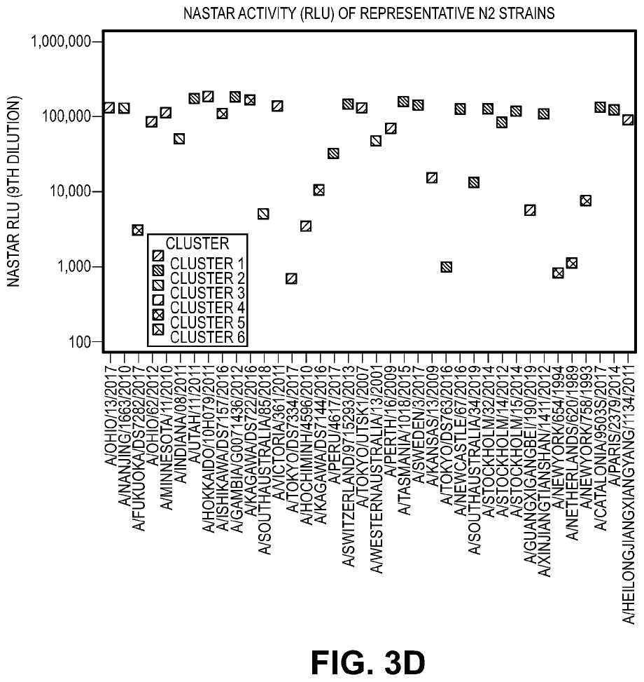

A second high-throughput screen was conducted for dTM75 constructs from about 100 additional subtype 2 influenza strains. The partial results are shown in FIGS. 3C-D. When expressed in cells, the dTM75 construct from the A/PERTH/16/2009 (PERT09) strain forms about 70% tetramer. About 20% of the dTM75 constructs from the additional strains tested in the second high-throughput screen exhibited higher TAMIFLU® binding than the PERT09 dTM75 construct. About 38% of the strains tested had TAMIFLU® binding that was within 3-fold of the binding observed with the dTM75 construct from PERT09. See FIG. 3C. The additional strains that were tested but did not show TAMIFLU® binding within 3-fold of PERT09 were A/TEXAS/50/2012 (SEQ ID NO: 87), A/PORTOALEGRE/LACENRS2376/2014 (SEQ ID NO: 69), A/MEMPHIS/18/1978 (SEQ ID NO: 49), A/BELGIUM/4217/2015 (SEQ ID NO: 5), A/TAIWAN/1/1969 (SEQ ID NO: 81), A/SIENA/3/1991 (SEQ ID NO: 71), A/BRISBANE/273/2016 (SEQ ID NO: 7), A/ONTARIO/RV3236/2016 (SEQ ID NO: 63), A/TEHRAN/996/2012 (SEQ ID NO: 85), A/ANKARA/2396/2015 (SEQ ID NO: 3), A/KANSAS/14/2017 (SEQ ID NO: 45), A/TEXAS/71/2017 (SEQ ID NO: 88), A/HONGKONG/4801/2014 (SEQ ID NO: 30), A/HONGKONG/3089/2017 (SEQ ID NO: 29), A/FUKUOKA/DS729/2016 (SEQ ID NO: 12), A/TOKUSHIMA/DS5288/2015 (SEQ ID NO: 89), A/HELSINKI/823/2013 (SEQ ID NO: 22), A/ALASKA/251/2015 (SEQ ID NO: 1), A/HATAY/4990/2016 (SEQ ID NO: 20), A/HANOI/ELI15597/2015 (SEQ ID NO: 19), A/MISSISSIPPI/1/1985 (SEQ ID NO: 51), A/INDIANA/18/2017 (SEQ ID NO: 36), A/POLAND/19B/2017 (SEQ ID NO: 68), A/TENNESSEE/18/2017 (SEQ ID NO: 86), A/HONGKONG/1774/1999 (SEQ ID NO: 28), A/DAKAR/14/2014 (SEQ ID NO: 10), A/SAUDIARABIA/21/1999 (SEQ ID NO: 70), A/GIFU/DS7388/2017 (SEQ ID NO: 15), A/NEWYORK/581/1997 (SEQ ID NO: 57), A/HELSINKI/941/2013 (SEQ ID NO: 23), A/ISHIKAWA/DS7294/2017 (SEQ ID NO: 39), A/GUANGDONGDUANZHOU/1227/2017 (SEQ ID NO: 16), A/AUCKLAND/5/1996 (SEQ ID NO: 4), A/ISHIKAWA/DS7215/2016 (SEQ ID NO: 38), A/WESTVIRGINIA/17/2012 (SEQ ID NO: 98), A/SINGAPORE/INFIMH160019/2016 (SEQ ID NO: 72), A/KAGAWA/DS7115/2016 (SEQ ID NO: 42), A/BILTHOVEN/21801/1971 (SEQ ID NO: 6), A/FUKUOKA/DS72/2016 (SEQ ID NO: 11), A/MORAMANGA/1907/2017 (SEQ ID NO: 52), A/HUNAN/01/2014 (SEQ ID NO: 33), A/WISCONSIN/16/2015 (SEQ ID NO: 100), A/CANBERRA/13/2015 (SEQ ID NO: 8), A/HONGKONG/107/1971 (SEQ ID NO: 27), A/LYON/1242/2000 (SEQ ID NO: 48), A/NAGANO/2153/2017 (SEQ ID NO: 53), A/NANJING/49/1977 (SEQ ID NO: 54), A/ALBANY/6/1970 (SEQ ID NO: 2), A/KAGAWA/DS769/2016 (SEQ ID NO: 41), A/TAIWAN/4183/2004 (SEQ ID NO: 83), A/KOREA/KUMCGR570/2011 (SEQ ID NO: 47), A/SYDNEY/24/2015 (SEQ ID NO: 80), A/KOREA/KUMCGR99/2011 (SEQ ID NO: 46), A/TOKYO/DS7277/2017 (SEQ ID NO: 91), A/HONGKONG/CUHK18194/1998 (SEQ ID NO: 31), A/GUNMA/DS7107/2016 (SEQ ID NO: 18), A/HONGKONG/8/1968 (SEQ ID NO: 26), A/TOKYO/DS7448/2017 (SEQ ID NO: 93), A/PERTH/61/2015 (SEQ ID NO: 65), A/TW/875/2004 (SEQ ID NO: 95), A/TAIWAN/2332/2001 (SEQ ID NO: 82), A/INDIA/C058671/2005 (SEQ ID NO: 34).

About 25% of the strains tested in the second high-throughput screen were below the level of detection. Thus, while a dTM75 construct did not form tetramers in all subtype 2 influenza strains that were tested, a majority of strains tested did form soluble NA tetramers when the dTM75 construct was expressed in cells, indicating that the dTM75 construct from subtype 2 influenza strains exhibits a high probability of yielding a soluble tetrameric NA. Further, the TAMIFLU®-binding assay provides a convenient way to quickly screen the truncated NA constructs to determine which ones form the desired tetrameric NA.

### Example 3. Serial Deletions of N2 Stalk to Identify Additional Soluble Designs

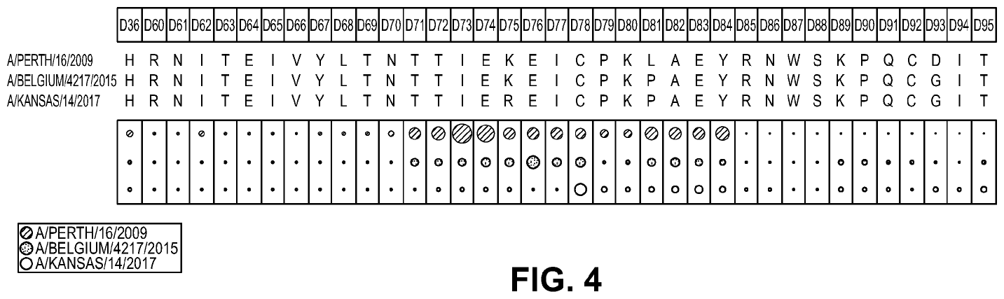

To assess whether other modified N2 proteins having smaller stalk truncations could yield soluble, tetrameric NA, a panel of constructs from PERT09 background was synthesized and screened for NA activity and TAMIFLU® binding as described above. One construct, dTM36 lacked the cytoplasmic domain and transmembrane domain (i.e., amino acids 1-35 of PERT09) but contained the entire stalk domain. See FIG. 1. Starting from amino acid 60 of the wild type PERT09 NA sequence and deleting one amino acid at a time, a panel of stalk truncation variants were created and tested for NA activity and TAMIFLU® binding and compared with results obtained for the PERT09 dTM75 variant. The PERT09 stalk truncation variants and the results of the high-throughput screen are shown in the table below. See also FIG. 4.

TABLE 2

| | | TAMIFLU ® | |
|---|---|---|---|
| Variant | NA Amino Acids | binding | NA |
| Name | Deleted | (μg/mL) | activity |
| dTM75 | 1-74 | 121 | 61312 |
| dTM74 | 1-73 | 227 | 169684 |
| dTM73 | 1-72 | 691 | 192036 |
| dTM72 | 1-71 | 159 | 143856 |
| dTM71 | 1-70 | 138 | 126276 |
| dTM70 | 1-69 | 40 | 70236 |
| dTM69 | 1-68 | 23 | 44656 |
| dTM68 | 1-67 | 13 | 24436 |
| dTM67 | 1-66 | 12 | 20680 |
| dTM66 | 1-65 | 4 | 6740 |
| dTM65 | 1-64 | 7 | 17024 |
| dTM64 | 1-63 | 6 | 15320 |
| dTM63 | 1-62 | 14 | 26696 |
| dTM62 | 1-61 | 20 | 35344 |
| dTM61 | 1-60 | 5 | 17788 |
| dTM60 | 1-59 | 5 | 14592 |
| dTM36 | 1-35 | 31 | 80396 |

The high-throughput screening results show that one truncation, dTM73, had significantly higher TAMIFLU® binding activities than the dTM75 control. Other designs had similar values to dTM75, including dTM74, dTM72, and dTM71. These results suggested that it is possible to use other stalk truncation variants of comparable size to dTM75 to successfully produce soluble tetrameric N2.

Serial deletion analysis was performed for two additional subtype 2 influenza strains and extended past amino acid 74 of the stalk region and into the NA head region, which is believed to start around amino acid 83 of the N2 sequence. The two additional strains were A/BELGIUM/4217/2015 (BELG15) and A/KANSAS/14/2017 (KANS17). Sequential deletion mutants, starting from amino acid 60 and extending through amino acid 90 for PERT09, BELG15, and KANS17 were synthesized and subjected to high-throughput screening as described above. Truncated N2 stalk variants lacking the entire stalk region formed soluble tetrameric NA in all three strains tested. From this screening assay, it was also shown that stalk deletions extending into the putative head region were still able to form tetrameric NA. While truncated variants lacking up to the first 84 amino acids of the N2 protein were able to form tetramers, extending the truncation beyond amino acid 84 did not result in the formation of soluble, tetrameric NA in the three N2 influenza strains tested. The screening analysis also showed that among all three strains tested, soluble tetrameric NA could be formed by truncated stalk variants of variable length, in which amino acid 36 to about at least amino acid 70-84 of the N2 stalk region had been deleted. For example, for PERT09, BELG15, and KANS17, a number of different stalk truncated variants formed tetrameric NA, with the dTM73 and dTM74 constructs showing the highest amount of TAMIFLU® binding for PERT09, the dTM76, dTM78, and dTM82 constructs showing the highest amount of TAMIFLU® binding for BELG15, and the dTM78 and dTM83 constructs yielding the highest amount of TAMIFLU® binding for KANS17. The results are shown in FIG. 4.

### Example 4. Large-Scale Production and Purification of dTM75

The dTM75 constructs from five subtype 2 influenza strains: PERT09, BELG15, KANS17, A/Peru/4617/2017 (PERU17), and A/Texas/71/2017 (TEX17) were expressed and purified at a large scale (typically 200 ml) for detailed characterization of yields, enzymatic activity and conformation. Recombinant NA constructs from each of these strains containing a full-length, heterologous tetrabrachion tetramerization domain (“tetNA”), as described above, were also expressed and purified at large scale.

Briefly, CHO-S cells were transfected with plasmids encoding the dTM75 constructs at 1 mg/mL. Transfections were performed using Gibco ExpiCHO transfection reagents (ThermoFisher Scientific, Waltham, MA). Cells were fed and temperature shifted from 36.5° to 32° C. one day after transfection. On day 4 post transfection cells were centrifuged at 10,500×g for 30 minutes and supernatant was filtered through a 0.2 μm vacuum filter. Clarified supernatants were purified by immobilized metal ion chromatography (IMAC) using the 6His tag (SEQ ID NO: 135) incorporated into the dTM75 protein. HisTrap™ HP (Millipore Sigma, Burlington, MA) columns, which are prepacked with Ni Sepharose High Performance and designed for simple, one-step purification of histidine-tagged proteins, were equilibrated with 20 mM phosphate buffered saline (PBS) with 500 mM sodium chloride (NaCl). Clarified supernatant samples were loaded directly onto HisTrap™ HP (Millipore Sigma, Burlington, MA) columns (5 mL column/200 mL supernatant). The column was washed with equilibration buffer and the target protein was eluted using a linear gradient of elution buffer 20 mM PBS with 500 mM NaCl and 1M imidazole buffer (0-100% elution buffer over 10 column volumes). This initial Ni-affinity chromatography step captures both monomeric and tetrameric NA.

Total yields of purified dTM75 samples of BELG15, PERT09, PERU17, TEX17 and KANS17 were calculated by measuring A280 protein concentration with appropriate molar extinction coefficients multiplied by volume of each recovered pool. The pools refer to the elution peaks. KANS17 eluted in two peaks, with the larger peak representing monomeric NA and the other peak representing tetrameric NA. For the remaining dTM75 constructs, no distinguishable resolution between peaks was observed and, therefore, a single pool was collected, representing a mixture of monomeric and tetrameric NA. High total yields were observed for all dTM75 constructs, in the range of about 25-250 mg/L for BELG15, PERT09, PERU17, TEX17, and KANS17.

The samples obtained from the initial Ni-affinity chromatography step were subjected to Size Exclusion Chromatography with Multi-Angle Light Scattering (SEC-MALS) to determine purity and molecular weight of purified dTM75 pools and subjected to further characterization (thermostability, TAMIFLU® binding, and NA activity). For SEC-MALS, a TSK-GEL G4000 PWXL (7.8 mm×30 cm) column from Tosoh Bioscience was used. The mobile phase for the column was 1×PBS with 0.02% Sodium Azide, pH 7.4. Empower® (Waters Corporation, Milford, MA) software was used to integrate UV peak areas on the chromatogram. The purity of the sample was calculated based on the percentage of the specific peak area/the total peak areas. The molecular weight (MW) of protein in the peaks was determined by ASTRA (Wyatt Technologies, Santa Barbara, CA) software using light scattering signals with a concentration detector (RI or UV). Recombinant N2 monomer (ectodomain) has a MW of about 74 kD, while tetrameric NA formed from tet-NA constructs has a MW of about 262 kD, about 4 times higher than monomeric NA.

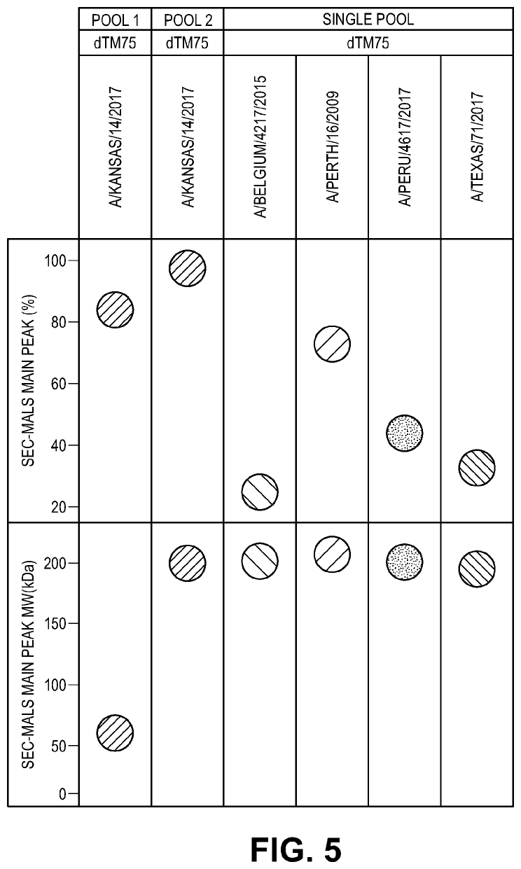

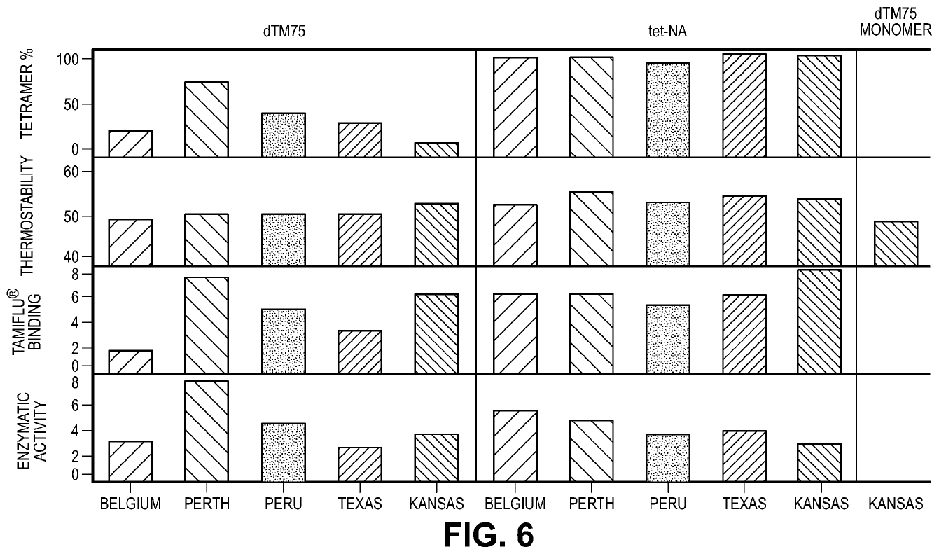

Results of the SEC-MALS analysis are shown in FIG. 5. For the dTM75 construct from KANS17, the MW of the pool 1 sample was about 60 kD, consistent with the expected size of a stalk truncated NA monomer, while the MW of pool 2 was about 210 kD, consistent with the expected size of a tetrameric NA formed by a stalk truncated NA (without a full-length tetrabrachion domain). FIG. 5 (bottom panel). The pool 2 fraction of the KANS17 dTM75 contained almost 100% tetrameric NA. FIG. 5 (top panel). For BELG15, PERT09, PERU17, and TEX17 a single pool was collected and analyzed by SEC-MALS. The calculated MW for each of these single pools was about 210 kD, indicating a tetrameric NA. FIG. 5 (bottom panel). The main peak of each of these single pools was also analyzed to determine the purity of the tetrameric NA in the single pool, which varied among the different strains tested. The dTM75 construct from PERT09 yielded about 70% tetrameric NA; the dTM75 construct from PERU17 yielded about 40-45% tetrameric NA; the dTM75 construct from TEX17 yielded about 30% tetrameric NA; and the dTM75 construct from BELG15 yielded about 20% tetrameric NA. FIG. 5 (top panel) and FIG. 6. The tetNA constructs from all five strains yielded about 90-100% tetramer. FIG. 6.

The purified tetrameric NA was subjected to further characterization. The tetrameric NA was tested for TAMIFLU® binding and enzymatic activity (MUNANA assay) as described above. All purified dTM75 constructs exhibited TAMIFLU® binding. FIG. 6. The dTM75 construct from PERT09 had the highest TAMIFLU® binding, comparable to PERT09 tet-NA TAMIFLU® binding, followed by KANS17 and PERU17, which also showed TAMIFLU® binding comparable to the PERU17 tet-NA construct. FIG. 6. All constructs that form tetrameric NA were enzymatically active. FIG. 6. Only the KANS17 pool 1 fraction (monomeric NA) was enzymatically inactive. FIG. 6.

The tetrameric NA was also tested for thermostability using the Applied Biosystems 7500 Fast Real-Time PCR instrument and Protein Thermal Shift™ (Thermo Fisher Scientific, Waltham, MA) software for calculating protein melting temperatures (Tm) by the Boltzmann or Derivative Curve methods. For the KANS17 dTM75 construct, the pool 2 (tetramer) purification fraction had higher Tm compared to the pool 1 (monomer) fraction, showing that tetrameric NA is a more stable molecule than monomeric NA. FIG. 6.

These large-scale production and purification experiments show that the truncated N2 stalk design can be applied to various subtype 2 influenza strains to successfully produce soluble, enzymatically active, tetrameric NA. Some strains, like PERT09 and PERU17, may produce a higher proportion of tetrameric NA. Some strains, like PERT09 and KANS17 may produce tetrameric NA with a higher binding affinity for TAMIFLU®. Yet, the design strategy can be consistently used to obtain tetrameric NA in subtype 2 influenza strains.

### Example 5. Large-Scale Production and Purification of Additional Truncated Stalk Variants

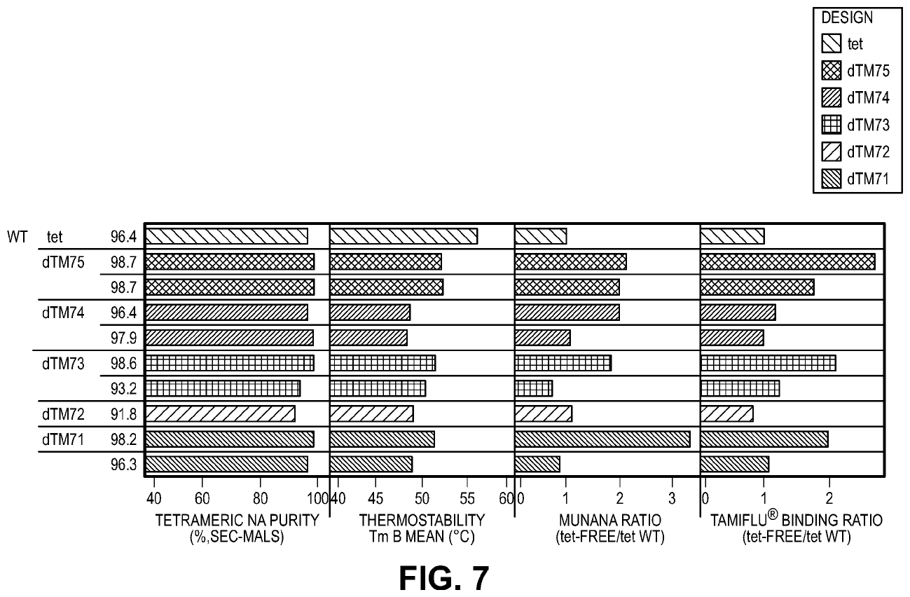

Additional truncated stalk variants in the PERT09 N2 background were expressed at large scale and purified by Ni-affinity purification as described above. Specifically, the following PERT09 constructs were synthesized, inserted into plasmids, transfected into CHO-S cells, and purified: dTM71, dTM72, dTM73, dTM74, and dTM75. All of the truncated stalk variants produced tetrameric NA that could be purified, confirming by SEC-MALS that other truncated stalk variants identified by the high-throughput TAMIFLU® binding assay form tetrameric NA and demonstrating that large quantities of highly purified tetrameric NA formed by these truncated stalk variants can be obtained following large-scale production. As shown in Table 3 below, the projected tetrameric yield of the truncated stalk variants ranged from about 50 mg/L to about 135 mg/L, and all of the constructs were enzymatically active, had TAMIFLU® binding activity, and thermostability of about 48-52 Tm. See also FIG. 7.

TABLE 3

| Matrix | | dTM75 | dTM73 | dTM74 | dTM72 | dTM71 |
|---|---|---|---|---|---|---|
| Tetramer purifiable | | Yes | Yes | Yes | Yes | Yes |
| tet-NA Yield Ratio | | 0.65-0.69 | 0.62 | 0.64 | 0.51 | 0.59 |
| (by % SEC-MALS)/total NA | | | | | | |
| Projected Tetramer yield (mg/L) | | 54-128 | 135 | 69 | 88 | 57 |
| Final Product Tetramer percentage | | 98-99% | 93-94% | 96-98% | 92% | 89-96% |
| (Native by SEC-MALS) | | | | | | |
| Covalent status (Denatured Non- | | Dimer | Dimer | Dimer | Dimer | Dimer |
| Reduced) by LabChip | | | | | | |
| Biological or | Enzymatic | 2.0-2.4 | 0.7-1.0 | 1.1-2.0 | 1.1 | 0.9 |
| Functional | Activity | | | | | |
| property* | TAMIFLU ®- | 1.8-2.4 | 1.0-1.2 | 1.0-1.2 | 0.8 | 1.1 |
| | Binding | | | | | |

| | | | | | |
|---|---|---|---|---|---|
| Thermostability | 51-52 | 49-50 | 48-49 | 49 | 48-49 |

### Example 6. Immunogenicity of dTM75 Construct in Mice

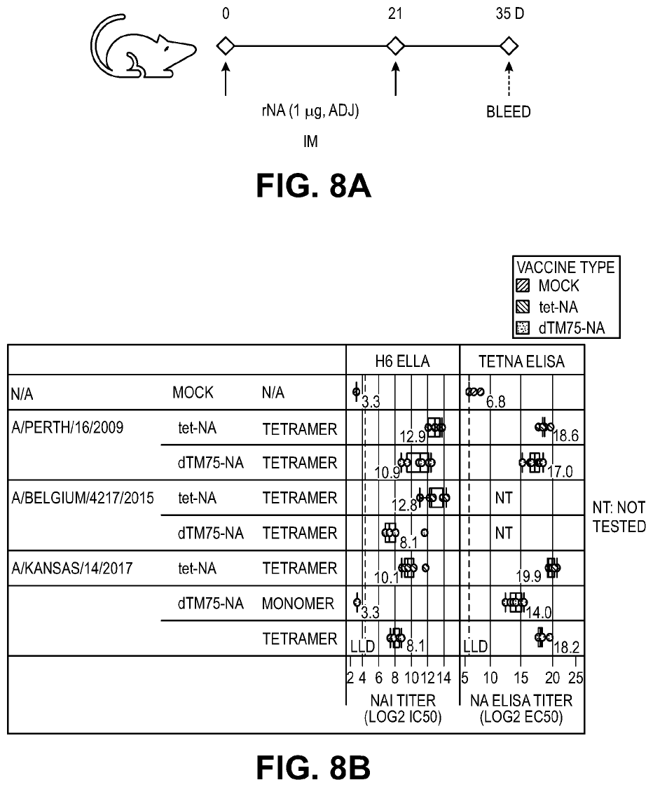

A naïve mouse model was used to assess immunogenicity of the recombinant, truncated stalk N2 variants. Sultana et al., Vaccine 29 (2011) 2601-2606; Eichelberger et al., Current Opinion in Immunology 2018, 53:38-44. Female 6-8 week-old BALB/c mice (n=6/group) were immunized twice intramuscularly (IM) at days 0 and 21 with Ni-affinity purified recombinant modified N2 at a dose of 1 microgram formulated with a squalene-in-water adjuvant (AF03). The experimental schedule is illustrated in FIG. 8A. Sera were collected 14 days after the second immunization.

Immunized mice were tested for the presence of NA-specific antibodies using a neuraminidase-specific Enzyme-Linked Immunosorbent Assay (NA ELISA). His-tagged tet-NAs derived from H3N2 strains of interest were immobilized on nickel-coated plates. After plates were washed, they were blocked with 5% Milk BLOT-QuickBlocker™ (G-BioSciences, St. Louis, MO) to eliminate non-specific binding. Next, the plates were incubated with serially diluted sera from the immunized mice to allow binding of anti-NA antibodies followed by incubation with HRP-linked Goat anti-mouse IgG-detecting antibody (ABCAM, Cambridge, UK) and introduction of a mixture containing TMB (3,3′,5,5′-tetramethylbenzidine) and hydrogen peroxide. Reactions were stopped by adding acidic stop buffer. Absorbance at 450 nm was read with the Plate reader SpectraMaxi3 (Molecular Devices, San Jose, CA). The OD450 values were approximated with a non-linear 4PL curve using GraphPad Prism (San Diego, CA) software and the 50% maximal effective concentration (EC50) was calculated.

Neuraminidase inhibitory antibody responses were measured against H6N2 reassortant viruses containing NA derived from the H3N2 strain of interest by Enzyme-Linked Lectin Assay (ELLA). Briefly, a H6N2 reassortant virus containing the NA derived from an H3N2 strain of interest was titrated in a fetuin-coated plate to determine the standard amount of virus that provides 70% of maximum NA enzymatic activity. Titration of NA inhibiting (NAI) antibodies present in the sera of immunized mice was achieved by performing serial dilutions of heat inactivated sera and adding to a standard amount of virus in a fetuin-coated plate. The serum-virus mixture was incubated overnight. The plate was washed and developed following incubation with peroxidase-conjugated PNA. Low or no signal relative to a virus control indicates inhibition of NA activity due to the presence of NA-specific antibodies. NAI titers were approximated with non-linear 4PL curve using GraphPad Prism software and the 50% maximal inhibitory concentration (IC50) was calculated.

Naïve BALB/c mice were immunized with a vaccine composition comprising a squalene-in-water adjuvant (AF03) and Ni-affinity purified dTM75 variants from KANS17, BELG15, and PERT09 or tet-NA positive controls from each strain according to the schedule illustrated in FIG. 8A. For KANS17 two vaccine compositions were administered, one containing monomeric dTM75 NA (obtained from the pool 1 eluate) and tetrameric dTM75 NA (obtained from the pool 2 eluate) eluates. As measured by ELISA, pool 2 KANS17 dTM75 elicited NA-specific IgG responses that were comparable in magnitude to the KANS17 tet-NA positive control. FIG. 8B. The monomeric NA from the pool 1 fraction of KANS17 dTM75 elicited lower ELISA titers as compared to the tetrameric pool 2 fraction. FIG. 8B. Similarly, the pool 2 KANS17 dTM75 fraction elicited much higher NAI responses than the pool 1 KANS17 dTM75 fraction, as determined by ELLA, albeit lower than compared to KANS17 tet-NA control. FIG. 8B.

The tetrameric NA formed by the PERT09 and BELG15 dTM75 constructs elicited high homologous ELISA titers, similar to the results obtained with the tetrameric dTM75 from KANS17. FIG. 8B. The total IgG NA-specific responses were comparable between all three dTM75 constructs tested and approached those of the tet-NA controls. Like the tetrameric dTM75 from KANS17, the tetrameric dTM75 from PERT09 and BELG15 also elicited NAI responses, albeit lower than NAI responses induced by tet-NA. FIG. 8B.

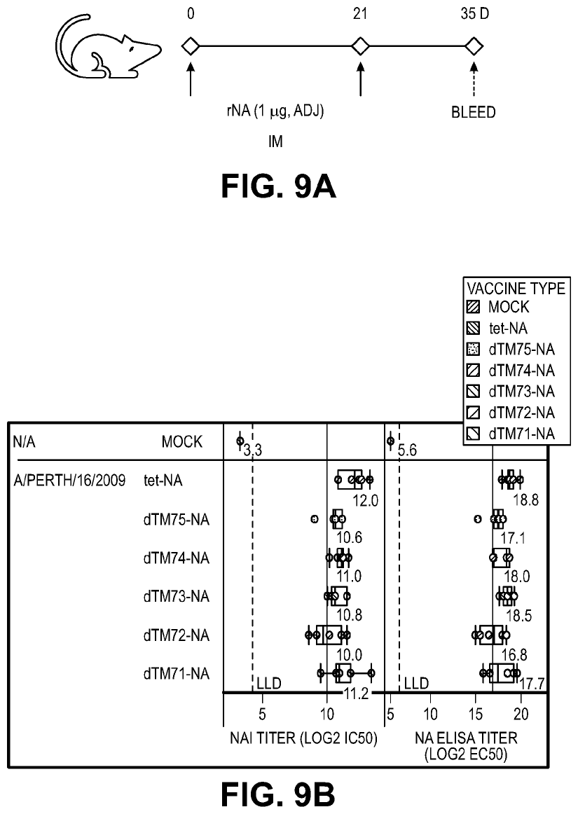

Next, the mouse immunogenicity studies were expanded to evaluate other N2 stalk truncated variants of varying length and compare them to the dTM75 construct. Specifically, the immunogenicity of the following PERT09 dTM variants were evaluated: dTM74, dTM73, dTM72, and dTM71 and compared to PERT09 dTM75 and tet-NA constructs. Naïve BALB/c mice were immunized with the dTM variant plus a squalene-in-water adjuvant (AF03) on days 0 and 21 or a tet-NA control plus adjuvant (AF03) according to the schedule illustrated in FIG. 9A.

NA-specific IgG responses were measured by ELISA on day 35 as described above and expressed as EC50 ELISA titers. NAI responses were measured against the H6N2 reassortant virus expressing homologous full-length NA by ELLA and expressed as IC50 NAI titers as described above. Individual animal titers were normalized (Log 2) and graphed as a box plot. Dashed line indicates assay's low limit of detection (LLD), while grey shaded area represents titers within 4-fold of the tet-NA control protein. As shown in FIG. 9B, immunogenicity of the PERT09 dTM75, dTM74, dTM73, dTM72, and dTM71 variants in naïve mice was comparable to the tet-NA control.

Overall, these mouse data show that tetrameric NA formed by a number of different truncated N2 stalk variants of various length and from different N2 strains induce NA-specific antibody responses in mice.

### Example 7. Immunogenicity of dTM75 Construct in Pre-Immune Ferrets

Ferrets have been previously used to assess immunogenicity of influenza neuraminidase, although most studies have focused on naïve responses to vaccines or response to natural infection (Bosch et al. J. Virol. October 2010, p. 10366-10374). Here, instead of using naïve or naturally infected ferrets, a new immunogenicity model was developed in which ferrets are pre-immunized by the intranasal route with an influenza H1N2 reassortant virus expressing a wild type NA of interest, e.g., the wild type NA from one of the subtype 2 influenza strains used to develop the truncated, stalk variants, such as the KANS17 H3N2 strain. This pre-immune ferret model measures the ability of a recombinant NA antigen to recall or boost responses elicited by prior virus infection.

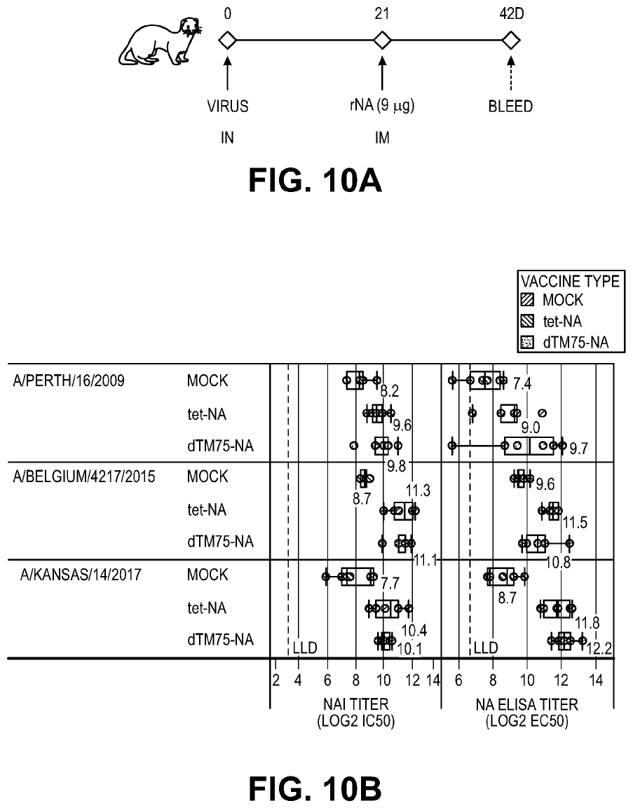

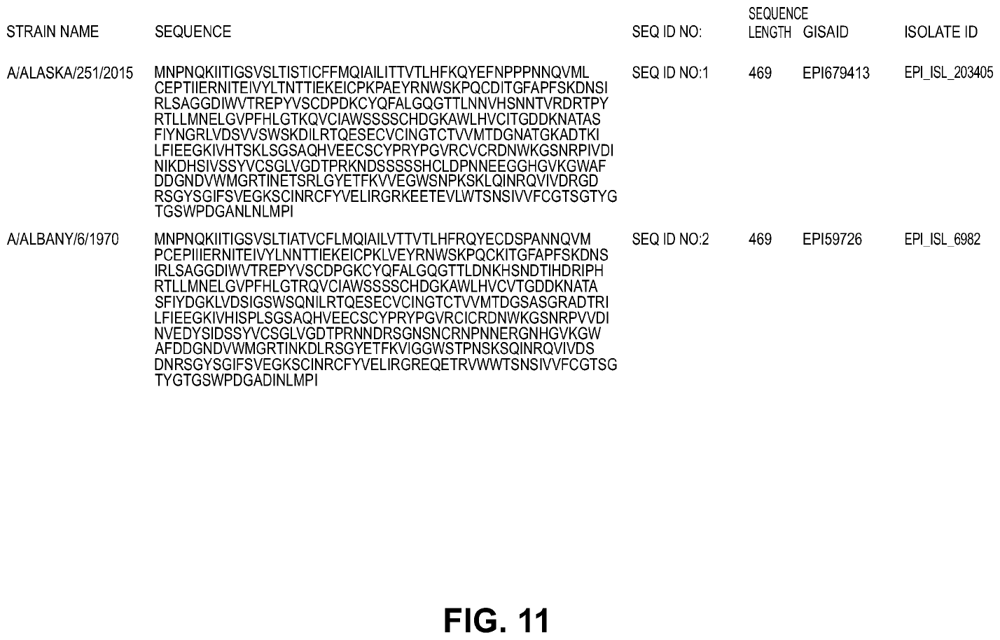

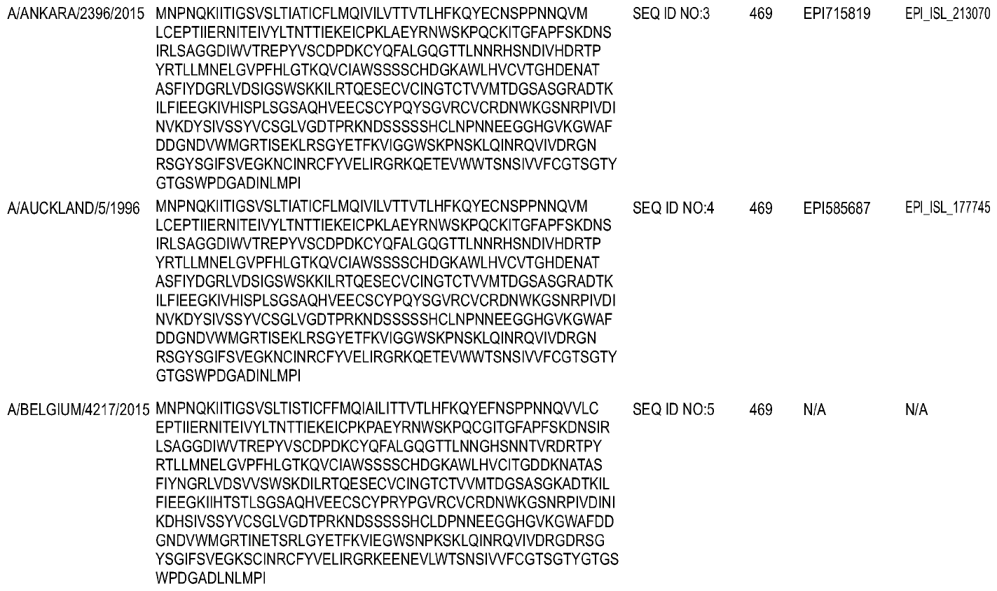

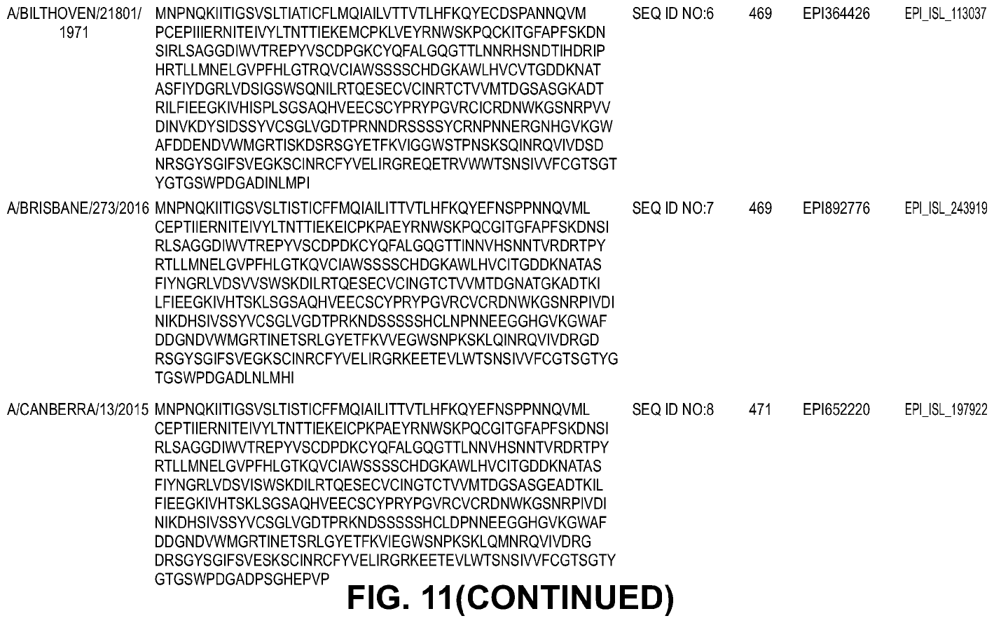

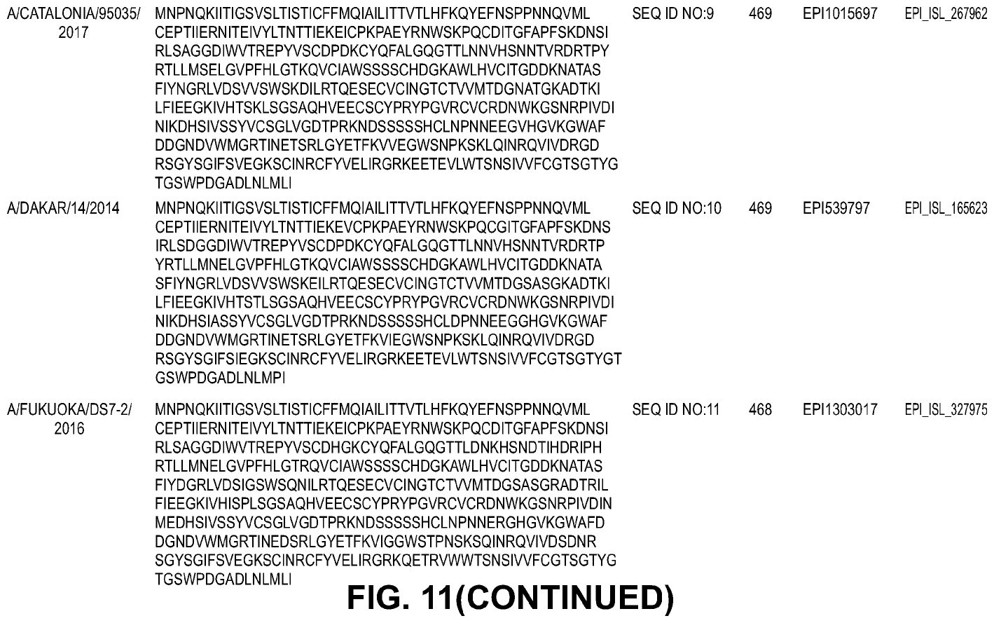

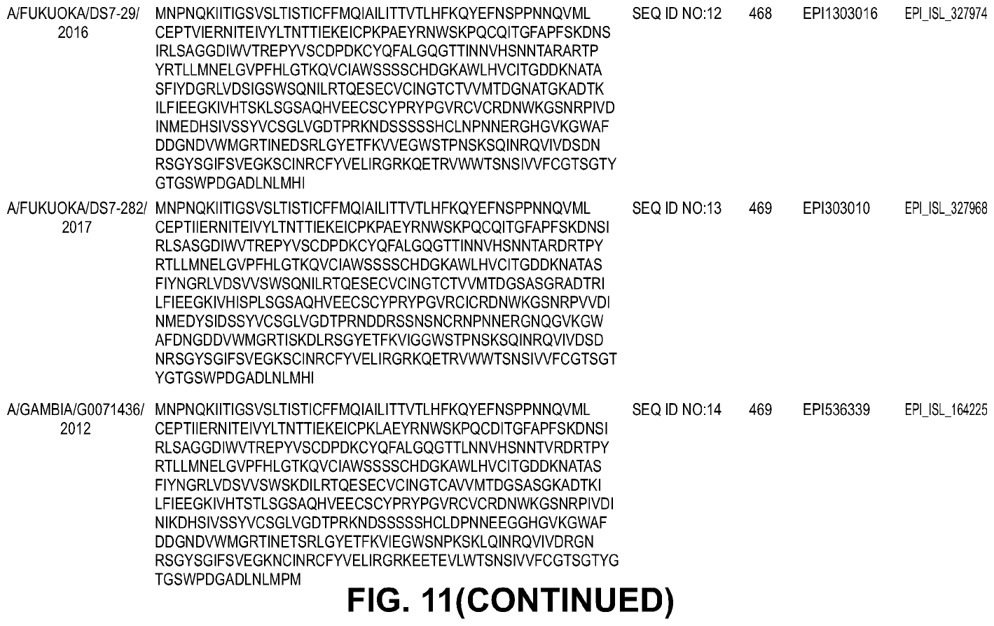

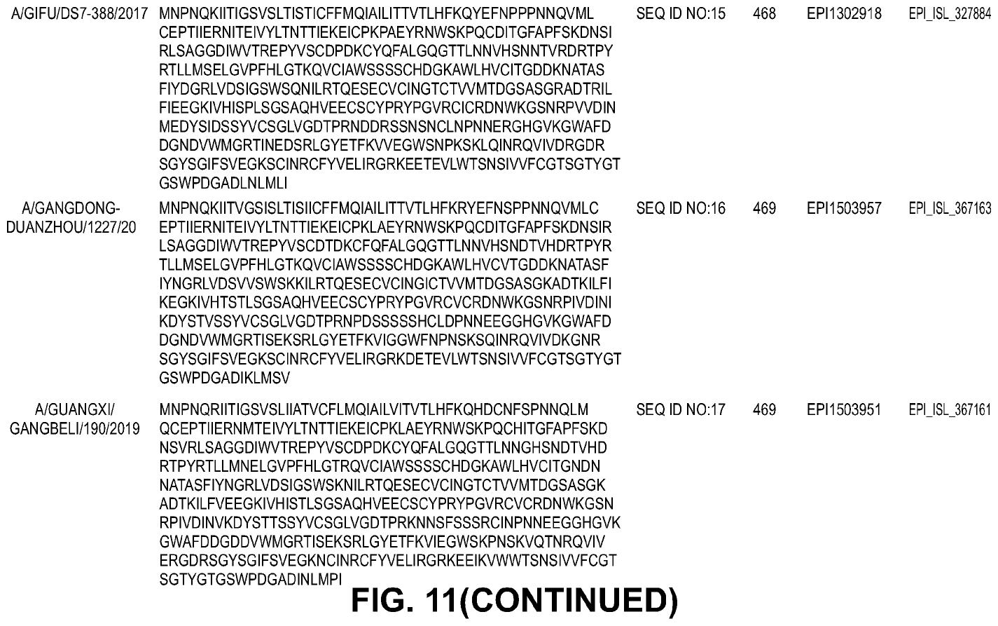

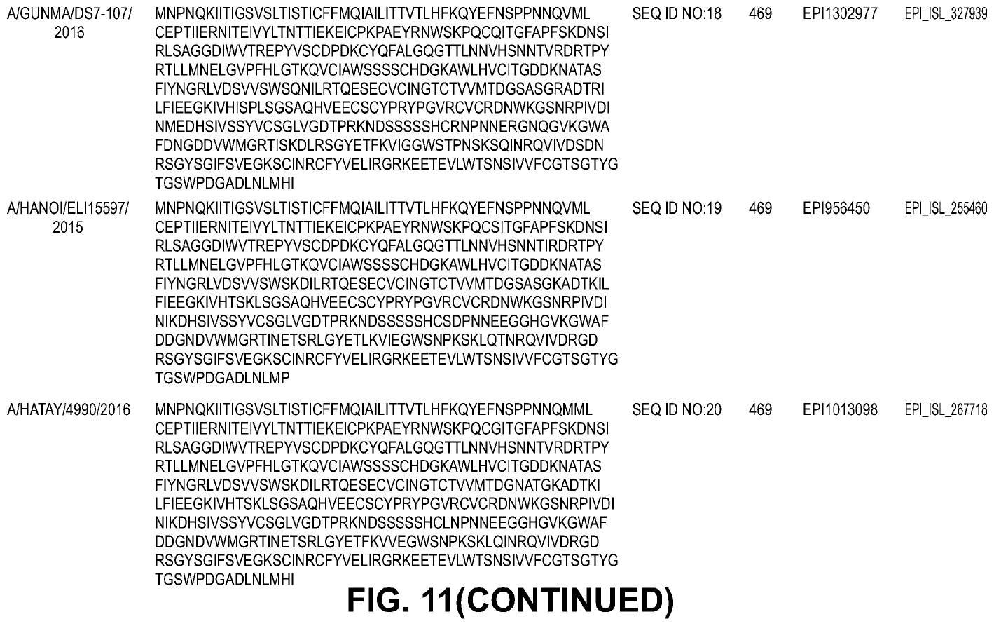

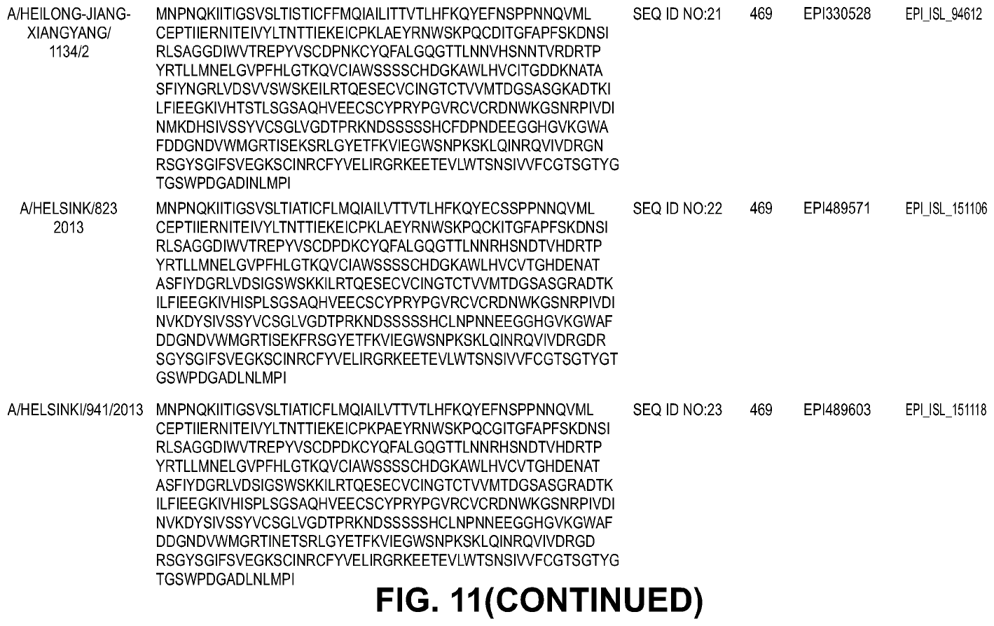

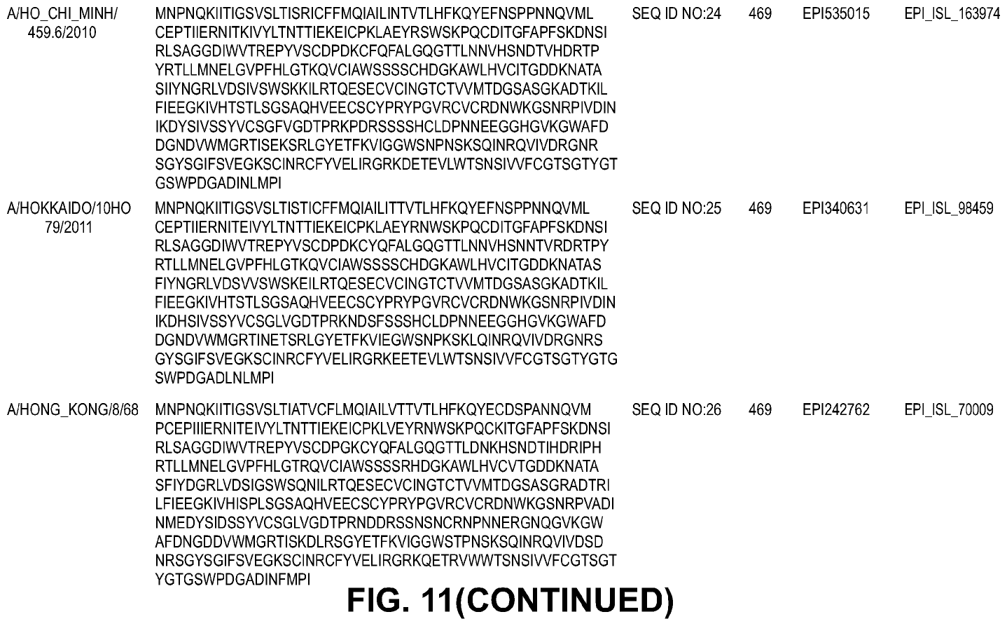

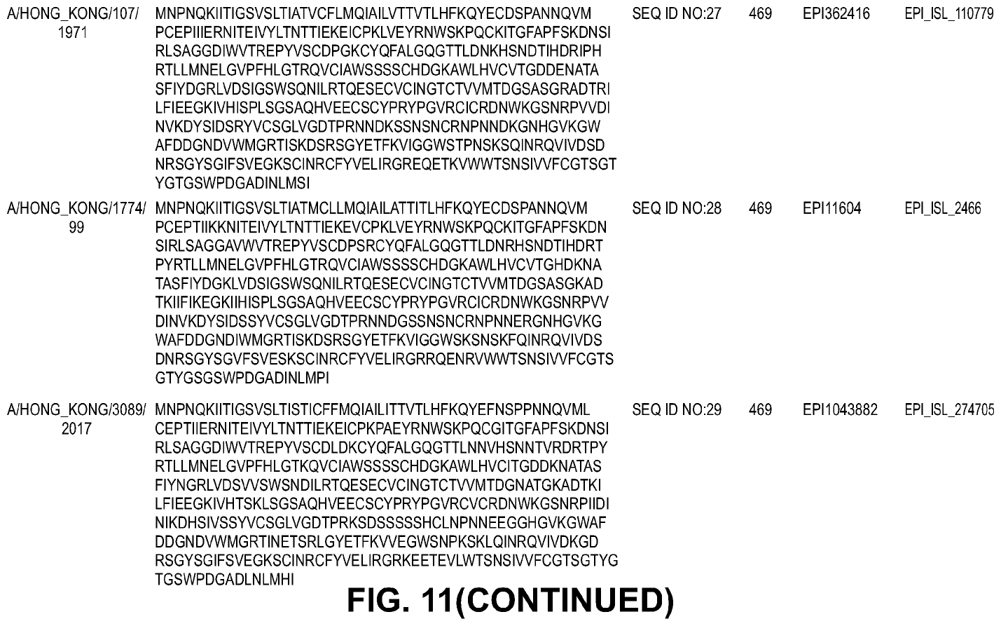

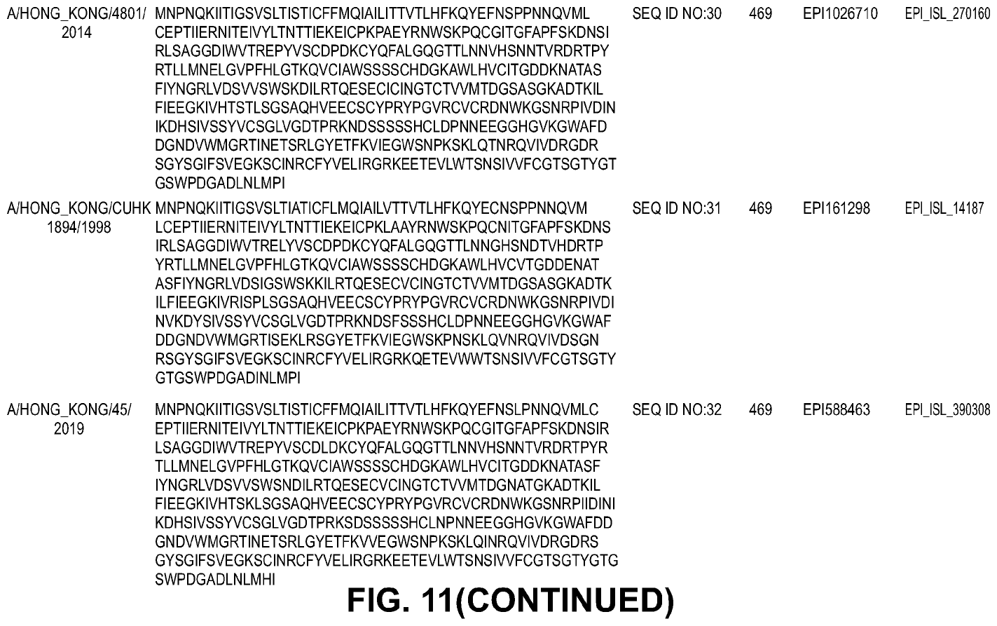

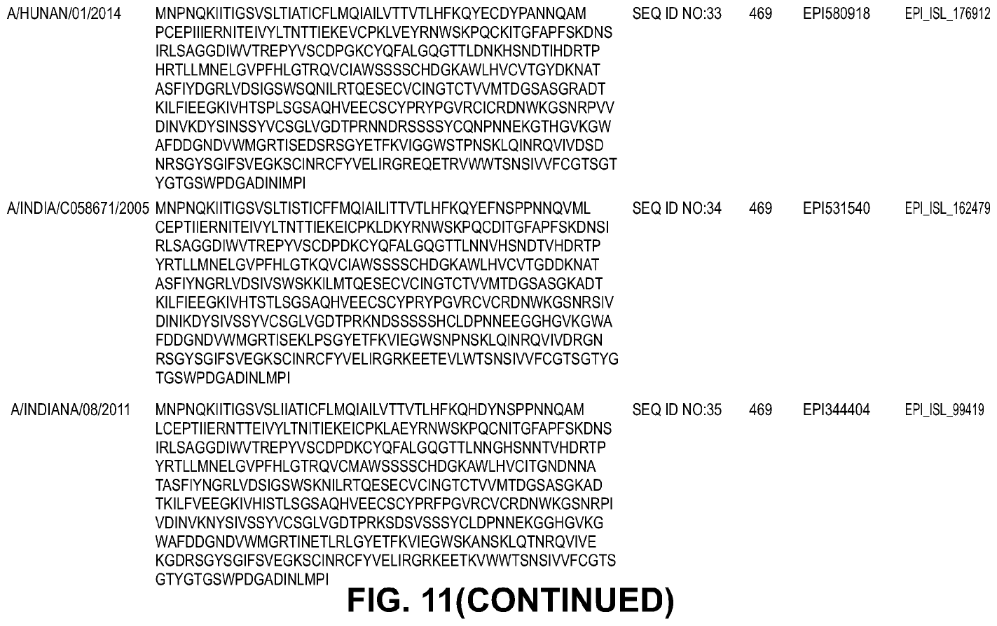

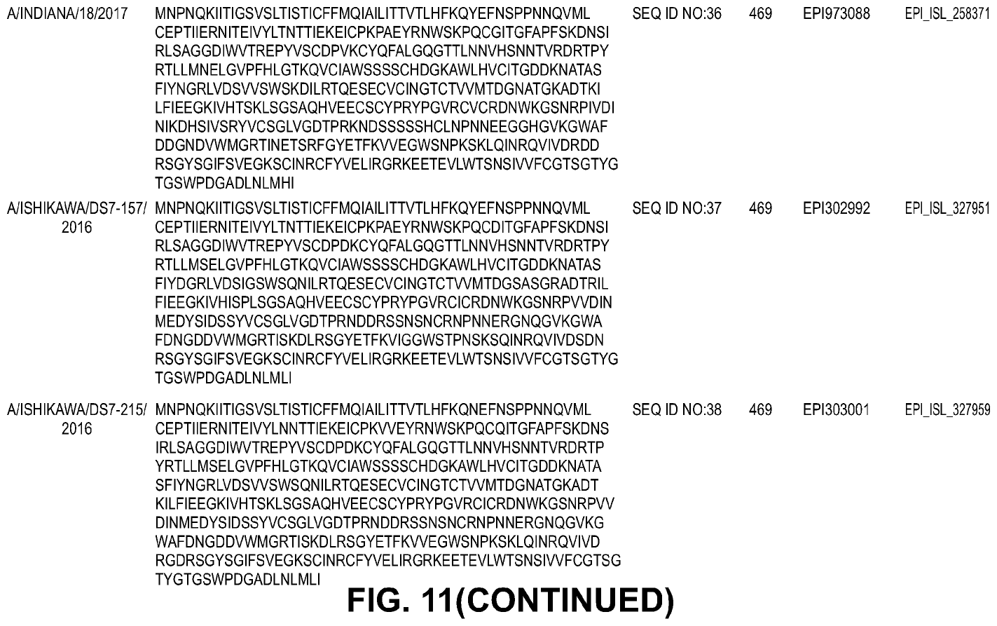

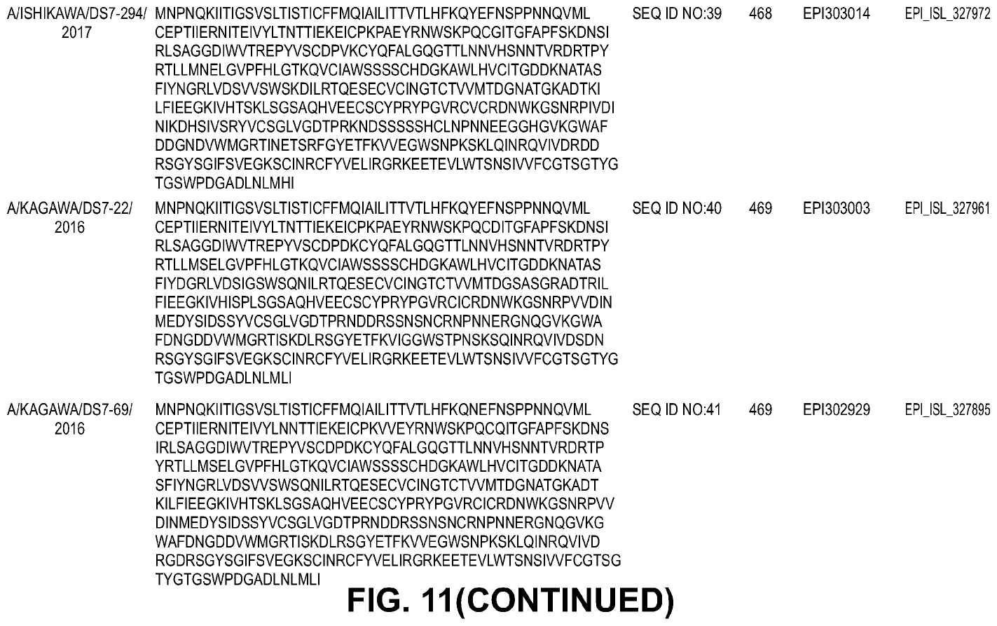

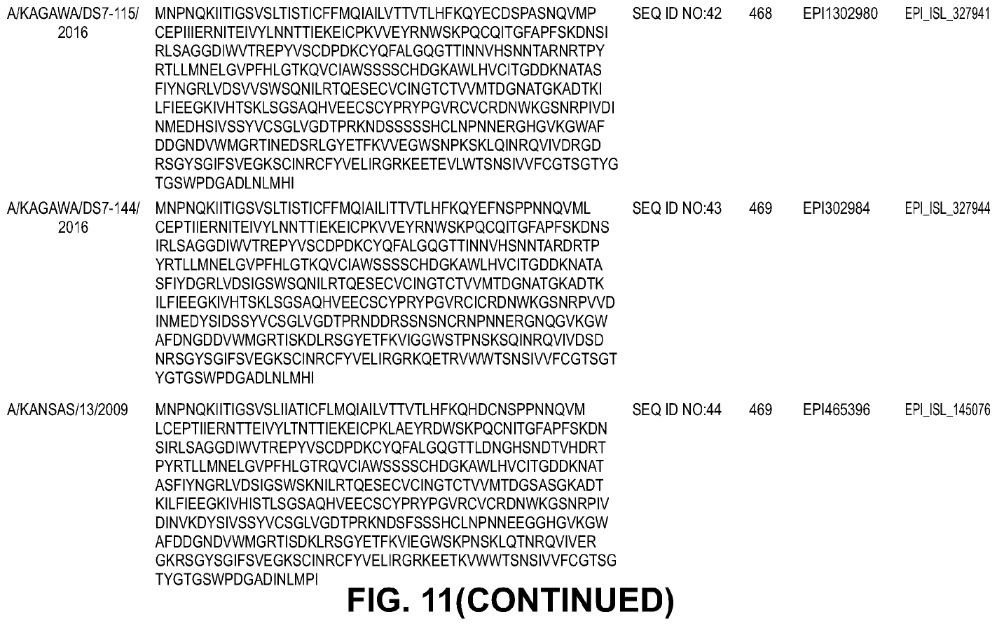

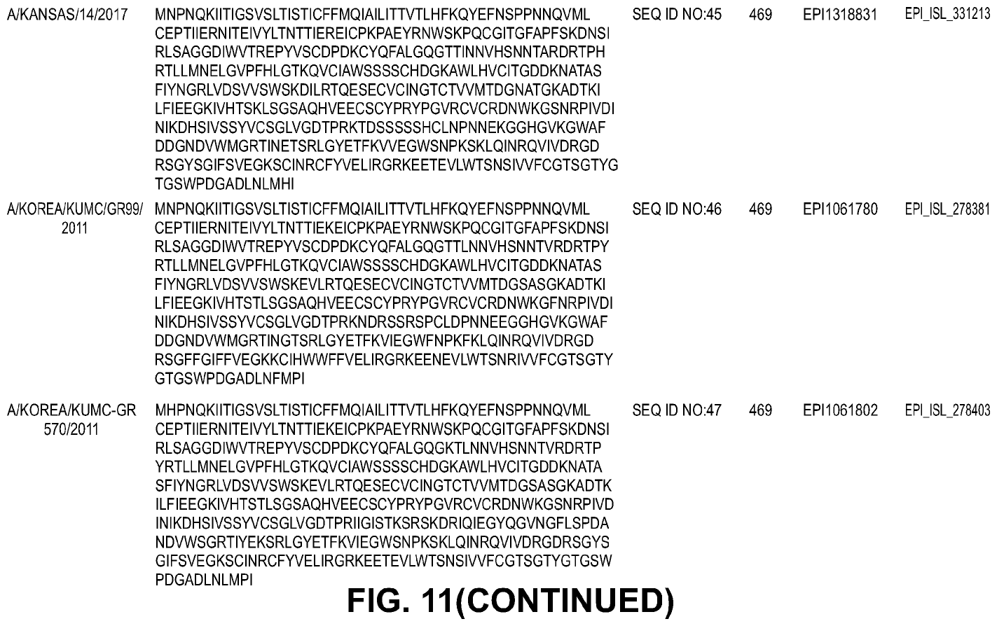

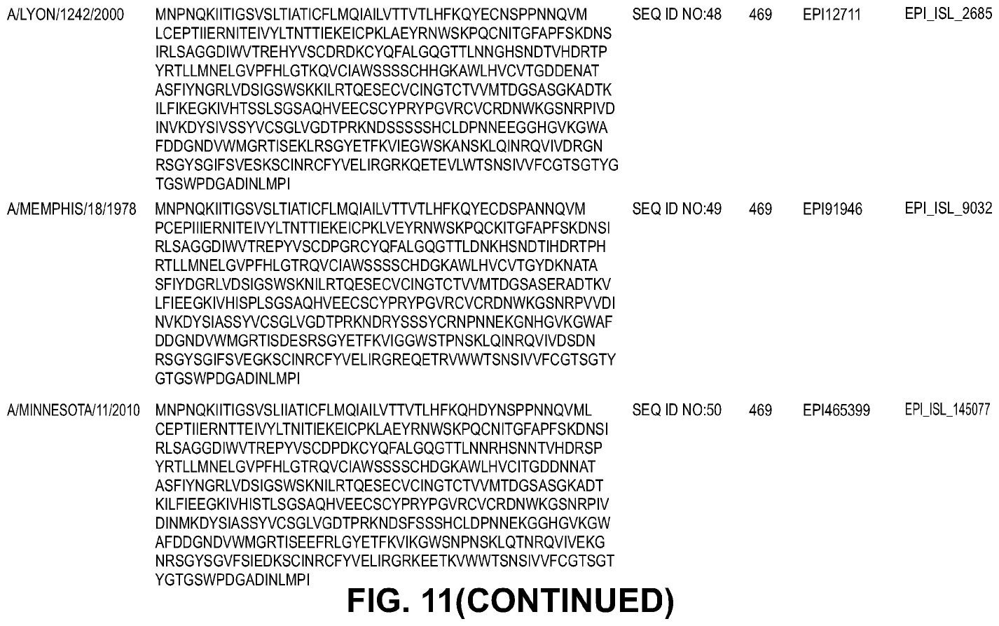

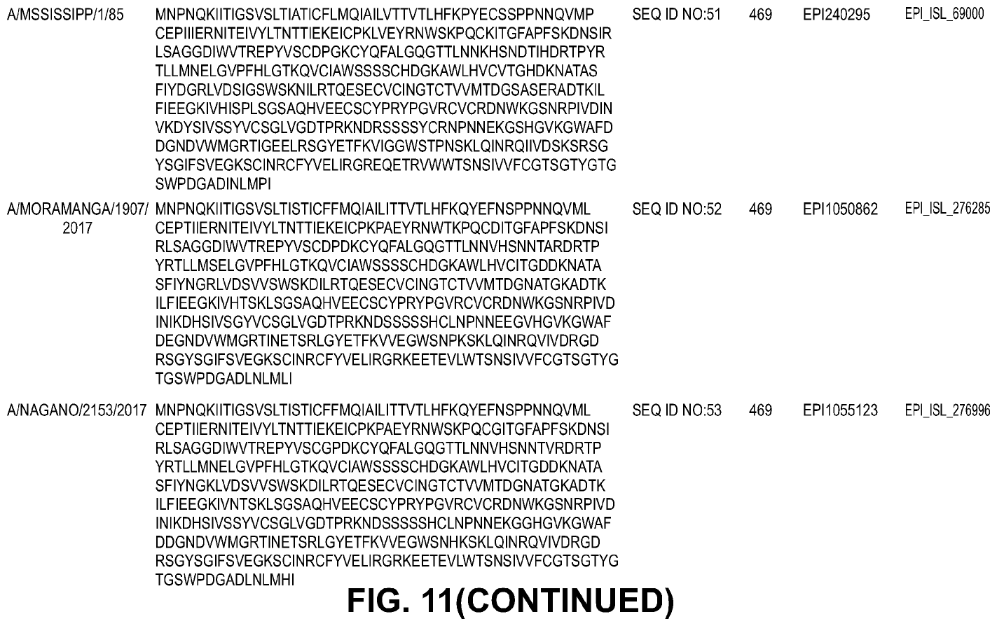

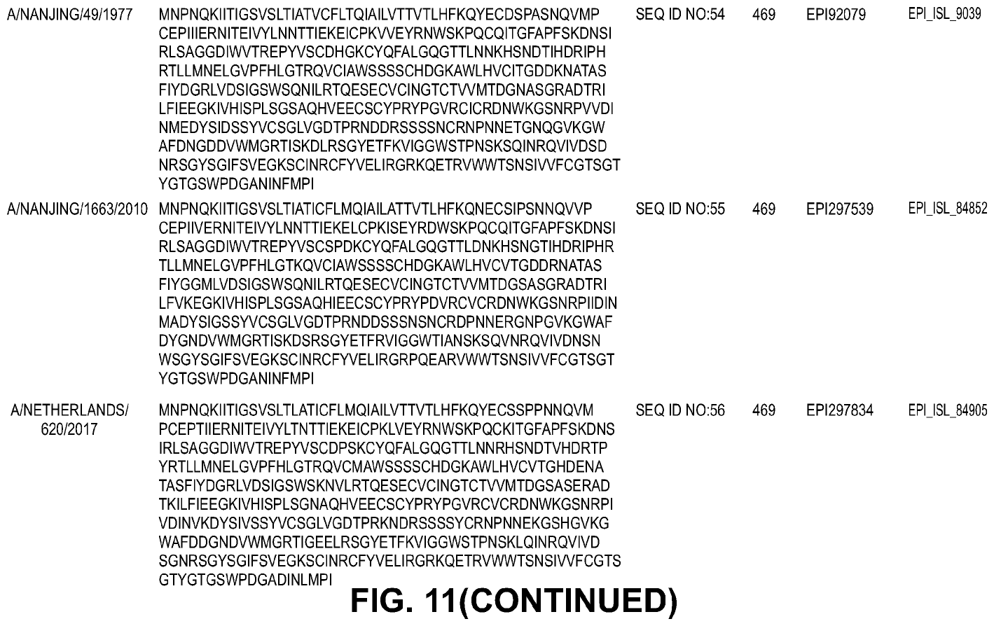

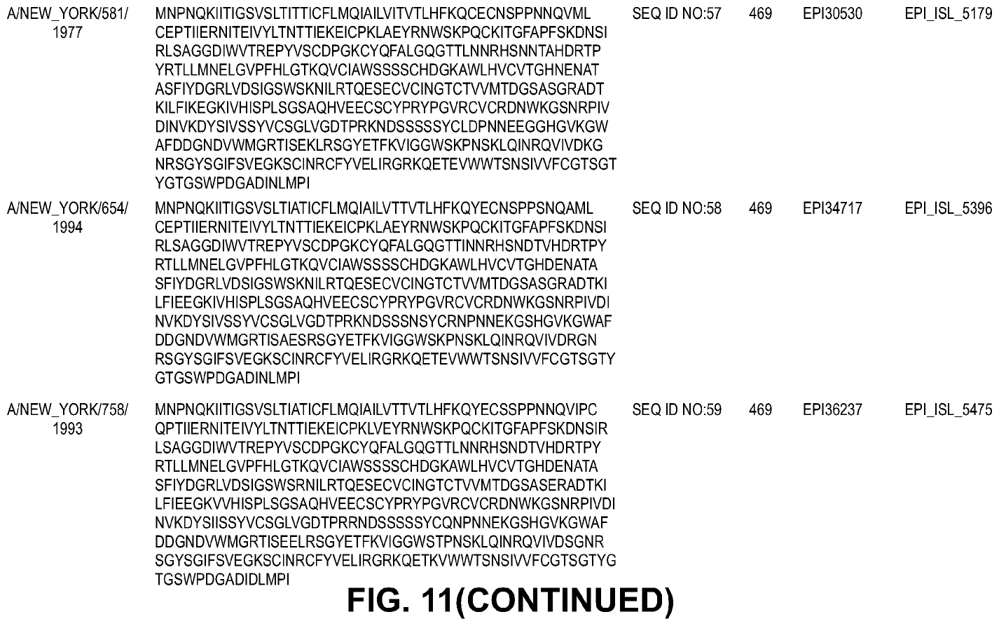

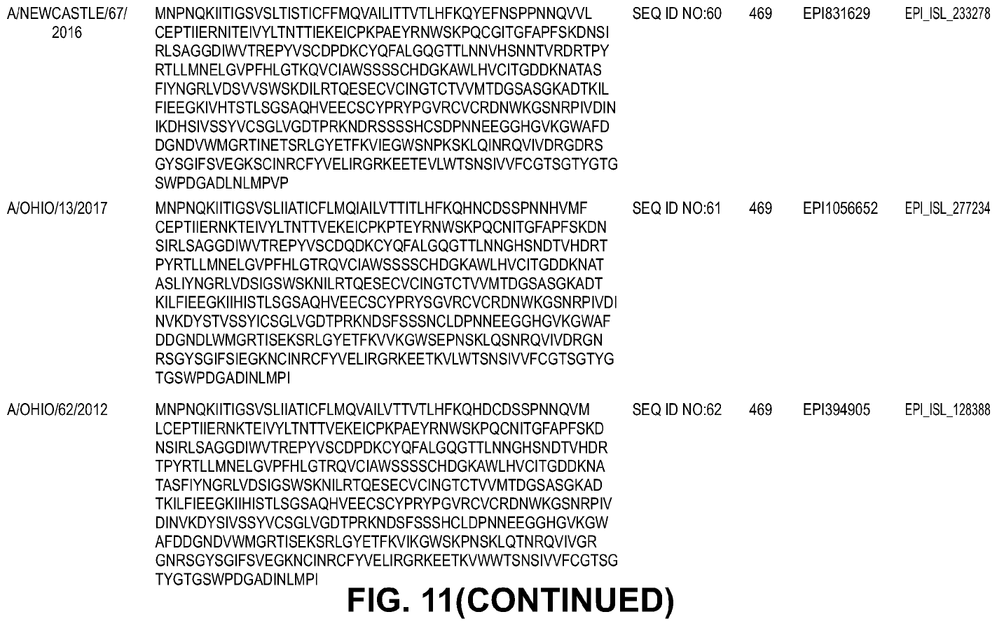

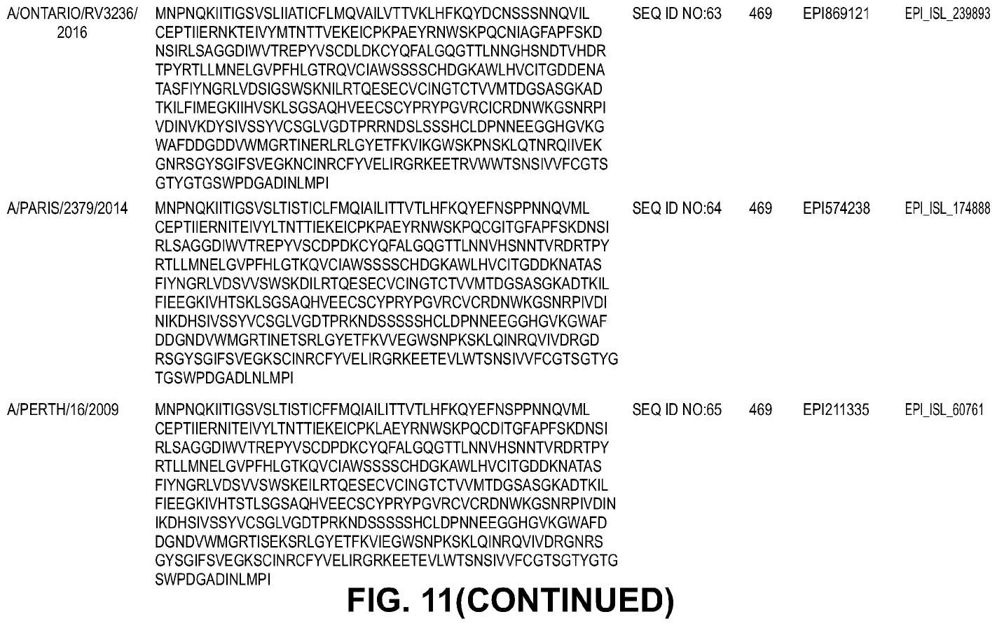

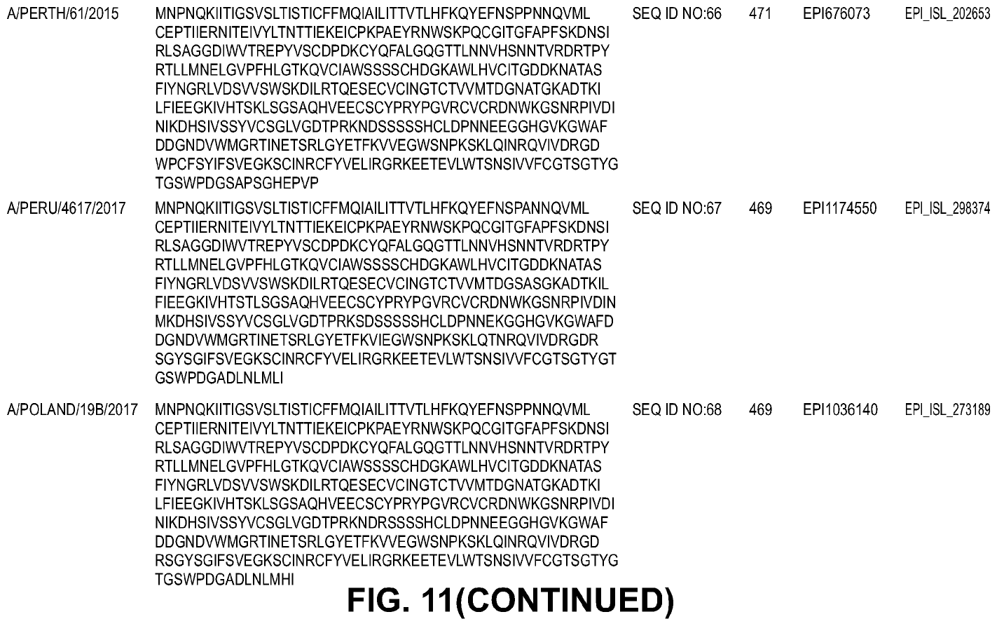

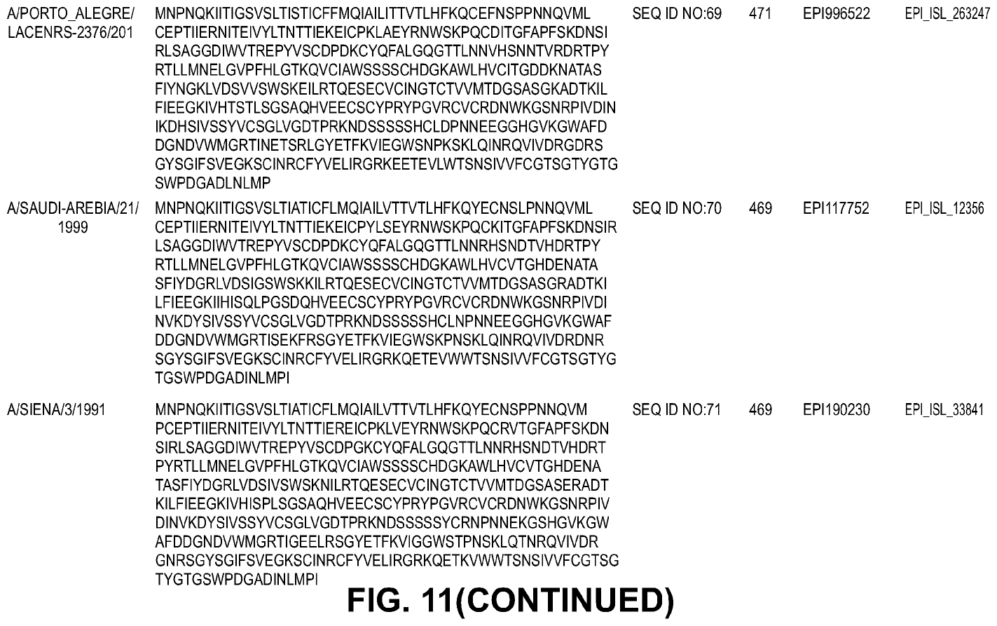

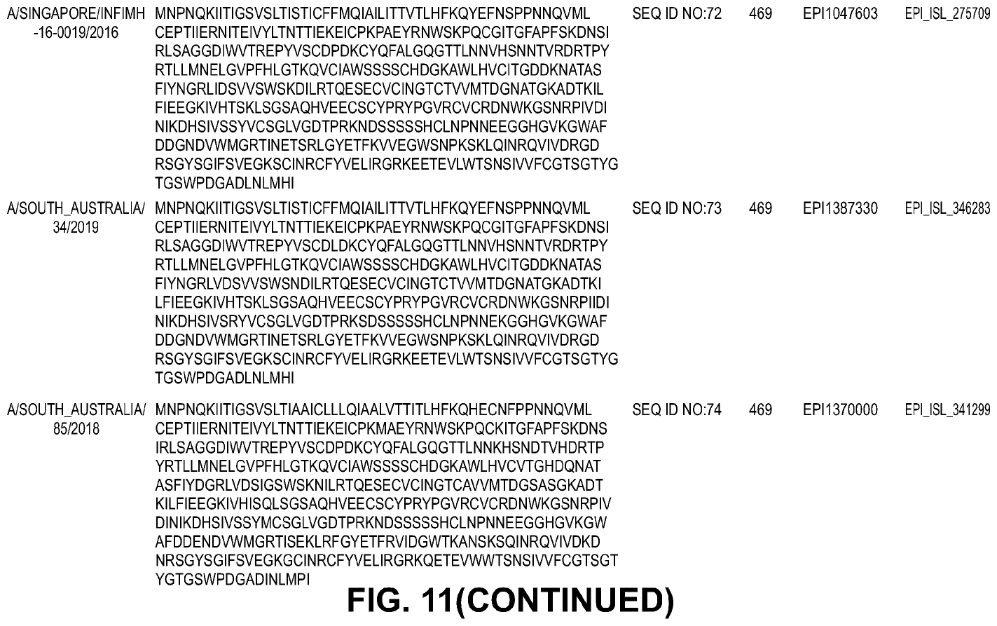

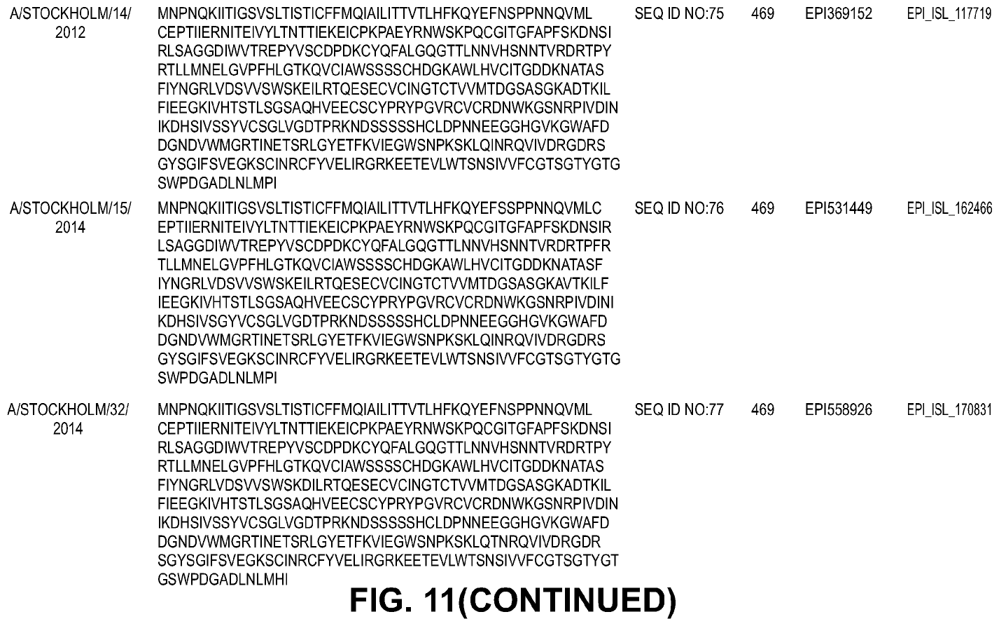

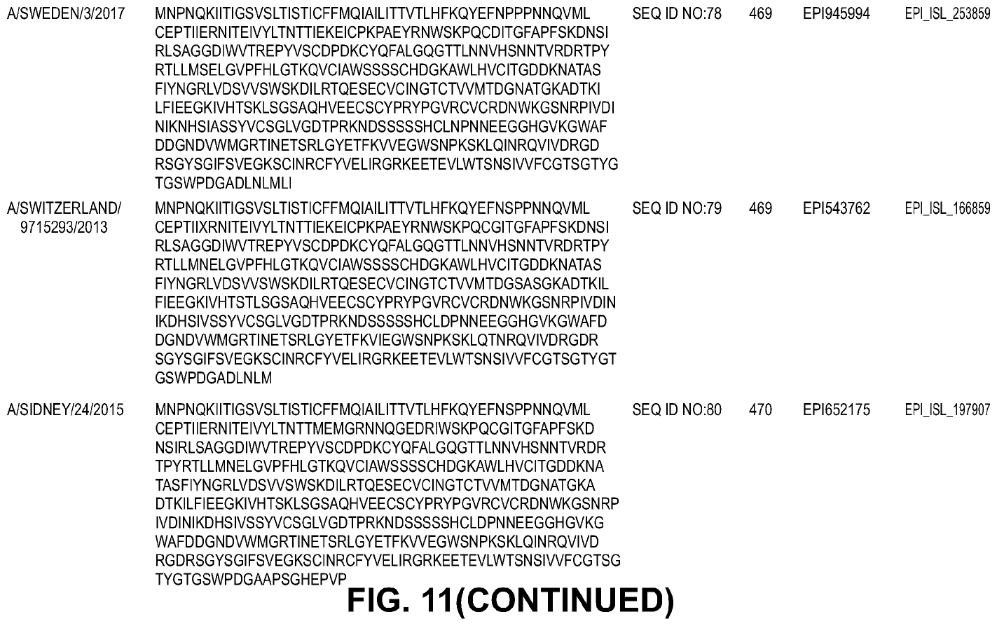

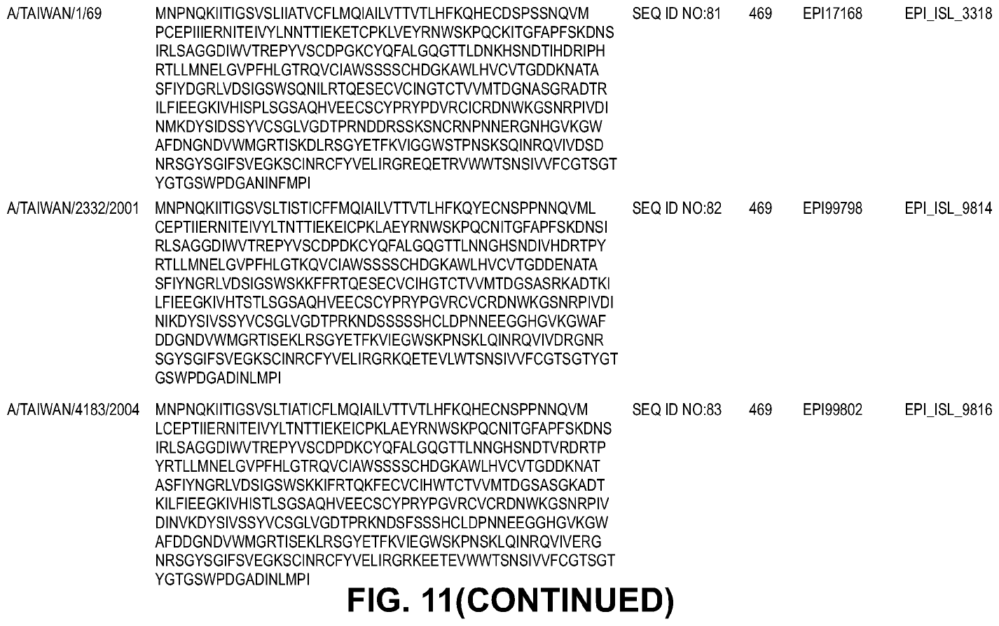

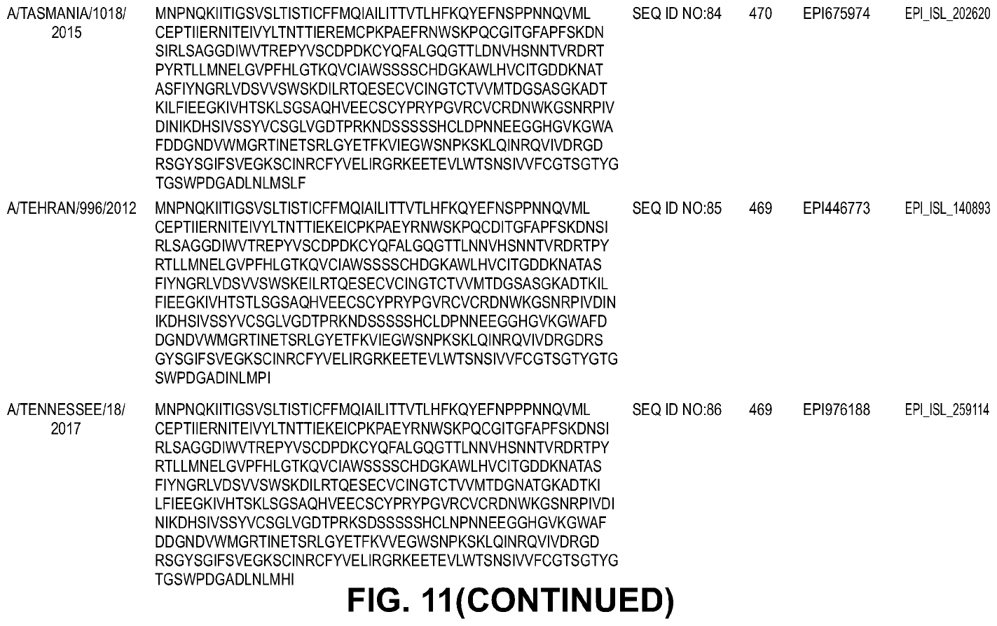

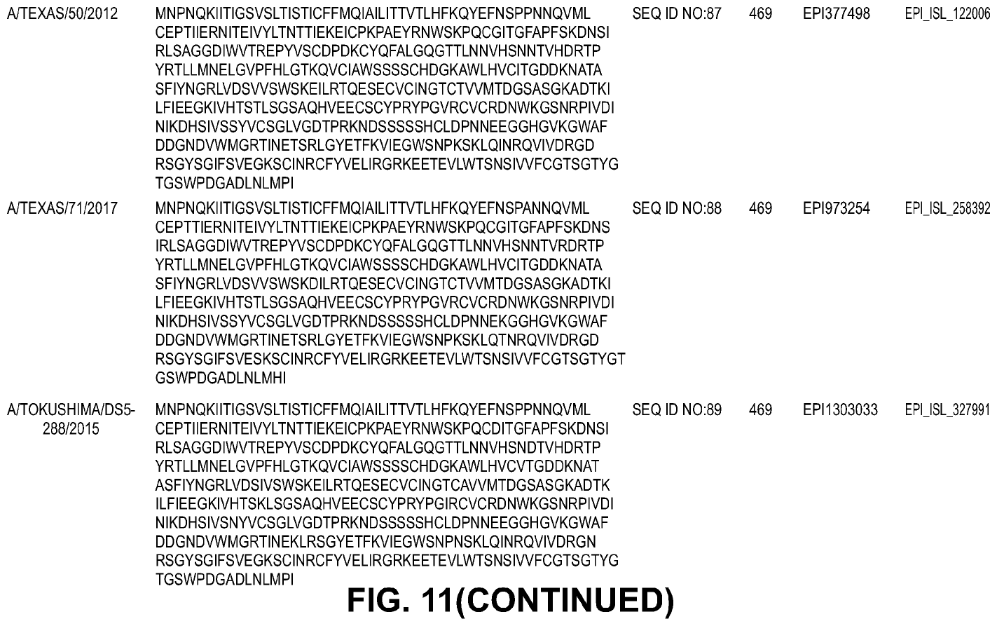

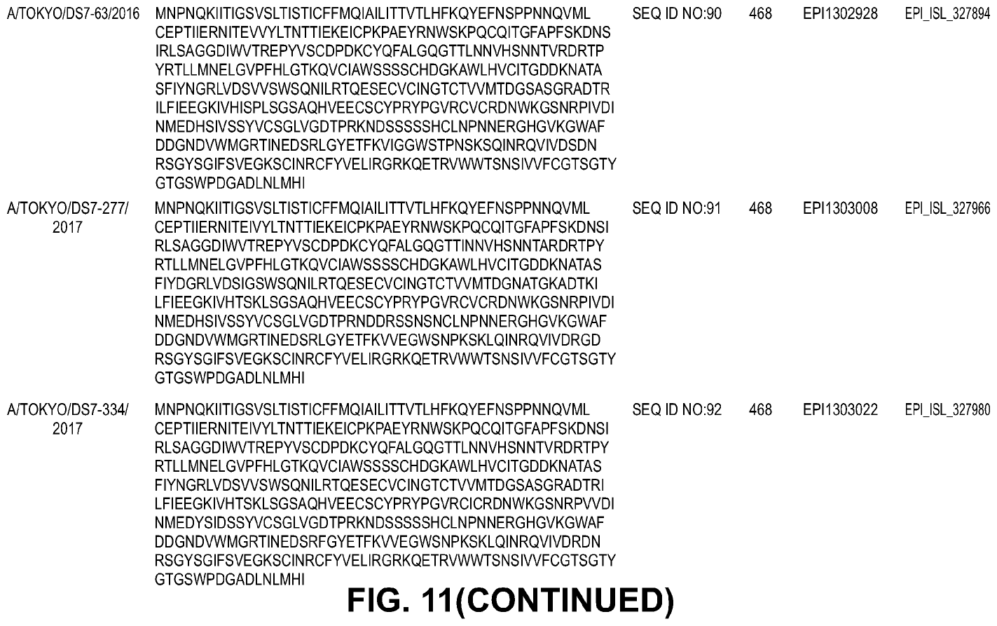

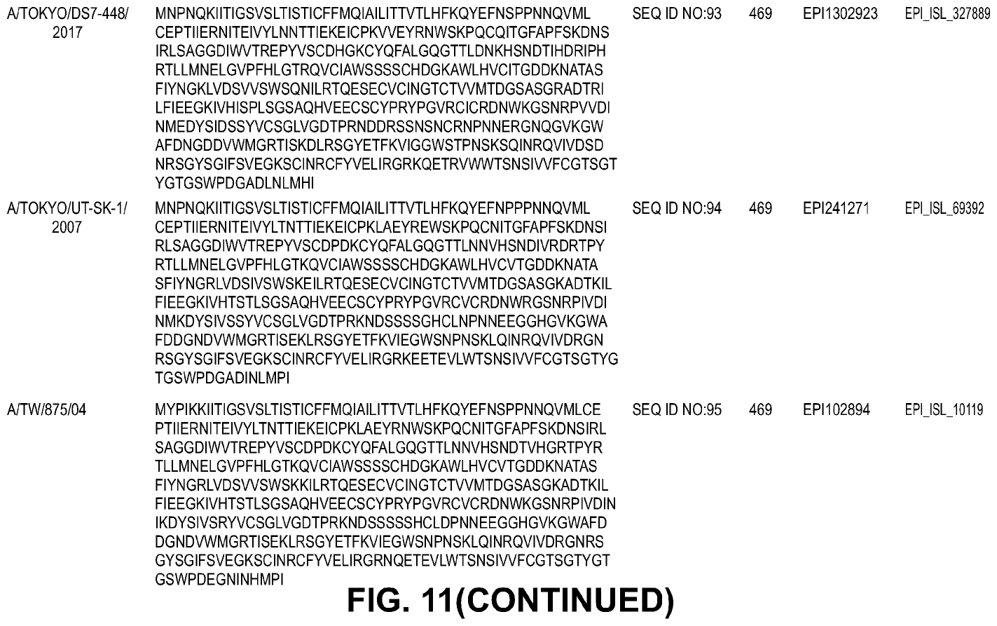

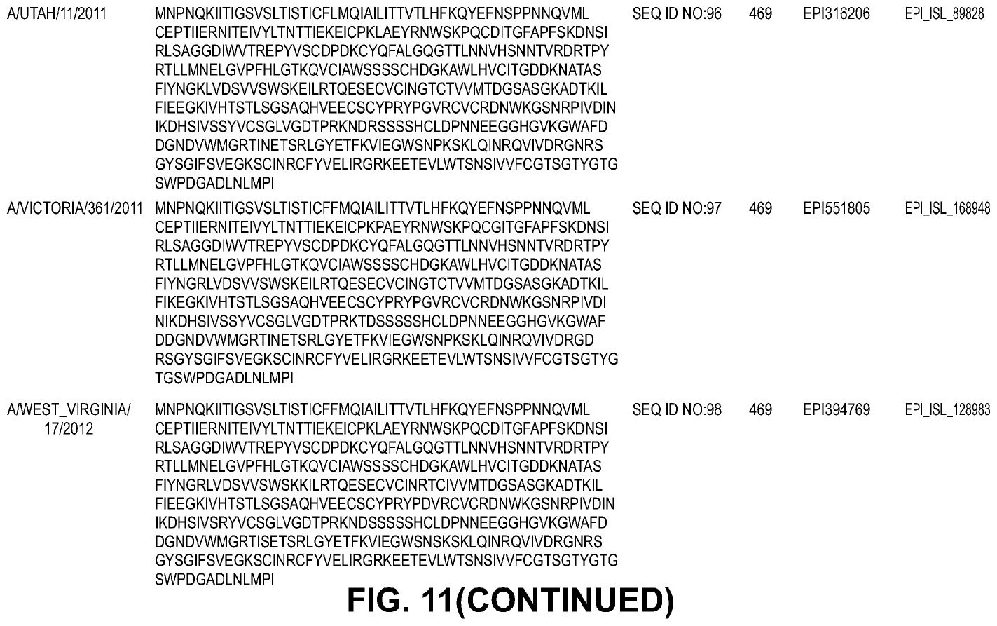

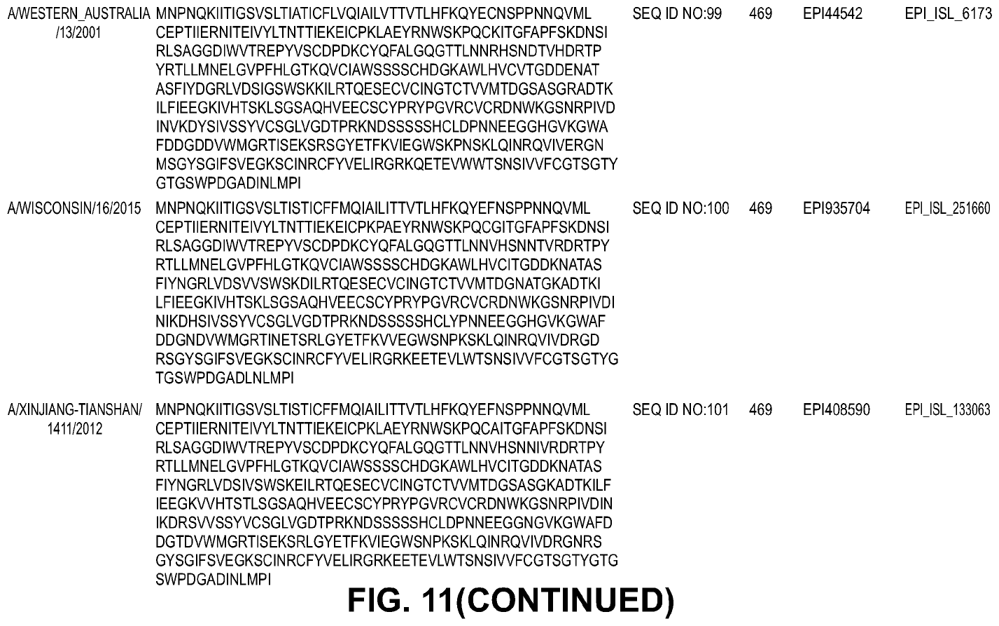

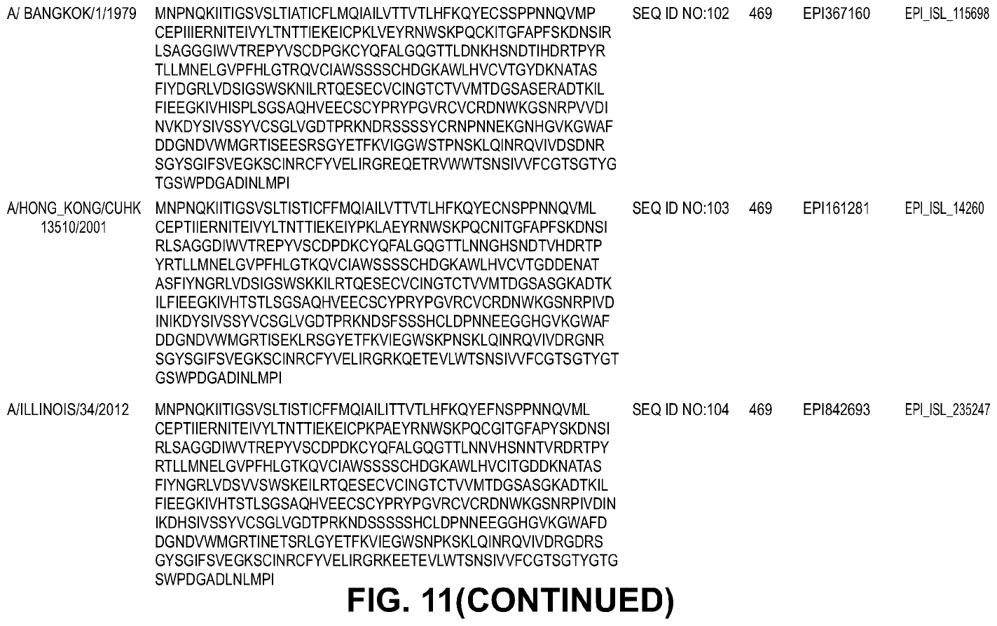

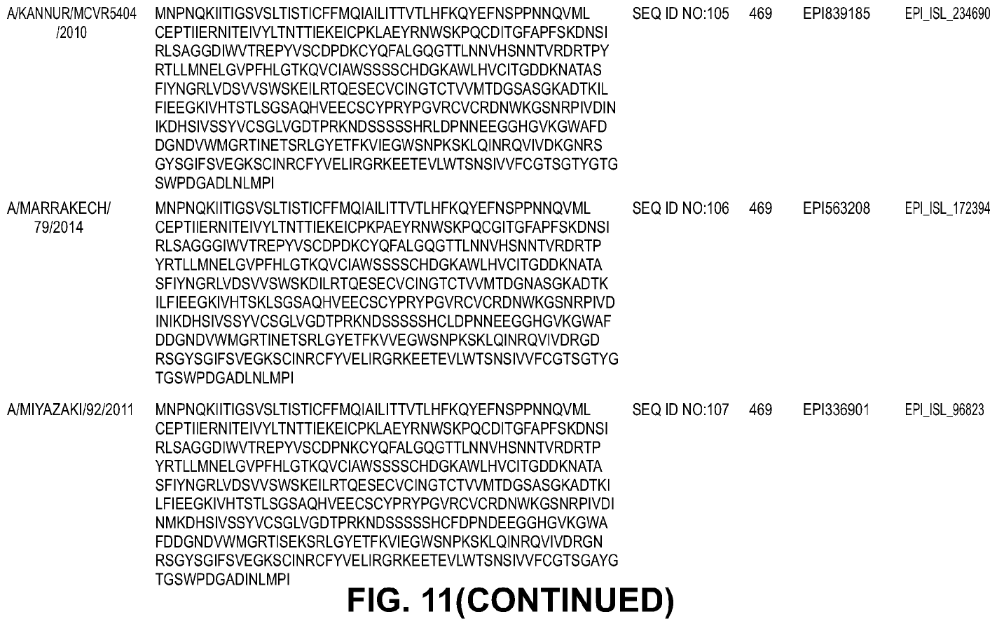

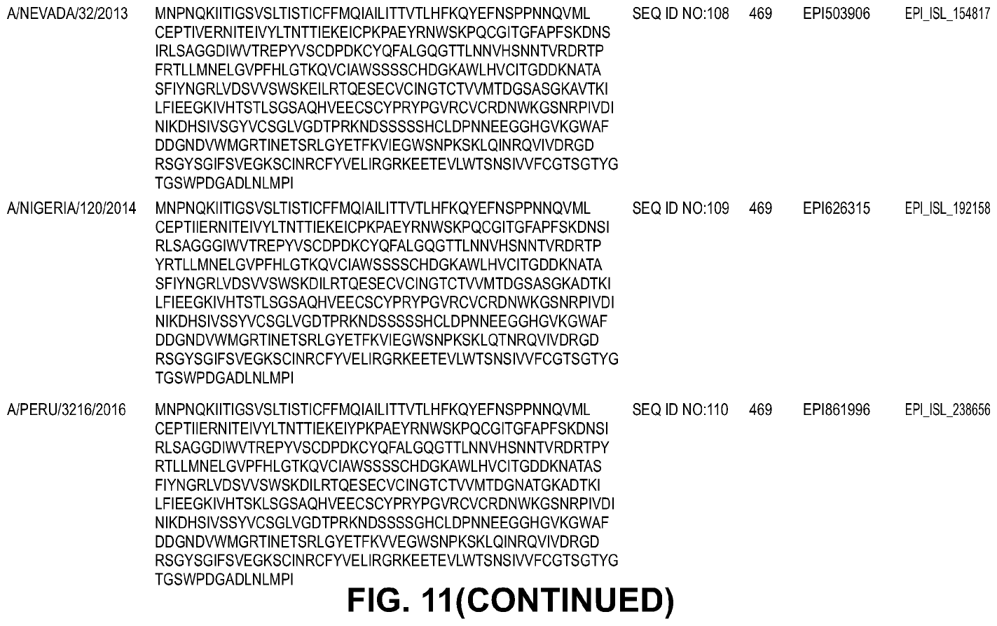

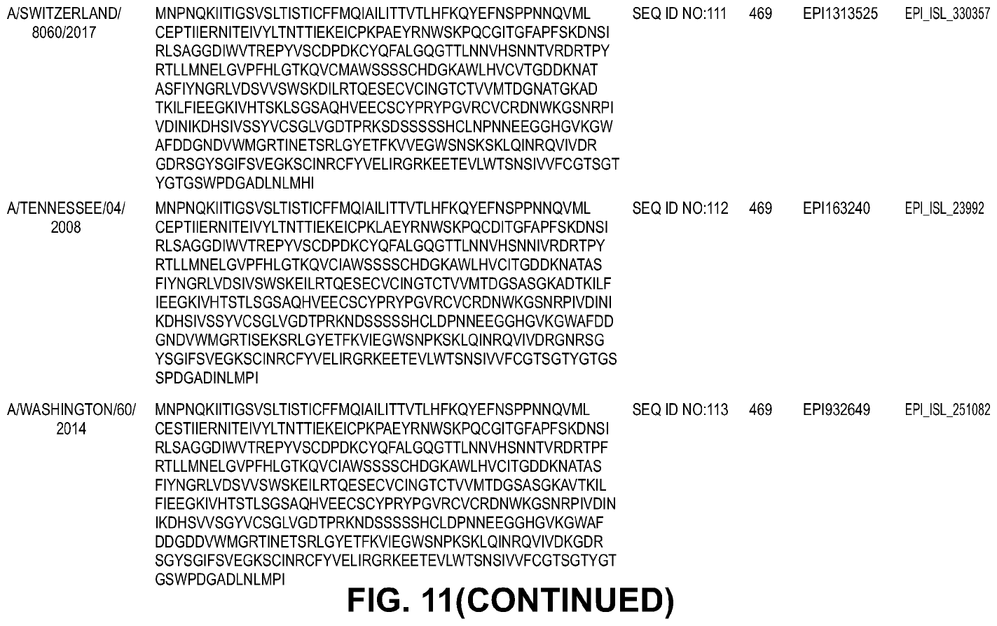

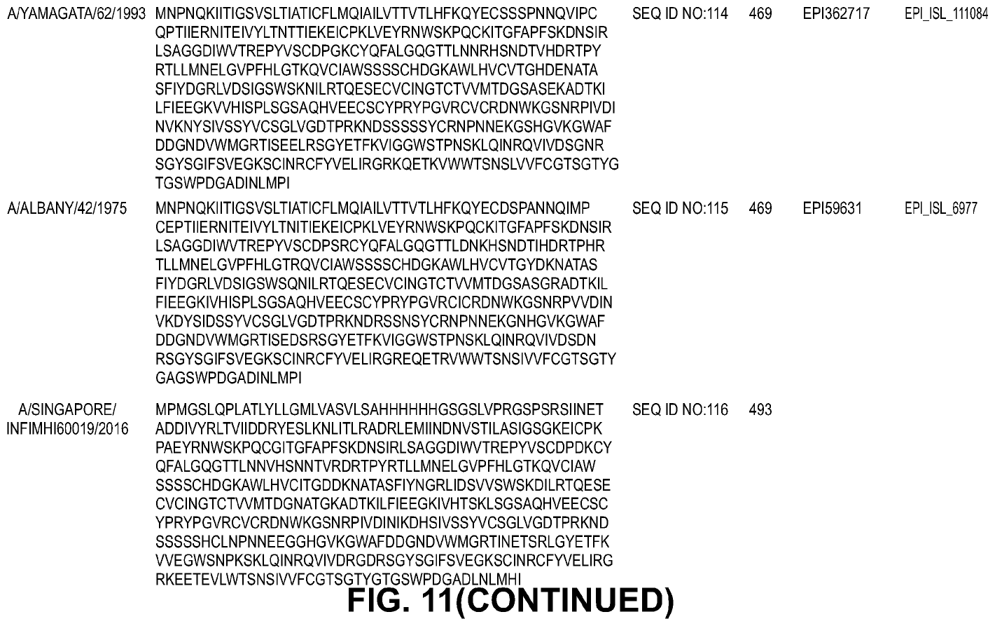

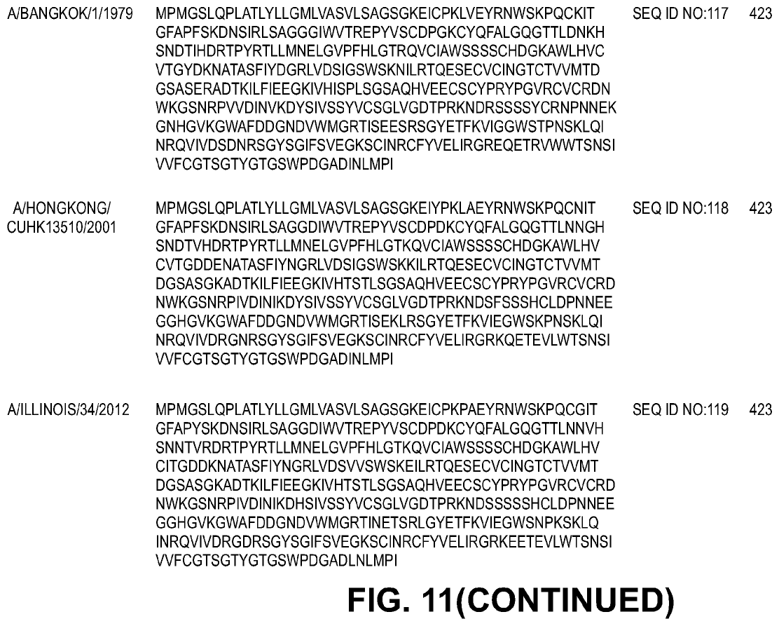

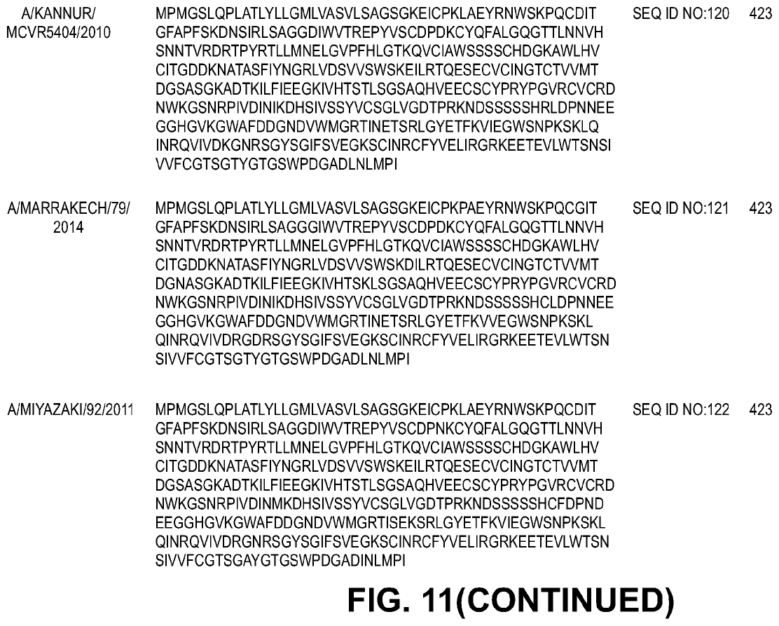

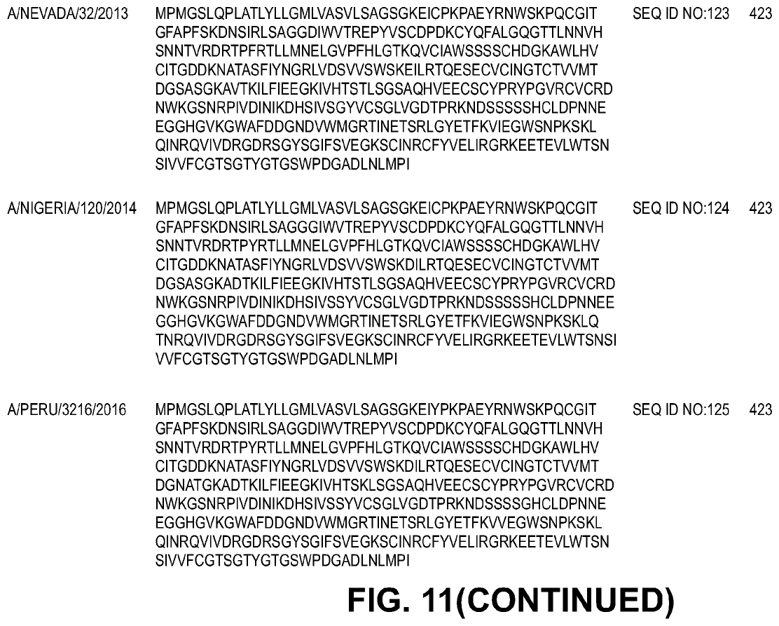

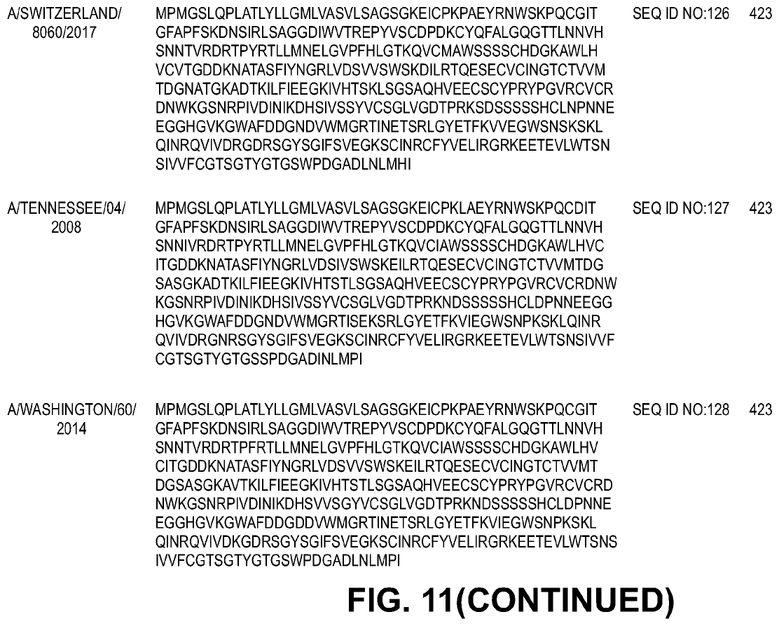

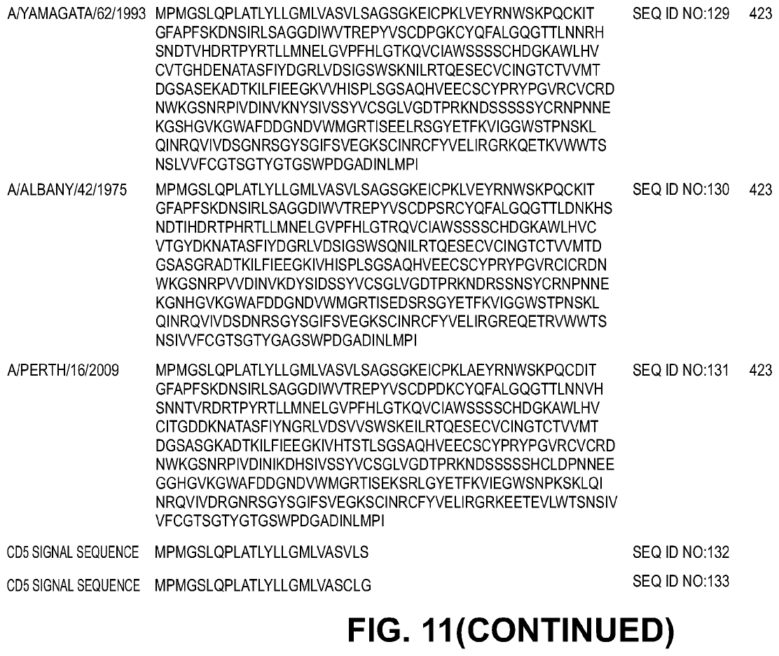

Fitch ferrets were immunized according to the schedule shown in FIG. 10A. Specifically, ferrets were immunized on day 0 with a single intranasal (IN) dose of a H1N1 virus reassortant containing wild type KANS17 NA and on day 21 with a single intramuscular (IM) dose of unadjuvanted, purified KANS17 dTM75 or tet-NA, and animals were bled 21 days later to evaluate NA-specific responses by ELISA and ELLA, as described above.

The KANS17 dTM75 variant N2 efficiently boosted IgG specific-NA responses in pre-immunized ferrets as measured by ELISA, with the dTM75 variant N2 producing slightly higher ELISA titers than the tet-NA control. FIG. 10B. the dTM75 variant from BELG15 and KANS17 produced ELISA titers comparable to the tet-NA control. Single IM vaccination with dTM75-NA designs was able to boost NAI responses in pre-immune ferrets by 3-fold (PERTH09) to 5-fold (BELG15 and KANS17) compared to mock-vaccinated animals (groups that were H1N2 virus pre-immunized only). FIG. 10B.

Overall, these pre-immune ferret data show that the tetrameric NA formed using the N2 truncated stalk design disclosed in this application is highly immunogenic. High NA-specific antibody responses were observed in virus pre-exposed ferrets, even after a single immunization and in the absence of adjuvant.

### Example 8. Immunogenicity and Protection Against Viral Infection and/or Disease Severity in Naïve Ferrets Immunized with a Recombinant, Truncated NA Variant

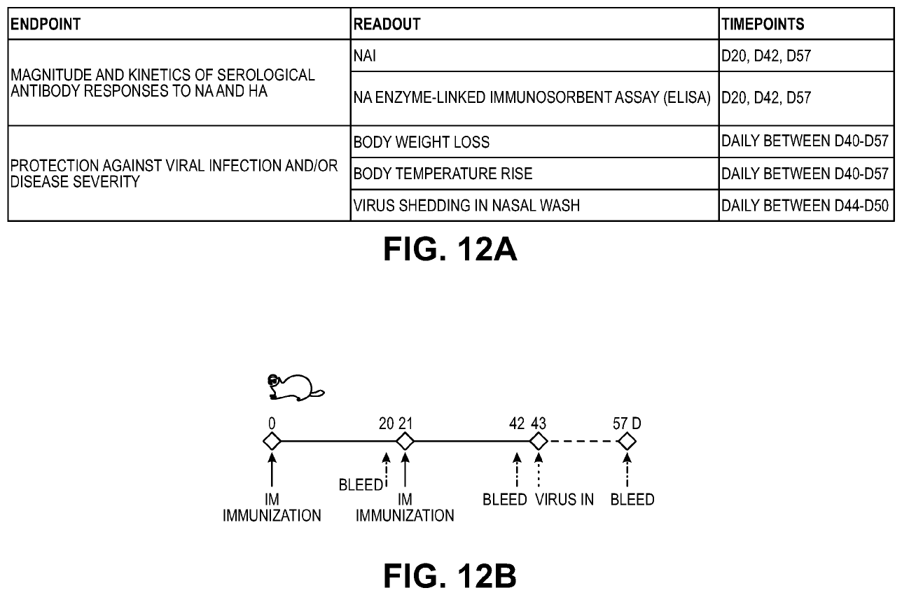

Naïve ferrets immunized with a recombinant, truncated NA variant (PERT09 dTM75) were assessed for immunogenicity, protection against viral infection and/or disease severity. FIG. 12A. For this study, outbred 17-21 week old naïve male Fitch ferrets (n=9/group) were immunized twice intramuscularly (IM) at days 0 and 21 with Ni-affinity purified recombinant PERT09 dTM75 or PERT09 tet-NA as a positive control using a 5 μg dose formulated with AF03 adjuvant or a 45 μg dose with or without adjuvant. Negative control groups were injected IM on days 0 and 21 with phosphate buffered saline (Mock) or Mock+adjuvant. An additional control group was inoculated intranasally on day 0 with a low dose (10e3 Plaque-forming Units (PFU)) of homologous H3N2 virus (A/PE/09 pre-infected), followed by Mock on day 21. A ferret group, immunized with 45 μg dose of Recombinant HA (rHA), was also used as a control. See Table 4.

All groups were bled one day before the second (booster) dose (day 20) and then three weeks later (day 42) before viral challenge. FIG. 12B. To assess immunogenicity, sera samples (stored at −20° C. until required) were assayed for NAI (IC50) using ELLA or NA-specific IgG titer (EC50) using ELISA, as described above in Example 6.

On day 43, all ferret groups were intranasally inoculated with 10⁶ PFU of PERT09 H3N2 wild-type influenza A challenge virus (1,000 μL/dose, split evenly between nostrils). An additional control group received TAMIFLU® (10 mg/kg/day) via the intragastric route, split in two daily doses, with the initial dose starting 6 hours post-challenge. The animals were monitored for 14 days post-challenge for clinical symptoms including body weight changes (once daily) and changes in temperature (twice daily). Sera were collected on day 57 after challenge and stored at −20° C. until ELLA or ELISA testing as described above. FIG. 12B.

To assess total viral shedding, nasal washes were collected from all challenged animals daily between days land 7 post-challenge and samples were stored at or below −65° C. Ferrets were anesthetized with a ketamine (25 mg/kg) and xylazine (2 mg/kg) mixture, and 0.5 mL of sterile PBS containing penicillin (100 U/mL), streptomycin (100 μg/mL), and gentamicin (50 μg/mL) was injected into each nostril, collected and stored at or below −65° C. Virus in the nasal wash specimens was titrated by standard 50% tissue culture infectious dose (TCID₅₀) assay. The nasal washes were thawed and then clarified by centrifugation. The resulting supernatant was serially diluted 10-fold then transferred into respective wells of a 96-well plate which contains a monolayer of Madin-Darby Canine Kidney Cells (MDCK) cells for titration.

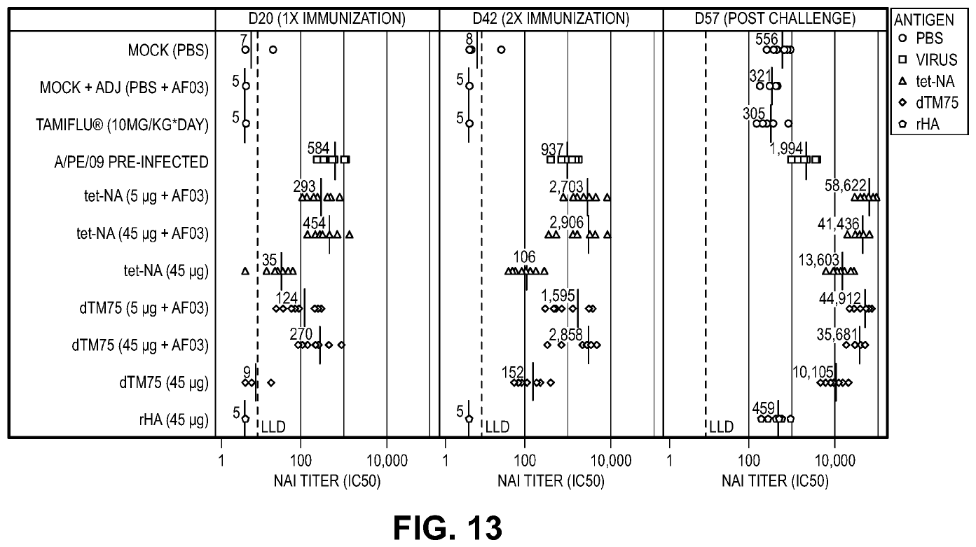

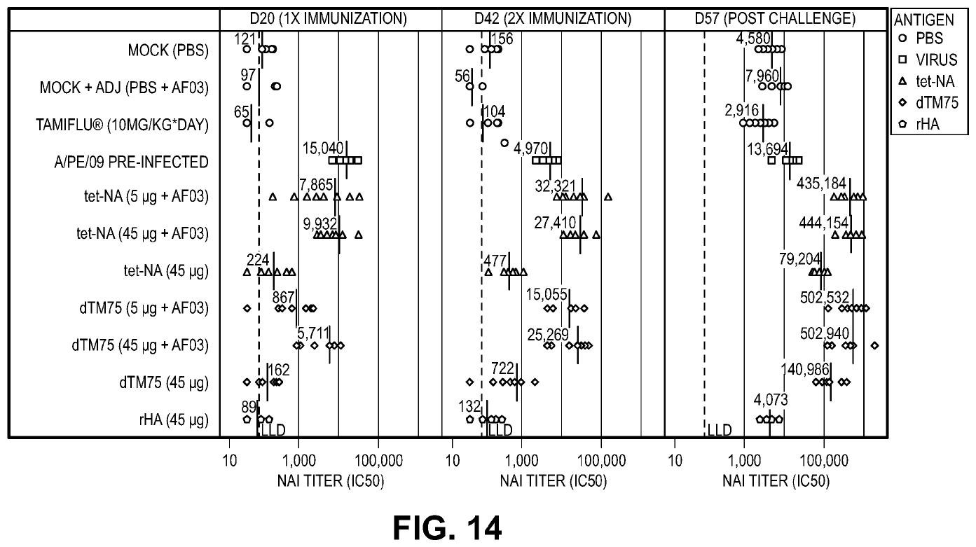

As shown in FIG. 13, dTM75 based on PERT09 elicited higher levels of NAI titers before (day 20 and day 42) and after challenge (day 57) in comparison to Mock (with or without adjuvant). The NAI titers were boosted by adjuvant. dTM75 with adjuvant (5 μg and 45 μg) induced NAI titers after the second immunization, which were at a higher level than those induced by a prior infection. The addition of adjuvant was dose sparing (e.g., 5 μg+AF03 is more immunogenic than 45 μg without adjuvant). Similar trends were observed for NA ELISA titers. FIG. 14.

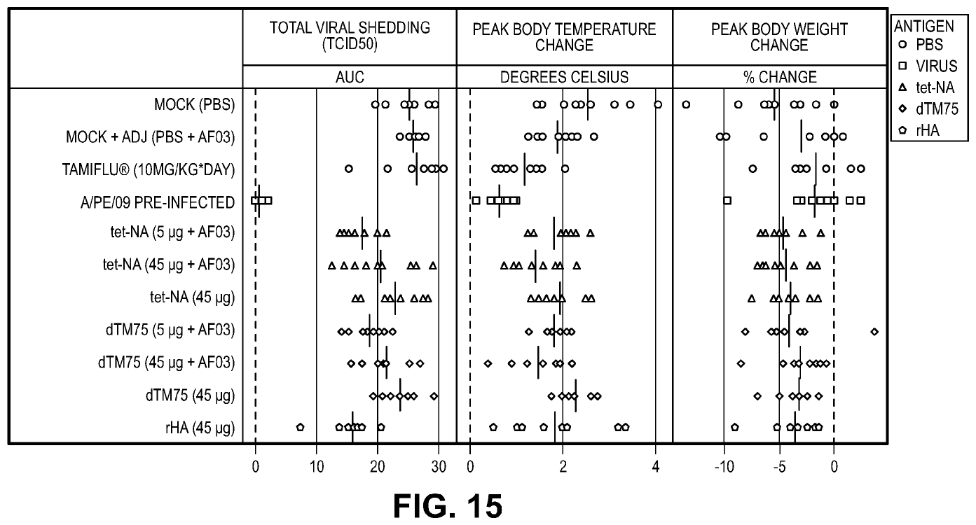

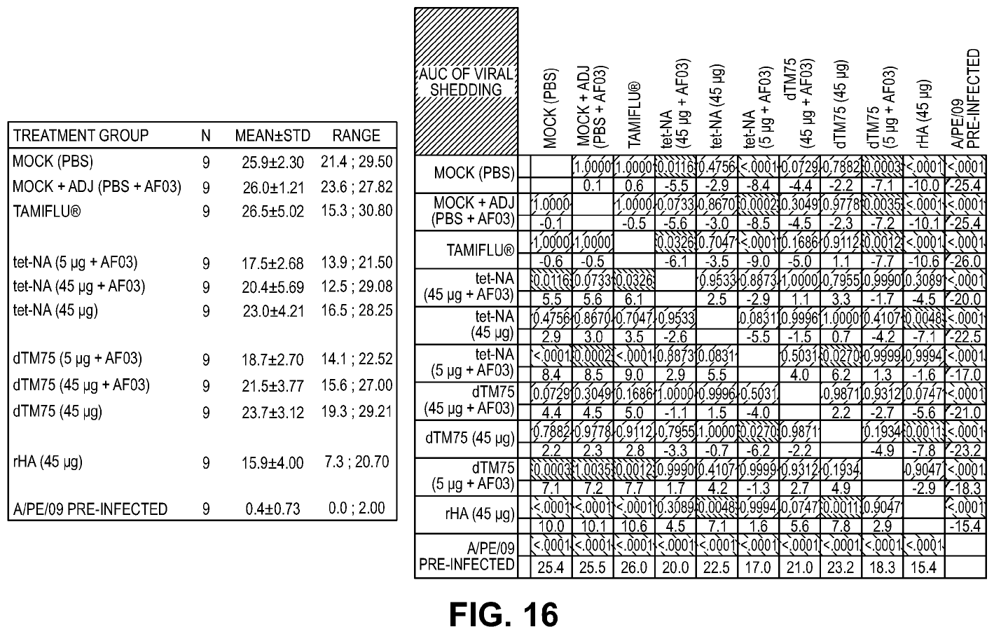

As shown in FIGS. 15-18, PERT09 dTM75 vaccination reduced viral shedding and disease severity as indicated by reduced peak body temperature (fever) change and reduced peak body weight change following homologous H3N2 challenge in naïve ferrets in comparison to Mock. NA-mediated protection for viral shedding reduction was characterized by adjuvant-dependency with the 5 μg dosage in the presence of adjuvant resulting in the highest reductions, comparable to reduction in rHA group and significantly higher to reduction in TAMIFLU® group. FIGS. 15 and 16. Although PERT09 dTM75 does not appear to be as efficient as prior infection for reducing viral shedding, this result is expected since infection provides both anti-NA and anti-HA immunity in addition to T-cell immunity against conserved epitopes.

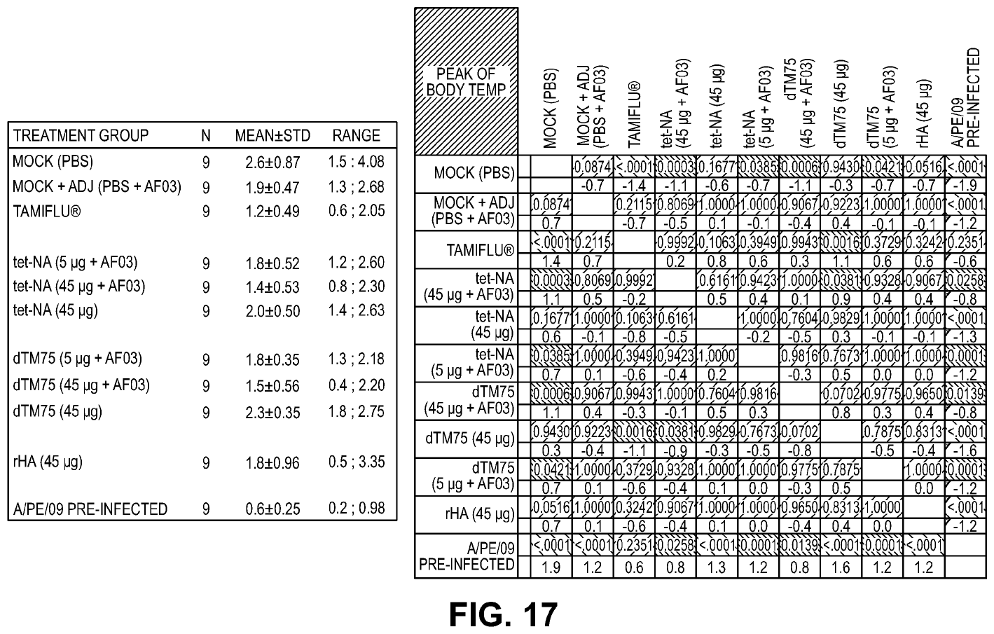

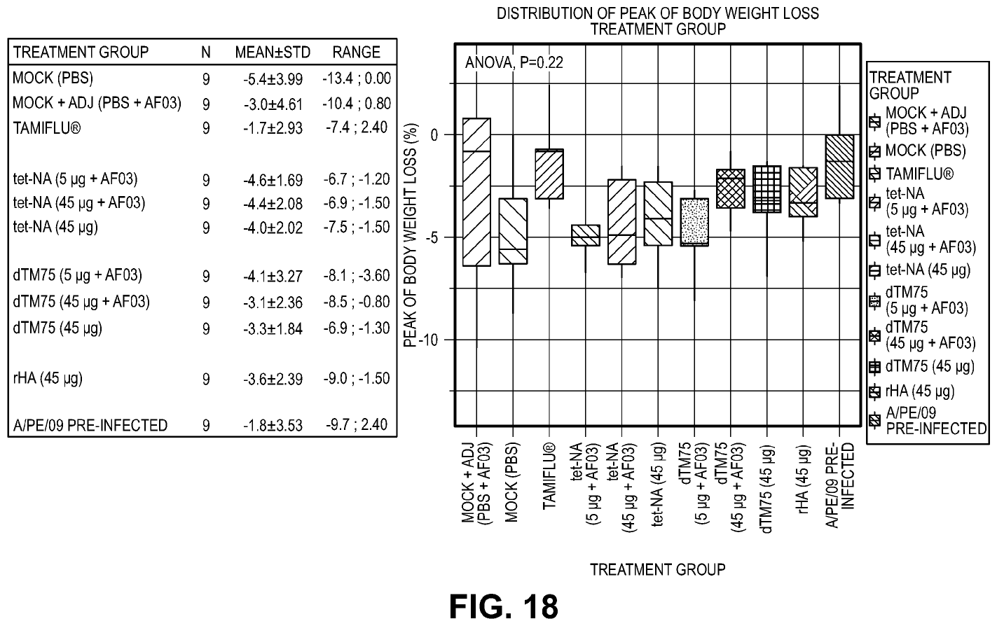

NA-mediated protection against a rise in peak body temperature was characterized by dose- and adjuvant-dependency. Only adjuvanted (5 μg and 45 μg) PERT09 dTM75 or tet-NA doses with AF03 were significantly reduced in comparison to Mock. FIGS. 15 and 17. Although not significant, NA-mediated protection against a reduction in peak body weight was observed across all treatment groups in comparison to Mock. However, these differences were not significant. FIGS. 15 and 18.

Overall, the naïve ferret data show that PERT09 dTM75 induced NA-specific antibody responses, which are associated with protection from infection and disease severity.

Table 4

TABLE 4
Ferret challenge study design to assess immunogenicity and protection by dTM75 NA

| | | | | | | | Challenge | TAMIFLU ® | |
|---|---|---|---|---|---|---|---|---|---|
| | | Prime (D 0)/Boost (D 21) | | | | | (D 43) | Treatment | |
| Group | N | Antigen | Dose (μg) | | Adj. | Route | Virus | (D 43-D 50) | Group name |
| 1 | 9 | Diluent (PBS) | 0 | | AF03 | IM | A/Perth/16/ | None | Mock + adj (PBS + AF03) |
| 2 | 9 | A/Perth/16/2009 H3N2 virus | 10e3 | PFU* | None | IN* | 2009 H3N2 | | A/PE/09 pre-infected |
| 3 | 9 | A/Perth/16/2009 tet-NA | 5 | μg | AF03 | IM | (10e6 PFU) | | tet-NA (5 μg + AF03) |
| 4 | 9 | A/Perth/16/2009 tet-NA | 45 | μg | AF03 | IM | | | tet-NA (45 μg + AF03) |
| 5 | 9 | A/Perth/16/2009 tet-NA | 45 | μg | None | IM | | | tet-NA (45 μg) |
| 6 | 9 | A/Perth/16/2009 dTM75 | 5 | μg | AF03 | IM | | | dTM75 (5 μg + AF03) |
| 7 | 9 | A/Perth/16/2009 dTM75 | 45 | μg | AF03 | IM | | | dTM75 (45 μg + AF03) |
| 8 | 9 | A/Perth/16/2009 dTM75 | 45 | μg | None | IM | | | dTM75 (45 μg) |
| 9 | 9 | Diluent (PBS) | 0 | | None | IM | | | Mock (PBS) |
| 13 | 9 | A/Perth/16/2009 rHA | 45 | μg | None | IM | | | rHA 45 μg |

| | | | | | | | |
|---|---|---|---|---|---|---|---|
| 15 | 9 | Diluent (PBS) | 0 | None | None | 10 mg/kg | TAMIFLU ® (10 |
| | | | | | | daily** | mg/kg*day) |

*Group 2 was inoculated intranasally with low dose of 10e3 PFU of A/Perth/16/2009 H3N2 virus on day 0 and received diluent only (PBS) IM on day 21.
**The TAMIFLU ® dose was administered via the intragastric route, split into two daily doses, the first dose starting 6 hours post-challenge.

### Example 9. Immunogenicity and Protection Against Viral Infection and/or Disease Severity in Naïve Ferrets Immunized with Recombinant Neuraminidase in the Context of a Mismatched Hemagglutinin

A naïve ferret challenge model was used to demonstrate the benefit of a vaccine strategy that combines a recombinant NA with an HA from a mismatched influenza strain. The general vaccination timeline is outlined in FIG. 12B while additional details are summarized in Table 5.

TABLE 5

| | | Prime (D 0)/boost (D 21)/PERT09 challenge (D 43) | | | | |
|---|---|---|---|---|---|---|
| Gr. # | N | Vaccine NA | Vaccine HA | Dose | | Adjuvant |
| 1 | 9 | Diluent (PBS) | | — | | — |
| 2 | 9 | PERT09 virus pre-infected | | 10e3 | PFU | — |
| 6 | 9 | PERT09 | | 5 | ug | AF03 |
| | | dTM75-NA | | | | |
| 7 | 9 | | SING16-rHA | 5 | ug | AF03 |
| 8 | 9 | PERT09 | SING16-rHA | 5/5 | ug | AF03 |
| | | dTM75-NA | | | | |
| 3 | 9 | PERT09 | | 45 | ug | — |
| | | dTM75-NA | | | | |
| 4 | 9 | | SING16-rHA | 45 | ug | — |
| 5 | 9 | PERT09 | SING16-rHA | 45/45 | ug | — |

dTM75-NA

For this study, outbred 17-21 week old naïve male Fitch ferrets (n=9/group) were immunized twice intramuscularly (IM; 500 μL/dose) at days 0 and 21 with 5 μg or 45 μg of either 1) A/Perth/16/2009 recombinant N2 dTM75, 2) a mismatched A/Singapore/INFIMH160019/2016 recombinant hemagglutinin (rHA), or 3) a combination of both PERT09 dTM75-NA and SING16 rHA. 5 μg dose was administered with AF03 adjuvant at a 1:1 ratio. Three weeks after booster vaccination, the ferrets received intranasal challenge with 10′PFU of A/Perth/16/2009 H3N2 wild-type influenza A virus (1,000 μL/dose, split evenly between nostrils). The animals were monitored for 14 days post-challenge for clinical symptoms and changes in body weight once daily, and body temperature twice daily. Nasal washes were collected from all challenged animals every day 1-7 post-challenge and samples were stored at ≤−65° C. for viral shedding assessment. Group 1 was immunized with PBS diluent only and served as a negative control to demonstrate the most severe symptoms after the challenge/infection. Group 2 received live intranasal challenge with A/Perth/16/2009 H3N2 virus (pre-infected) on DO instead of vaccination with PERT09 dTM75-NA or SING16-rHA, and served as a positive control as it was the best protected from the challenge. Endpoints of the study were assessed as summarized in Table 6.

TABLE 6

| Endpoint | Assay | Specificity | Timepoints |
|---|---|---|---|
| Serology | ELLA | PERT09 | D 20, D 42, D 57 |
| | NA ELISA | PERT09 | |
| | HAI | PERT09, | |
| | | SING16 | |
| Protection | Weight loss, temperature | N/A | Daily post-challenge, |
| | | | between D 43-D 57 |
| | | | (for 14 days) |
| | TCID50 nasal wash | N/A | Daily post-challenge, |
| | | | between D 43-D 50 |
| | | | (for 7 days) |

All ferrets were bled under sedation at baseline (DO) and three weeks after primer (one day before or just before booster, D20) and booster vaccination (D42), and two weeks after challenge (D57), and sera samples (stored at −20° C. until required) were tested by ELLA, as described above, to assess NAI activity, by NA ELISA, as described above, for binding antibody levels and by the hemagglutinin inhibition assay (HAI) to assess antibody responses to hemagglutinin antigens of either A/Perth/16/2009 virus or SING16-rHA. The animals were monitored for 14 days post-challenge for clinical symptoms and changes in body weight once daily, and body temperature twice daily (AM and PM). Nasal wash specimens were collected from experimentally infected ferrets daily following intranasal challenge. Virus in the nasal wash specimens was titrated by standard 50% tissue culture infectious dose (TCID50) assay as follows. The nasal washes were thawed and then clarified by centrifugation. The resulting supernatant was serially diluted 10-fold then transferred into respective wells of a 96-well plate for titration on a monolayer of Madin-Darby Canine Kidney Cells (MDCK) cells.

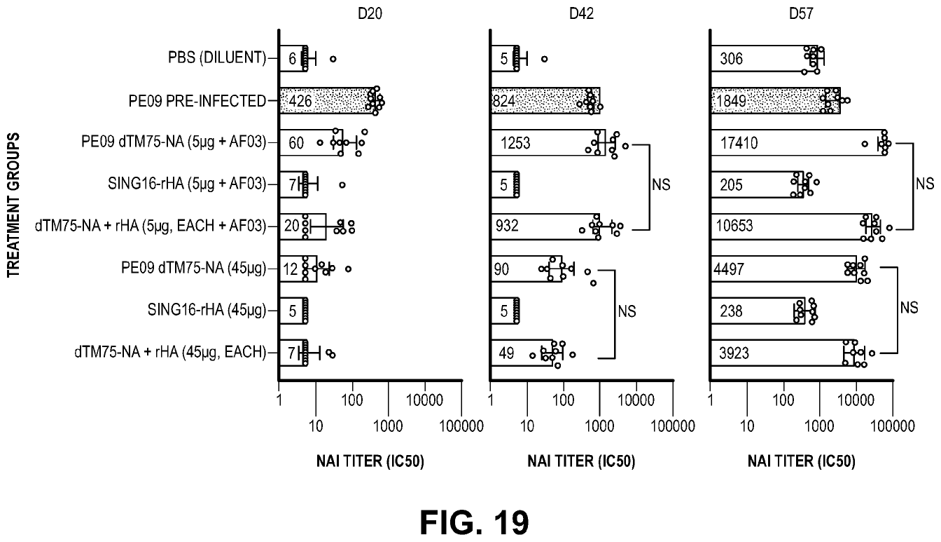

NAI antibody responses were measured against H6 reassortant viruses containing NA derived from strains of interest by ELLA substantially as described above in Example 6. Sera were collected three weeks after initial dose or prime and three weeks after booster vaccination and tested via ELLA to assess NAI antibody activity using H6N2 A/Perth/16/2009 viruses as sources of sialidase, respectively. Comparison of NAI responses between the groups immunized with a single PERT09 dTM75-NA or the combination of dTM75NA and rHA did not reveal any statistically significant differences. FIG. 19. Robust induction of anti-NA A/Perth/16/2009 titers (ELLA) over-time (D20, D42 and D57) was observed in all ferrets administered either live virus (PERT09 pre-infected) or immunized with low dose (5 μg+AF03) or high dose (45 μg) PERT09 dTM75-NA. FIG. 19. Vaccine formulations containing low dose (5 μg+AF03) dTM75-NA (dTM75-NA alone or dTM75-NA+rHA) induced higher NAI titers than vaccine formulations containing high dose (45 μg) dTM75-NA. FIG. 19.

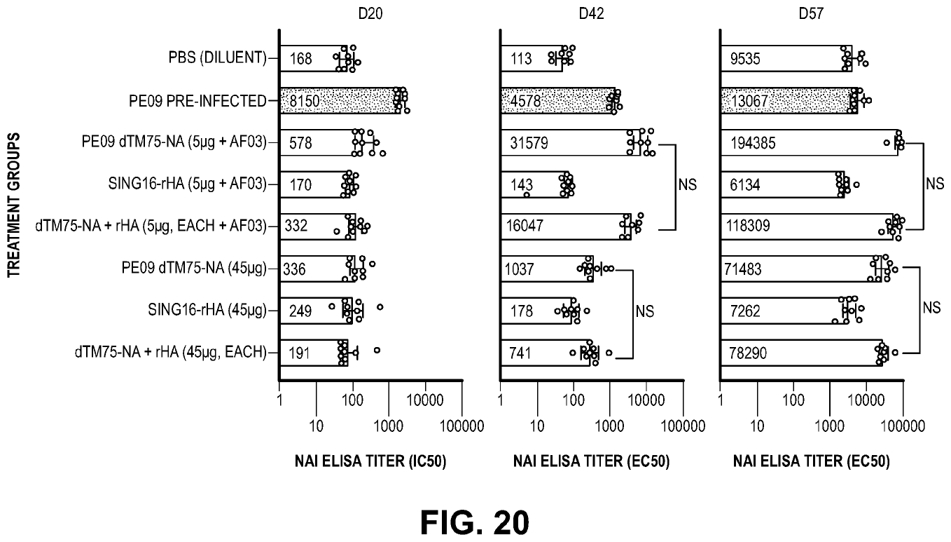

Robust induction of anti-NA A/Perth/16/2009 binding (ELISA) titers over-time (D20, D42 and D57) was observed in all ferrets administered either live virus (PERT09 pre-infected) or immunized with low dose (5 μg+AF03) or high dose (45 μg) PERT09 dTM75-NA. FIG. 20. Sera were collected three weeks after initial dose or prime and three weeks after booster vaccination and tested via ELISA to assess NA-binding titers, substantially as described above in Example 6. Comparison of ELISA NA-binding titers between the groups immunized with a single PERT09 dTM75-NA or the combination of dTM75-NA and rHA did not reveal any statistically significant differences in agreement with ELLA data. As with NAI titers, vaccine formulations containing low dose (5 μg+AF03) dTM75-NA (dTM75-NA alone or dTM75-NA+rHA) induced higher ELISA titers than vaccine formulations containing high dose (45 μg) dTM75-NA. FIG. 20.

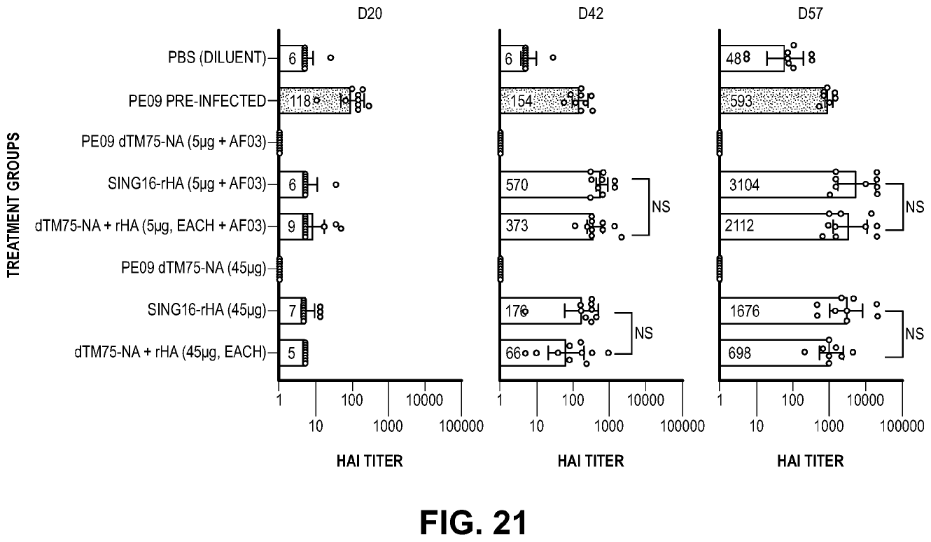

Robust SING16 HAI antibody titers were induced after the second immunization (D42) by vaccine formulations containing either rHA or dTM75-NA+rHA without an additional boost. FIG. 21. Sera were collected three weeks after initial dose or prime and three weeks after booster vaccination and tested via HAI to assess responses to A/Singapore/INFIMH160019/2016 rHA. Comparison of HAI titers between the groups immunized with a single SING16-rHA or the combination of dTM75NA and rHA did not reveal any statistically significant differences in agreement with ELLA and ELISA data. FIG. 21. Interestingly, an additional SING16 HAI titer boost (4-10 times) was observed on D57 after the challenge with A/Perth/16/2009 virus, indicating a cross-reactive HAI titer increase after a heterologous challenge. FIG. 21. Consistent with NAI and ELLA titers, vaccine formulations containing low dose (5 μg+AF03) dTM75-NA (dTM75-NA alone or dTM75-NA+rHA) induced higher SING16 HAI titers than vaccine formulations containing high dose (45 μg) dTM75-NA. FIG. 21.

To measure HAI titers, sera were treated with receptor-destroying enzyme (RDE; Denka Seiken, Co., Japan) to inactivate nonspecific inhibitors prior to HAI assay. Briefly, three parts RDE were added to one part serum and incubated overnight at 37° C. RDE was inactivated by incubation at 56° C. for 30 min with 6 times the serum volume and added to 0.9% saline RDE-treated serum 2-fold serially diluted in v-bottom 96-well microtiter plates. An equal volume of each virus from the panel, adjusted to 4 hemagglutinating units (HAU)/50 μL, was added to each well. For the current study, homologous virus panel included A/Singapore/INFIMH-16-0019/2016 (H3N2) virus grown in eggs. The plates were covered and incubated at room temperature for 20 minutes, followed by the addition of 1% chicken erythrocytes (CRBC) (Lampire Biologicals) in PBS. The plates were mixed by agitation and covered, and the RBCs were allowed to settle for 1 hour at room temperature. The HAI titer was determined by the reciprocal dilution of the last well which contained non-agglutinated RBCs.

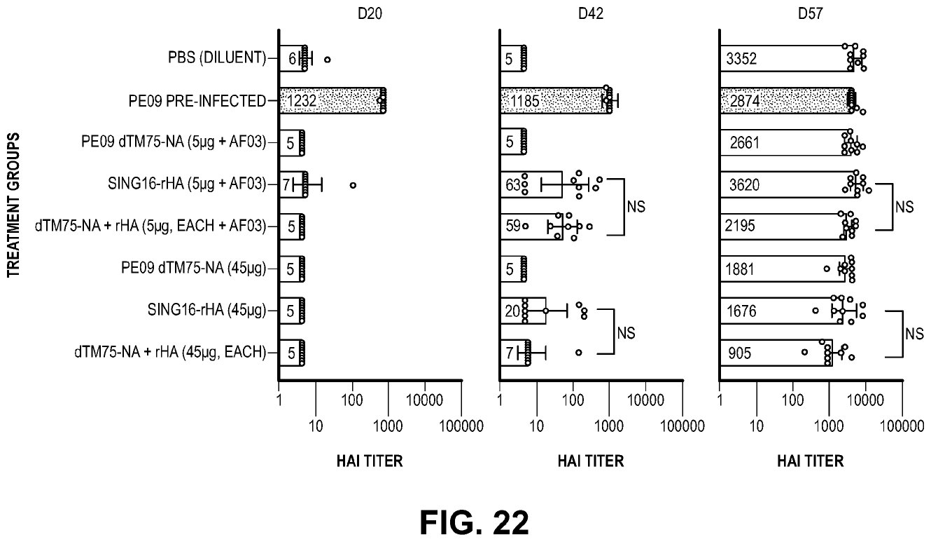

Robust PERT09 HAI antibody homologous titers were induced after A/Perth/16/2009 virus challenge (D57) by vaccine formulations containing either rHA or dTM75-NA+rHA without an additional boost. FIG. 22. Sera were collected three weeks after initial dose or prime and three weeks after booster vaccination and tested via HAI to assess antibody titers capable of neutralizing A/Perth/16/2009 virus. Comparison of HAI titers between the groups immunized with a single SING16-rHA or the combination of dTM75-NA and SING16-rHA did not reveal any statistically significant differences in agreement with the ELLA and ELISA data. FIG. 22. Interestingly, a low level of cross-reactivity was observed on day 42 in groups immunized with SING16-rHA or the combination of SING16-rHA and dTM75-NA, which demonstrated detectable heterologous titers (>50 HAI titer) against PERT09 virus. FIG. 22.

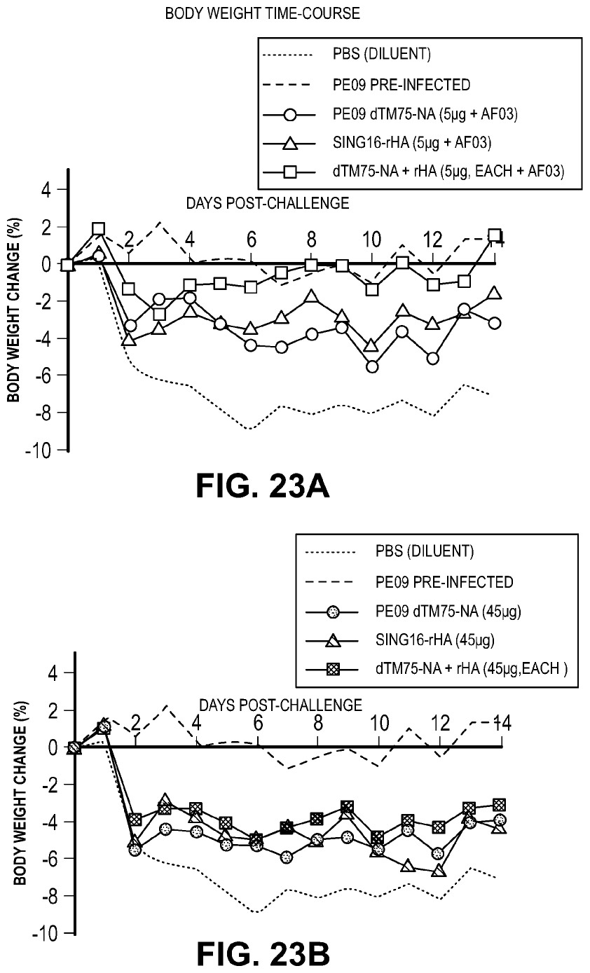

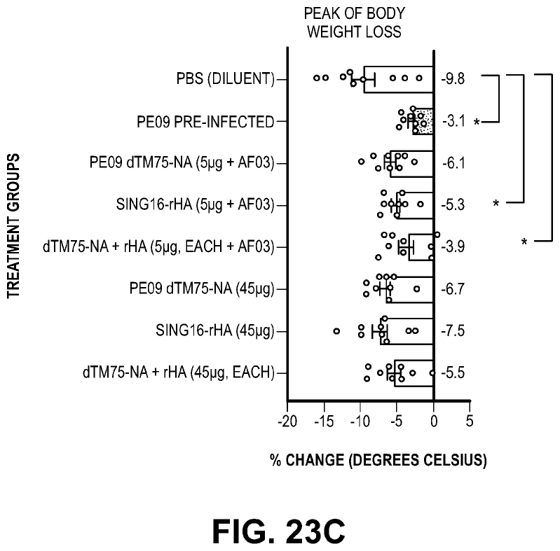

Immunization with two doses of either PERT09 dTM75-NA or SING16 rHA, administered individually or in combination, provided protection against body weight loss induced by challenge with live A/Perth/16/2009 virus. FIGS. 23A-C. Changes in body weight were observed for the period of 14 days post-challenge (FIG. 23A-B). Unvaccinated animals (PBS control group) lost up to 9.8% of body weight by day 6, while 5 μg SING16 rHA+AF03 or the combination of 5 μg SING16 rHA+5 μg PERT09 dTM75-NA+AF03 lost 5.3% and 3.9%, respectively (FIG. 23C). Vaccinated animals seemed to recover body weight faster than PBS unvaccinated group. A vaccine formulation comprising 5 μg dTM75NA in combination with 5 μg rHA and AF03 provides a protection from a weight loss similarly to PERT09-preinfected ferrets. FIGS. 23 A and C.

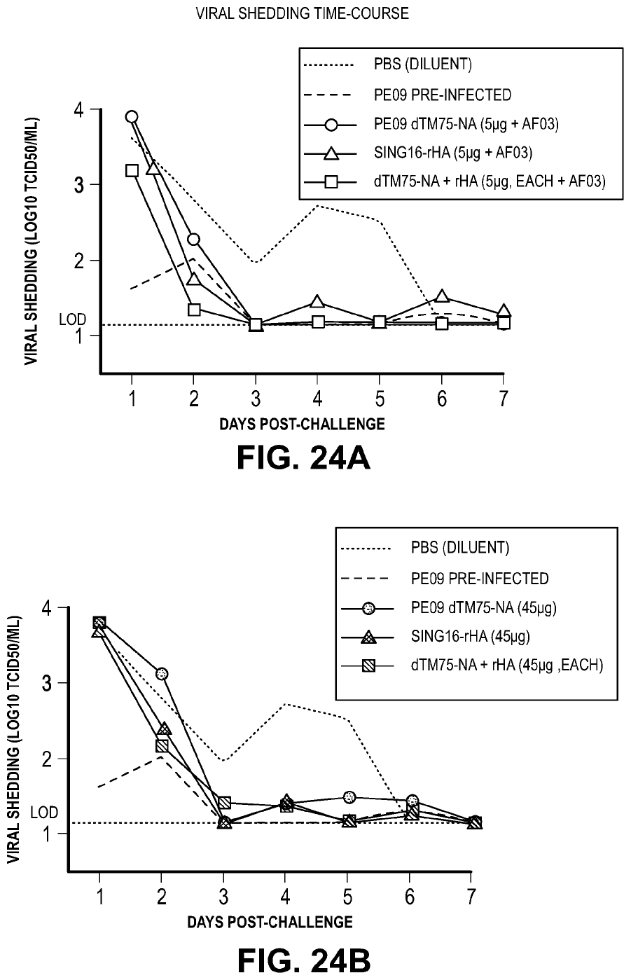

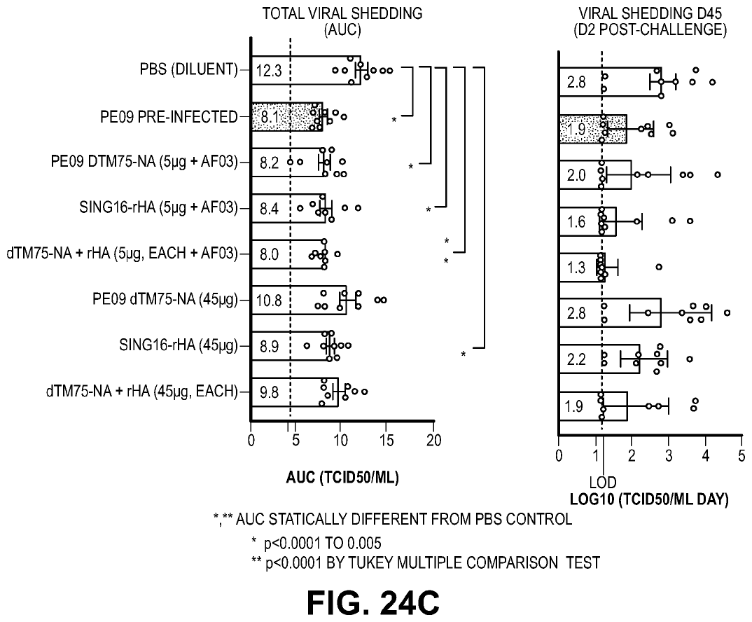

Viral shedding was assessed daily for 7 days post-challenge and changes in TCID50/ml titers are presented in FIGS. 24A-B. The duration and intensity of viral shedding (AUC) are particularly reduced for the vaccine formulation comprising 5 μg dTM75NA in combination with 5 μg rHA and AF03. FIGS. 24A-C. AUC was calculated for all groups and a pairwise comparison analysis demonstrated that all low dose (5 μg+AF03) groups (dTM75-NA, rHA, or the combination of dTM75-NA and rHA) induced a statistically significant reduction in viral shedding in comparison to PBS-control group (FIG. 24C). Additionally, log 10 TCID50/ml values 2 days post-challenge demonstrated the lowest viral shedding titers in the low dose (5 μg+AF03) combination group (1.3 Log 10) and rHA alone group (1.6 Log 10), however, these reduced titers were not statistically significantly different from unprotected PBS control group (2.8 Log 10).

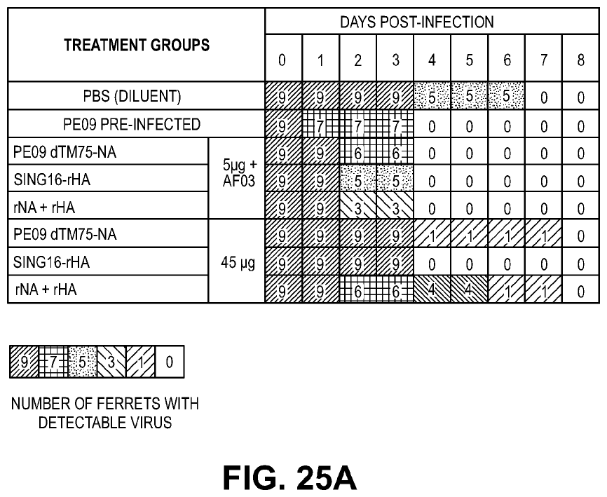

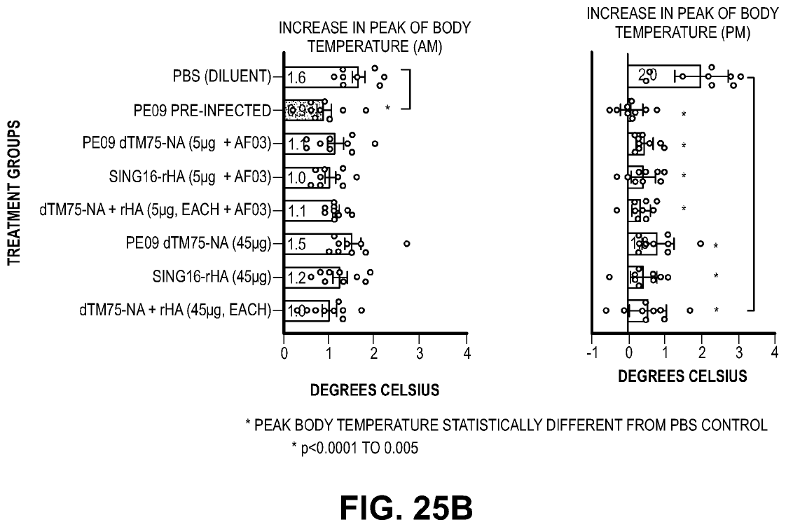

In all groups vaccinated with the lower dose (5 μg+AF03), the duration of infection and number of infected ferrets over time was reduced from 6 to 3 days in comparison to unvaccinated (PBS) animals (FIG. 25A). The high dose (45 μg) groups demonstrated a reduction in viral shedding duration only in rHA group and unreduced length of infection in both groups vaccinated with high dose dTM75-NA or high dose dTM75-NA+rHA (up to 6 days). FIG. 25A. Body temperatures (BT), monitored daily over 14 day period, demonstrated a fever reduction presented as an increase in peak of body temperature in all vaccinated groups in the afternoon (PM) in comparison to PBS controls. FIG. 25B. For the AM body temperature peak, statistically significant differences were identified only between PBS control and PERT09 pre-infected ferret groups, with an overall peak in body temperatures of 1.6 and 0.9 degrees Celsius, respectively. FIG. 25B.

In summary, in the ferret challenge study, the serological data for the vaccine formulations comprising a combination of the rNA variant and the rHA were similar to the serological data for the vaccine formulations containing either the rNA variant or rHA alone, with the low dose formulations (5 μg+adjuvant), consistently inducing higher titers than the high dose formulations (45 μg with no adjuvant). Notwithstanding the similar serological data, the addition of a recombinant NA variant with a recombinant HA from a mismatched influenza strain provides increased protection in the ferret challenge model. The body weight loss for animals vaccinated with the low dose (5 μg+AF03) combination of dTM75-NA and rHA was significantly different from unvaccinated animals. In addition, animals vaccinated with low dose (5 μg+AF03) rHA or the low dose (5 μg+AF03) combination of dTM75-NA and rHA exhibited significant reduction of viral shedding (AUC) and length of viral clearance.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the protein constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

We claim:

1. A modified monomeric influenza virus subtype 2 neuraminidase, wherein the modified monomeric influenza virus subtype 2 neuraminidase comprises:
a signal peptide; and
a head region of an influenza virus subtype 2 neuraminidase,
wherein the cytoplasmic tail, transmembrane region and all or substantially all of the stalk region of the influenza virus subtype 2 neuraminidase have been replaced by the signal peptide,
wherein the modified monomeric influenza virus subtype 2 neuraminidase does not include a heterologous oligomerization domain, and
wherein expression of the modified monomeric influenza virus subtype 2 neuraminidase in a host cell results in secretion of a tetrameric neuraminidase from the host cell.

2. An artificial nucleic acid encoding the modified monomeric influenza virus subtype 2 neuraminidase of claim 1.

3. A tetrameric neuraminidase, comprising four copies of a modified monomeric influenza virus subtype 2 neuraminidase, wherein the modified monomeric influenza virus subtype 2 neuraminidase comprises:
a head region of an influenza virus subtype 2 neuraminidase,
wherein the modified monomeric influenza virus subtype 2 neuraminidase does not contain a cytoplasmic tail, a transmembrane region and all or substantially all of a stalk region of the influenza virus subtype 2 neuraminidase, and
wherein the modified monomeric influenza virus subtype 2 neuraminidase does not include a heterologous oligomerization domain.

4. The modified monomeric influenza virus subtype 2 neuraminidase of claim 1, the artificial nucleic acid molecule of claim 2, or tetrameric neuraminidase of claim 3, wherein following expression of the modified monomeric influenza virus subtype 2 neuraminidase in the host cell, a host cell supernatant comprises modified influenza virus subtype 2 neuraminidase comprising at least the tetrameric neuraminidase and a monomeric form of the modified monomeric influenza virus subtype 2 neuraminidase, and wherein the tetrameric neuraminidase represents at least 40%, at least 60%, at least 80%, or at least 90% of the modified influenza virus subtype 2 neuraminidase in the host cell supernatant, as measured by size exclusion chromatography.

5. The modified monomeric influenza virus subtype 2 neuraminidase or the artificial nucleic acid molecule of any one of claims 1, 2, or 4, wherein amino acid 1 to at least amino acid 70-82 of an influenza virus subtype 2 neuraminidase have been replaced by the signal peptide; or the tetrameric neuraminidase of claim 3 or 4, wherein the modified monomeric influenza virus subtype 2 neuraminidase lacks the stalk region of the influenza virus subtype 2 neuraminidase or lacks amino acids 1 through 70-82 of the influenza virus subtype 2 neuraminidase.

6. The modified monomeric influenza virus subtype 2 neuraminidase or the artificial nucleic acid molecule of claim 5, wherein amino acids 1-70, 1-71, 1-72, or 1-73 of the influenza virus subtype 2 neuraminidase have been replaced by the signal peptide in the modified monomeric influenza virus subtype 2 neuraminidase; or the tetrameric neuraminidase of claim 5, wherein the modified monomeric influenza virus subtype 2 lacks amino acids 1-70, 1-71, 1-72, or 1-73 of the influenza virus subtype 2 neuraminidase.

7. The modified monomeric influenza virus subtype 2 neuraminidase or the artificial nucleic acid molecule of claim 5, wherein amino acids 1-74 of the influenza virus subtype 2 neuraminidase have been replaced by the signal peptide in the modified monomeric influenza virus subtype 2 neuraminidase; or the tetrameric neuraminidase of claim 5, wherein the modified monomeric influenza virus subtype 2 neuraminidase lacks amino acids 1-74 of the influenza virus subtype 2 neuraminidase.

8. The modified monomeric influenza virus subtype 2 neuraminidase or the artificial nucleic acid molecule of claim 5, wherein amino acids 1-75, 1-76, 1-77, 1-78, or 1-79 of the influenza virus subtype 2 neuraminidase have been replaced by the signal peptide in the modified monomeric influenza virus subtype 2 neuraminidase; or the tetrameric neuraminidase of claim 5, wherein the modified monomeric influenza virus subtype 2 neuraminidase lacks amino acids 1-75, 1-76, 1-77, 1-78, or 1-79 of the influenza virus subtype 2 neuraminidase.

9. The modified monomeric influenza virus subtype 2 neuraminidase or the artificial nucleic acid molecule of claim 5, wherein amino acids 1-80, 1-81, or 1-82 of the influenza virus subtype 2 neuraminidase have been replaced by the signal peptide in the modified monomeric influenza virus subtype 2 neuraminidase; or the tetrameric neuraminidase of claim 5, wherein the modified monomeric influenza virus subtype 2 neuraminidase lacks amino acids 1-80, 1-81, or 1-82 of the influenza virus subtype 2 neuraminidase.

10. The modified monomeric influenza virus subtype 2 neuraminidase or the artificial nucleic acid molecule of claim 5, wherein amino acids 1-82 of the influenza virus subtype 2 neuraminidase have been replaced by the signal peptide in the modified monomeric influenza virus subtype 2 neuraminidase and wherein the first amino acid or first and second amino acids of the head region have been deleted; or the tetrameric neuraminidase of claim 5, wherein the modified monomeric influenza virus subtype 2 neuraminidase lacks amino acids 1-83 or 1-84 of the influenza virus subtype 2 neuraminidase.

11. The modified monomeric influenza virus subtype 2 neuraminidase, the artificial nucleic acid molecule, or tetrameric neuraminidase of any of the preceding claims, wherein the signal peptide is a mammalian signal peptide.

12. The modified monomeric influenza virus subtype 2 neuraminidase, the artificial nucleic acid molecule, or tetrameric neuraminidase of claim 11, wherein the mammalian signal peptide is a CD5 signal peptide.

13. The modified monomeric influenza virus subtype 2 neuraminidase, the artificial nucleic acid molecule, or tetrameric neuraminidase of claim 12, wherein the CD5 signal peptide comprises the amino acid sequence MPMGSLQPLATLYLLGMLVASVLS (SEQ ID NO:132) or MPMGSLQPLATLYLLGMLVASCLG (SEQ ID NO:133).

14. The modified monomeric influenza virus subtype 2 neuraminidase, the artificial nucleic acid molecule, or tetrameric neuraminidase of any of the preceding claims, wherein the modified monomeric influenza virus subtype 2 neuraminidase further comprises a linker sequence that links the head region to the signal peptide or to the C-terminal amino acid of the stalk region.

15. The modified monomeric influenza virus subtype 2 neuraminidase, the artificial nucleic acid molecule, or tetrameric neuraminidase of any of the preceding claims, wherein the linker sequence comprises glycine and/or serine residues and is 2-10 amino acids in length.

16. The modified monomeric influenza virus subtype 2 neuraminidase, the artificial nucleic acid molecule, or tetrameric neuraminidase of any of the preceding claims, wherein the host cell is a mammalian cell.

17. The modified monomeric influenza virus subtype 2 neuraminidase, the artificial nucleic acid molecule, or tetrameric neuraminidase of any of the preceding claims, wherein the influenza virus subtype 2 neuraminidase is from a standard of care influenza strain.

18. The modified monomeric influenza virus subtype 2 neuraminidase, the artificial nucleic acid molecule, or tetrameric neuraminidase of any of the preceding claims, wherein the influenza virus subtype 2 neuraminidase is from a Type A influenza virus.

19. The modified monomeric influenza virus subtype 2 neuraminidase, the artificial nucleic acid molecule, or tetrameric neuraminidase of claim 18, wherein the Type A influenza virus is one of A/Perth/16/2009, A/Kansas/14/2017, A/Belgium/4217/2015, A/Singapore/INFINM-16-0019/2016, A/Switzerland/8060/2017, A/Nevada/32/2013, A/Yamagata/62/1993, A/Nigeria/120/2014, A/Bangkok/i/1979, A/Albany/42/1975, A/Washington/60/2014, A/HongKong/CUHK13510/2001, A/Marrakech/79/2014, A/HongKong/1774/99, A/Illinois/34/2012, A/Kannur/MCVR5404/2010, A/Peru/3216/2016, A/Ohio/13/2017, A/Nanjing/1663/2010, A/Fukuoka/DS7282/2017, A/Ohio/62/2012, A/Minnesota/11/2010, A/Utah/11/2011, A/Hokkaido/10H079/2011, A/Ishikawa/DS7157/2016, A/Gambia/G0071436/2012, A/Kagawa/DS722/2016, A/South_Australia/85/2018, A/Victoria/361/2011, A/Tokyo/DS7334/2017, A/Hochiminh/4596/2010, A/Kagawa/DS7144/2016, A/Peru/4617/2017, A/Switzerland/9715293/2013, A/Tokyo/UTSK1/2007, A/WesternAustralia/13/2001, A/Tasmania/1018/2015, A/Sweden/3/2017, A/Kansas/13/2009, A/Tokyo/DS763/2016, A/Newcastle/67/2016, A/SouthAustralia/34/2019, A/Stockholm/32/2014, A/Stockholm/14/2012, A/Stockholm/15/2014, A/Guangxigangbei/190/2019, A/Xinjiangtianshan/1411/2012, A/NewYork/654/1994, A/Netherlands/620/1989, A/NewYork/758/1993, A/Catalonia/9503S/2017, A/Paris/2379/2014, or A/Heilongjiangxiangyang/1134/2011.

20. The modified monomeric influenza virus subtype 2 neuraminidase, the artificial nucleic acid molecule, or tetrameric neuraminidase of claim 19, wherein the Type A influenza virus is one of A/Perth/16/2009, A/Kansas/14/2017, A/Belgium/4217/2015, A/Singapore/INFIMH-16-0019/2016, A/Switzerland/8060/2017, A/HongKong/4801/2014, A/SouthAustralia/34/2019, A/Victoria/361/2011, or A/HongKong/45/2019.

21. The modified monomeric influenza virus subtype 2 neuraminidase, the artificial nucleic acid molecule, or tetrameric neuraminidase of any of the preceding claims, wherein the head region of the influenza virus subtype 2 neuraminidase is the head region of the influenza virus subtype 2 neuraminidase of any one of SEQ ID NO: 1-115.

22. The modified monomeric influenza virus subtype 2 neuraminidase, the artificial nucleic acid molecule, or tetrameric neuraminidase of any of the preceding claims, wherein the modified monomeric influenza virus subtype 2 neuraminidase comprises an amino acid sequence selected from the group consisting of SEQ ID NO:117-131.

23. A host cell comprising the artificial nucleic acid molecule of any of the preceding claims.

24. The host cell of claim 23, wherein the host cell is a mammalian cell.

25. The host cell of claim 24, wherein the mammalian cell is a Chinese Hamster Ovary (CHO) cell.

26. A vaccine composition, comprising the tetrameric neuraminidase of any of the preceding claims or an artificial nucleic acid molecule encoding the modified monomeric influenza virus subtype 2 neuraminidase of any of the preceding claims.

27. The vaccine composition of claim 26, further comprising an adjuvant.

28. The vaccine composition of claim 26 or 27, further comprising an influenza virus hemagglutinin.

29. The vaccine composition of claim 28, wherein the influenza virus hemagglutinin is from a first influenza strain and the modified influenza virus subtype 2 neuraminidase is from a second influenza strain, wherein the first influenza strain and the second influenza strain are different influenza strains.

30. An in vitro method of producing the tetrameric neuraminidase of any of claims 3-22, wherein the method comprises culturing the host cell of anyone of claims 23-25 in a cell culture medium, and expressing the modified monomeric influenza virus subtype 2 neuraminidase in the host cell, wherein following expression of the modified monomeric influenza virus subtype 2 neuraminidase in the host cell, a host cell supernatant comprises modified influenza virus subtype 2 neuraminidase comprising at least the tetrameric neuraminidase and a monomeric form of the modified monomeric influenza virus subtype 2 neuraminidase.

31. The method of claim 30, wherein the tetrameric neuraminidase represents at least 20%, at least 50%, at least 70%, or at least 90% of the modified influenza virus subtype 2 neuraminidase that is secreted upon expression of the modified monomeric influenza virus subtype 2 neuraminidase in the host cell, as measured by size exclusion chromatography.

32. The method of claim 30 or 31, wherein the host cell is cultured in a volume of 10-10,000 liters of cell culture medium.

33. The method of any one of claims 30-32, further comprising a step of purifying the secreted tetrameric neuraminidase from the cell culture to yield a purified influenza virus subtype 2 tetrameric neuraminidase.

34. The method of claim 33, wherein the purified tetrameric influenza virus subtype 2 neuraminidase is present in an amount of at least 0.5 mg/mL.

35. A method of immunizing a subject against influenza virus comprising administering to the subject an immunologically effective amount of the vaccine composition of any one of claims 26-29.

36. The method of claim 35, wherein the method prevents influenza virus infection in the subject.

37. The method of claim 35, wherein the method raises a protective immune response in the subject.

38. The method of claim 37, wherein the protective immune response is an antibody response.

39. A method of reducing one or more symptoms of influenza virus infection, the method comprising administering to a subject a prophylactically effective amount of the composition of any one of claims 26-29.

40. The method of claim 39, wherein the one or more symptoms of influenza virus infection is a reduction in body weight.

41. The method of claim 40, wherein the one or more symptoms of influenza virus infection is an increase in body temperature.

42. A method of vaccinating a subject against influenza virus, the method comprising administering to the subject an amount of the vaccine composition of any one of claims 26-29 effective to vaccinate the subject against influenza virus.

43. The method of any one of claims 35-42, wherein the subject is a human.

44. The method of any one of claims 35-43, wherein the vaccine composition is administered intramuscularly, intradermally, subcutaneously, intravenously, or intraperitoneally.

45. The method of any one of claims 35-44, wherein the method treats or prevents disease caused by either or both a seasonal or a pandemic influenza strain.

46. The method of any one of claims 35-45, wherein the human subject is 6 months of age or older, less than 18 years of age, at least 60 years of age, at least 65 years of age, at least 6 months of age and less than 18 years of age, or at least 18 years of age and less than 65 years of age.


SEQUENCE LISTING (138 sequences)

SEQ ID NO: 1 - protein, 469 aa, Influenza A virus
MNPNQKIITIGSVSLTISTICFFMQIAILITTVTLHFKQYEFNPPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLNLMPI

SEQ ID NO: 2 - protein, 469 aa, Influenza A virus
MNPNQKIITIGSVSLTIATVCFLMQIAILVTTVTLHFRQYECDSPANNQVMPCEPIIIERNITEIVYLNNTTIEKEICPKLVEYRNWSKPQCKITGFAPFSKDNSIRLSAGGDIWVTREPYVSCDPGKCYQFALGQGTTLDNKHSNDTIHDRIPHRTLLMNELGVPFHLGTRQVCIAWSSSSCHDGKAWLHVCVTGDDKNATASFIYDGKLVDSIGSWSQNILRTQESECVCINGTCTVVMTDGSASGRADTRILFIEEGKIVHISPLSGSAQHVEECSCYPRYPGVRCICRDNWKGSNRPVVDINVEDYSIDSSYVCSGLVGDTPRNNDRSGNSNCRNPNNERGNHGVKGWAFDDGNDVWMGRTINKDLRSGYETFKVIGGWSTPNSKSQINRQVIVDSDNRSGYSGIFSVEGKSCINRCFYVELIRGREQETRVWWTSNSIVVFCGTSGTYGTGSWPDGADINLMPI

SEQ ID NO: 3 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDRCYQFALGQGTTLNNMHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSRYVCSGLVGDTPRKNDSSSSSHCLDPNNEEGGHGVKGWAFDDGNDVWMGRTINETSRLGYETFKVIEGWSNPKSKLQTNRQVLVDRGDRSGYSGIFSVEGKSCINRCFYVELIRGRKEETEVLWTSNSIVVFCGTSGTYGTGSWPDGADLNLMPI

SEQ ID NO: 4 - protein, 469 aa, Influenza A virus
MNPNQKIITIGSVSLTIATICFLMQIVILVTTVTLHFKQYECNSPPNNQVMLCEPTIIERNITEIVYLTNTTIEKEICPKLAEYRNWSKPQCKITGFAPFSKDNSIRLSAGGDIWVTREPYVSCDPDKCYQFALGQGTTLNNRHSNDIVHDRTPYRTLLMNELGVPFHLGTKQVCIAWSSSSCHDGKAWLHVCVTGHDENATASFIYDGRLVDSIGSWSKKILRTQESECVCINGTCTVVMTDGSASGRADTKILFIEEGKIVHISPLSGSAQHVEECSCYPQYSGVRCVCRDNWKGSNRPIVDINVKDYSIVSSYVCSGLVGDTPRKNDSSSSSHCLNPNNEEGGHGVKGWAFDDGNDVWMGRTISEKLRSGYETFKVIGGWSKPNSKLQINRQVIVDRGNRSGYSGIFSVEGKNCINRCFYVELIRGRKQETEVWWTSNSIVVFCGTSGTYGTGSWPDGADINLMPI

SEQ ID NO: 5 - protein, 469 aa, Influenza A virus
MNPNQKIITIGSVSLTISTICFFMQIAILITTVTLHFKQYEFNSPPNNQVVLCEPTIIERNITEIVYLTNTTIEKEICPKPAEYRNWSKPQCGITGFAPFSKDNSIRLSAGGDIWVTREPYVSCDPDKCYQFALGQGTTLNNGHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIIHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEENEVLWTSNSIVVFCGTSGTYGTGSWPDGADLNLMPI

SEQ ID NO: 6 - protein, 469 aa, Influenza A virus
MNPNQKIITIGSVSLTIATICFLMQIAILVTTVTLHFKQYECDSPANNQVMPCEPIIIERNITEIVYLTNTTIEKEMCPKLVEYRNWSKPQCKITGFAPFSKDNSIRLSAGGDIWVTREPYVSCDPGKCYQFALGQGTTLNNRHSNDTIHDRIPHRTLLMNELGVPFHLGTRQVCIAWSSSSCHDGKAWLHVCVTGDDKNATASFIYDGRLVDSIGSWSQNILRTQESECVCINRTCTVVMTDGSASGKADTRILFIEEGKIVHISPLSGSAQHVEECSCYPRYPGVRCICRDNWKGSNRPVVDINVKDYSIDSSYVCSGLVGDTPRNNDRSSSSYCRNPNNERGNHGVKGWAFDDENDVWMGRTISKDSRSGYETFKVIGGWSTPNSKSQINRQVIVDSDNRSGYSGIFSVEGKSCINRCFYVELIRGREQETRVWWTSNSIVVFCGTSGTYGTGSWPDGADINLMPI

SEQ ID NO: 7 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTINNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 8 - protein, 471 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVISWSKDILRTQESECVCINGTCTVVMTDGSASGEADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQMNRQVIVDRGDRSGYSGIFSVESKSCINRCFYVELIRGRKEETEVLWTSNSIVVFCGTSGTYGTGSWPDGADPSGHEPVP

SEQ ID NO: 9 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMSELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGVHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMLI

SEQ ID NO: 10 - protein, 469 aa, Influenza A virus
MNPNQKIITIGSVSLTISTICFFMQIAILITTVTLHFKQYEFNSPPNNQVMLCEPTIIERNITEIVYLTNTTIEKEVCPKPAEYRNWSKPQCGITGFAPFSKDNSIRLSDGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIASSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSIEGKSCINRCFYVELIRGRKEETEVLWTSNSIVVFCGTSGTYGTGSWPDGADLNLMPI

SEQ ID NO: 11 - protein, 468 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCVCRDNWKGSNRPIVDINMEDHSIVSSYVCSGLVGDTPRKNDSSSSSHCLNPNNERGHGVKGWAFDDGNDVWMGRTINEDSRLGYETFKVIGGWSTPNSKSQINRQVIVDSDNRSGYSGIFSVEGKSCINRCFYVELIRGRKQETRVWWTSNSIVVFCGTSGTYGTGSWPDGADLNLMLI

SEQ ID NO: 12 - protein, 468 aa, Influenza A virus
MNPNQKIITIGSVSLTISTICFFMQIAILITTVTLHFKQYEFNSPPNNQVMLCEPTVIERNITEIVYLTNTTIEKEICPKPAEYRNWSKPQCQITGFAPFSKDNSIRLSAGGDIWVTREPYVSCDPDKCYQFALGQGTTINNVHSNNTARARTPYRTLLMNELGVPFHLGTKQVCIAWSSSSCHDGKAWLHVCITGDDKNATASFIYDGRLVDSIGSWSQNILRTQESECVCINGTCTVVMTDGNATGKADTKILFIEEGKIVHTSKLSGSAQHVEECSCYPRYPGVRCVCRDNWKGSNRPIVDINMEDHSIVSSYVCSGLVGDTPRKNDSSSSSHCLNPNNERGHGVKGWAFDDGNDVWMGRTINEDSRLGYETFKVVEGWSTPNSKSQINRQVIVDSDNRSGYSGIFSVEGKSCINRCFYVELIRGRKQETRVWWTSNSIVVFCGTSGTYGTGSWPDGADLNLMHI

SEQ ID NO: 13 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCQITGFAPFSKDNSIRLSASGDIWVTREPYVSCDPDKCYQFALGQGTTINNVHSNNTARDRTPYRTLLMNELGVPFHLGTKQVCIAWSSSSCHDGKAWLHVCITGDDKNATASFIYNGRLVDSVVSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGADLNLMHI

SEQ ID NO: 14 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCAVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGNRSGYSGIFSVEGKNCINRCFYVELIRGRKEETEVLWTSNSIVVFCGTSGTYGTGSWPDGADLNLMPM

SEQ ID NO: 15 - protein, 468 aa, Influenza A virus
MNPNQKIITIGSVSLTISTICFFMQIAILITTVTLHFKQYEFNPPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMSELGVPFHLGTKQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCLNPNNERGHGVKGWAFDDGNDVWMGRTINEDSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMLI

SEQ ID NO: 16 - protein, 469 aa, Influenza A virus
MNPNQKIITVGSISLTISIICFFMQIAILITTVTLHFKRYEFNSPPNNQVMLCEPTIIERNITEIVYLTNTTIEKEICPKLAEYRNWSKPQCDITGFAPFSKDNSIRLSAGGDIWVTREPYVSCDTDKCFQFALGQGTTLNNVHSNDTVHDRTPYRTLLMSELGVPFHLGTKQVCIAWSSSSCHDGKAWLHVCVTGDDKNATASFIYNGRLVDSVVSWSKKILRTQESECVCINGICTVVMTDGSASGKADTKILFIKEGKIVHTSTLSGSAQHVEECSCYPRYPGVRCVCRDNWKGSNRPIVDINIKDYSTVSSYVCSGLVGDTPRNPDSSSSSHCLDPNNEEGGHGVKGWAFDDGNDVWMGRTISEKSRLGYETFKVIGGWFNPNSKSQINRQVIVDKGNRSGYSGIFSVEGKSCINRCFYVELIRGRKDETEVLWTSNSIVVFCGTSGTYGTGSWPDGADIKLMSV

SEQ ID NO: 17 - protein, 469 aa, Influenza A virus
MNPNQRIITIGSVSLIIATVCFLMQIAILVITVTLHFKQHDCNFSPNNQLMQCEPTIIERNMTEIVYLTNTTIEKEICPKLAEYRNWSKPQCHITGFAPFSKDNSVRLSAGGDIWVTREPYVSCDPDKCYQFALGQGTTLNNGHSNDTVHDRTPYRTLLMNELGVPFHLGTRQVCIAWSSSSCHDGKAWLHVCITGNDNNATASFIYNGRLVDSIGSWSKNILRTQESECVCINGTCTVVMTDGSASGKADTKILFVEEGKIVHISTLSGSAQHVEECSCYPRYPGVRCVCRDNWKGSNRPIVDINVKDYSTTSSYVCSGLVGDTPRKNNSFSSSRCINPNNEEGGHGVKGWAFDDGDDVWMGRTISEKSRLGYETFKVIEGWSKPNSKVQTNRQVIVERGDRSGYSGIFSVEGKNCINRCFYVELIRGRKEEIKVWWTSNSIVVFCGTSGTYGTGSWPDGADINLMPI

SEQ ID NO: 18 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCQITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSQNILRTQESECVCINGTCTVVMTDGSASGRADTRILFIEEGKIVHISPLSGSAQHVEECSCYPRYPGVRCVCRDNWKGSNRPIVDINMEDHSIVSSYVCSGLVGDTPRKNDSSSSSHCRNPNNERGNQGVKGWAFDNGDDVWMGRTISKDLRSGYETFKVIGGWSTPNSKSQINRQVIVDSDNRSGYSGIFSVEGKSCINRCFYVELIRGRKEETEVLWTSNSIVVFCGTSGTYGTGSWPDGADLNLMHI

SEQ ID NO: 19 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCSITGFAPFSKDNSIRLSAGGDIWVTREPYVSCDPDKCYQFALGQGTTLNNVHSNNTIRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSKLSGSAQHVEECSCYPRYPGVRCVCRDNWKGSNRPIVDINIKDHSIVSSYVCSGLVGDTPRKNDSSSSSHCSDPNNEEGGHGVKGWAFDDGNDVWMGRTINETSRLGYETLKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 20 - protein, 469 aa, Influenza A virus
MNPNQKIITIGSVSLTISTICFFMQIAILITTVTLHFKQYEFNSPPNNQMMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 21 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPNKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINMKDHSIVSSYVCSGLVGDTPRKNDSSSSSHCFDPNDEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 22 - protein, 469 aa, Influenza A virus
MNPNQKIITIGSVSLTIATICFLMQIAILVTTVTLHFKQYECSSPPNNQVMLCEPTIIERNITEIVYLTNTTIEKEICPKLAEYRNWSKPQCKITGFAPFSKDNSIRLSAGGDIWVTREPYVSCDPDKCYQFALGQGTTLNNRHSNDTVHDRTPYRTLLMNELGVPFHLGTKQVCIAWSSSSCHDGKAWLHVCVTGHDENATASFIYDGRLVDSIGSWSKKILRTQESECVCINGTCTVVMTDGSASGRADTKILFIEEGKIVHISPLSGSAQHVEECSCYPRYPGVRCVCRDNWKGSNRPIVDINVKDYSIVSSYVCSGLVGDTPRKNDSSSSSHCLNPNNEEGGHGVKGWAFDDGNDVWMGRTISEKFRSGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 23 - protein, 469 aa, Influenza A virus
MNPNQKIITIGSVSLTIATICFLMQIAILVTTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNRHSNDTVHDRTPYRTLLMNELGVPFHLGTKQVCIAWSSSSCHDGKAWLHVCVTGHDENATASFIYDGRLVDSIGSWSKKILRTQESECVCINGTCTVVMTDGSASGRADTKILFIEEGKIVHISPLSGSAQHVEECSCYPRYPGVRCVCRDNWKGSNRPIVDINVKDYSIVSSYVCSGLVGDTPRKNDSSSSSHCLNPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 24 - protein, 469 aa, Influenza A virus
MNPNQKIITIGSVSLTISRICFFMQIAILINTVTLHFKQYEFNSPPNNQVMLCEPTIIERNITKIVYLTNTTIEKEICPKLAEYRSWSKPQCDITGFAPFSKDNSIRLSAGGDIWVTREPYVSCDPDKCFQFALGQGTTLNNVHSNDTVHDRTPYRTLLMNELGVPFHLGTKQVCIAWSSSSCHDGKAWLHVCITGDDKNATASIIYNGRLVDSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGFVGDTPRKPDRSSSSHCLDPNNEEGGHGVKGWAFDDGNDVWMGRTISEKSRLGYETFKVIGGWSNPNSKSQINRQVIVDRGNRSGYSGIFSVEGKSCINRCFYVELIRGRKDETEVLWTSNSIVVFCGTSGTYGTGSWPDGADINLMPI

SEQ ID NO: 25 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSFSSSHCLDPNNEEGGHGVKGWAFDDGNDVWMGRTINETSRLGYETFKVIEGWSNPKSKLQINRQVIVDRGNRSGYSGIFSVEGKSCINRCFYVELIRGRKEETEVLWTSNSIVVFCGTSGTYGTGSWPDGADLNLMPI

SEQ ID NO: 26 - protein, 469 aa, Influenza A virus
MNPNQKIITIGSVSLTIATVCFLMQIAILVTTVTLHFKQYECDSPANNQVMPCEPIIIERNITEIVYLTNTTIEKEICPKLVEYRNWSKPQCKITGFAPFSKDNSIRLSAGGDIWVTREPYVSCDPGKCYQFALGQGTTLDNKHSNDTIHDRIPHRTLLMNELGVPFHLGTRQVCIAWSSSSRHDGKAWLHVCVTGDDKNATASFIYDGRLVDSIGSWSQNILRTQESECVCINGTCTVVMTDGSASGRADTRILFIEEGKIVHISPLSGSAQHVEECSCYPRYPGVRCVCRDNWKGSNRPVADINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGADINFMPI

SEQ ID NO: 27 - protein, 469 aa, Influenza A virus
MNPNQKIITIGSVSLTIATVCFLMQIAILVTTVTLHFKQYECDSPANNQVMPCEPIIIERNITEIVYLTNTTIEKEICPKLVEYRNWSKPQCKITGFAPFSKDNSIRLSAGGDIWVTREPYVSCDPGKCYQFALGQGTTLDNKHSNDTIHDRIPHRTLLMNELGVPFHLGTRQVCIAWSSSSCHDGKAWLHVCVTGDDENATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINVKDYSIDSRYVCSGLVGDTPRNNDKSSNSNCRNPNNDKGNHGVKGWAFDDGNDVWMGRTISKDSRSGYETFKVIGGWSTPNSKSQINRQVIVDSDNRSGYSGIFSVEGKSCINRCFYVELIRGREQETKVWWTSNSIVVFCGTSGTYGTGSWPDGADINLMSI

SEQ ID NO: 28 - protein, 469 aa, Influenza A virus
MNPNQKIITIGSVSLTIATMCLLMQIAILATTITLHFKQYECDSPANNQVMPCEPTIIKKNITEIVYLTNTTIEKEVCPKLVEYRNWSKPQCKITGFAPFSKDNSIRLSAGGAVWVTREPYVSCDPSRCYQFALGQGTTLDNRHSNDTIHDRTPYRTLLMNELGVPFHLGTRQVCIAWSSSSCHDGKAWLHVCVTGHDKNATASFIYDGKLVDSIGSWSQNILRTQESECVCINGTCTVVMTDGSASGKADTKIIFIKEGKIIHISPLSGSAQHVEECSCYPRYPGVRCICRDNWKGSNRPVVDINVKDYSIDSSYVCSGLVGDTPRNNDGSSNSNCRNPNNERGNHGVKGWAFDDGNDIWMGRTISKDSRSGYETFKVIGGWSKSNSKFQINRQVIVDSDNRSGYSGVFSVESKSCINRCFYVELIRGRRQENRVWWTSNSIVVFCGTSGTYGSGSWPDGADINLMPI

SEQ ID NO: 29 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDKGDRSGYSGIFSVEGKSCINRCFYVELIRGRKEETEVLWTSNSIVVFCGTSGTYGTGSWPDGADLNLMHI

SEQ ID NO: 30 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 31 - protein, 469 aa, Influenza A virus
MNPNQKIITIGSVSLTIATICFLMQIAILVTTVTLHFKQYECNSPPNNQVMLCEPTIIERNITEIVYLTNTTIEKEICPKLAAYRNWSKPQCNITGFAPFSKDNSIRLSAGGDIWVTRELYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVRISPLSGSAQHVEECSCYPRYPGVRCVCRDNWKGSNRPIVDINVKDYSIVSSYVCSGLVGDTPRKNDSFSSSHCLDPNNEEGGHGVKGWAFDDGNDVWMGRTISEKLRSGYETFKVIEGWSKPNSKLQVNRQVIVDSGNRSGYSGIFSVEGKSCINRCFYVELIRGRKQETEVWWTSNSIVVFCGTSGTYGTGSWPDGADINLMPI

SEQ ID NO: 32 - protein, 469 aa, Influenza A virus
MNPNQKIITIGSVSLTISTICFFMQIAILITTVTLHFKQYEFNSLPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 33 - protein, 469 aa, Influenza A virus
MNPNQKIITIGSVSLTIATICFLMQIAILVTTVTLHFKQYECDYPANNQAMPCEPIIIERNITEIVYLTNTTIEKEVCPKLVEYRNWSKPQCKITGFAPFSKDNSIRLSAGGDIWVTREPYVSCDPGKCYQFALGQGTTLDNKHSNDTIHDRTPHRTLLMNELGVPFHLGTRQVCIAWSSSSCHDGKAWLHVCVTGYDKNATASFIYDGRLVDSIGSWSQNILRTQESECVCINGTCTVVMTDGSASGRADTKILFIEEGKIVHTSPLSGSAQHVEECSCYPRYPGVRCICRDNWKGSNRPVVDINVKDYSINSSYVCSGLVGDTPRNNDRSSSSYCQNPNNEKGTHGVKGWAFDDGNDVWMGRTISEDSRSGYETFKVIGGWSTPNSKLQINRQVIVDSDNRSGYSGIFSVEGKSCINRCFYVELIRGREQETRVWWTSNSIVVFCGTSGTYGTGSWPDGADINIMPI

SEQ ID NO: 34 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLDKYRNWSKPQCDITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKKILMTQESECVCINGTCTVVMTDGSASGKADTKILFIEEGKIVHTSTLSGSAQHVEECSCYPRYPGVRCVCRDNWKGSNRSIVDINIKDYSIVSSYVCSGLVGDTPRKNDSSSSSHCLDPNNEEGGHGVKGWAFDDGNDVWMGRTISEKLPSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 35 - protein, 469 aa, Influenza A virus
MNPNQKIITIGSVSLIIATICFLMQIAILVTTVTLHFKQHDYNSPPNNQAMLCEPTIIERNTTEIVYLTNITIEKEICPKLAEYRNWSKPQCNITGFAPFSKDNSIRLSAGGDIWVTREPYVSCDPDKCYQFALGQGTTLNNGHSNNTVHDRTPYRTLLMNELGVPFHLGTRQVCMAWSSSSCHDGKAWLHVCITGNDNNATASFIYNGRLVDSIGSWSKNILRTQESECVCINGTCTVVMTDGSASGKADTKILFVEEGKIVHISTLSGSAQHVEECSCYPRFPGVRCVCRDNWKGSNRPIVDINVKNYSIVSSYVCSGLVGDTPRKSDSVSSSYCLDPNNEKGGHGVKGWAFDDGNDVWMGRTINETLRLGYETFKVIEGWSKANSKLQTNRQVIVEKGDRSGYSGIFSVEGKSCINRCFYVELIRGRKEETKVWWTSNSIVVFCGTSGTYGTGSWPDGADINLMPI

SEQ ID NO: 36 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPVKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSRYVCSGLVGDTPRKNDSSSSSHCLNPNNEEGGHGVKGWAFDDGNDVWMGRTINETSRFGYETFKVVEGWSNPKSKLQINRQVIVDRDDRSGYSGIFSVEGKSCINRCFYVELIRGRKEETEVLWTSNSIVVFCGTSGTYGTGSWPDGADLNLMHI

SEQ ID NO: 37 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCQITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSQNILRTQESECVCINGTCTVVMTDGNATGKADTKILFIEEGKIVHTSKLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKEETEVLWTSNSIVVFCGTSGTYGTGSWPDGANINFMPI

SEQ ID NO: 38 - protein, 469 aa, Influenza A virus
MNPNQKIITIGSVSLTISTICFFMQIAILTTTVTLHFRQYEFNSPPNNQVMLCEPTIIERNITEIVYLTNTTIEKEICPKPAEYRNWSKPQCQITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSQNILRTQESECVCINGTCTVVMTDGNATGKADTKILFIEEGKIVHTSTLSGSAQHVEECSCYPRYPGVRCICRDNWKGSNRPVVDINMEDYSIDSSYVCSGLVGDTPRNDDRSSNSNCRNPNNERGNQGVKGWAFDDGNDVWMGRTINEDSRLGYETFKVVEGWSNPKSKLQINRQVIIDRGDRSGYSGIFSVEGKSCINRCFYVELIRGRKQETRVWWTSNSIVVFCGTSGTYGTGSWPDGANINFMPI

SEQ ID NO: 39 - protein, 468 aa, Influenza A virus
MNPNQKIITIGSVSLTISTICFFMQIAILIITVTLHFKQYEFNSPPNNQVMLCEPTIIERNITEIVYLTNTTIEKEICPKPAEYRDWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYDGRLVDSIGSWSQNILRTQESECVCINGTCTVVMTDGSASGRADTRILFIEEGKIVHTSKLSGSAQHVEECSCYPRYPGVRCVCRDNWKGSNRPIVDINMEDHSIDSSYVCSGLVGDTPRKNDSSSSSHCLNPNNERGHGVKGWAFDDGNDVWMGRTINEDSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 40 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMSELGVPFHLGTKQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKEETEVLWTSNSIVVFCGTSGTYGTGSWPDGADLNLMLI

SEQ ID NO: 41 - protein, 469 aa, Influenza A virus
MNPNQKIITIGSVSLTISTICFFMQIAILITTVTLHFKQNEFNSPPNNQVMLCEPTIIERNITEIVYLNNTTIEKEICPKVVEYRNWSKPQCQITGFAPFSKDNSIRLSAGGDIWVTREPYVSCDPDKCYQFALGQGTTLNNVHSNNTVRDRTPYRTLLMSELGVPFHLGTKQVCIAWSSSSCHDGKAWLHVCITGDDKNATASFIYNGRLVDSVVSWSQNILRTQESECVCINGTCTVVMTDGNATGKADTKILFIEEGKIVHTSKLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVVEGWSNPKSKLQINRQVIVDRGDRSGYSGIFSVEGKSCINRCFYVELIRGRKEETEVLWTSNSIVVFCGTSGTYGTGSWPDGADLNLMLI

SEQ ID NO: 42 - protein, 468 aa, Influenza A virus
MNPNQKIITIGSVSLTISTICFFMQIAILVTTVTLHFKQYECDSPASNQVMPCEPIIIERNITEIVYLNNTTIEKEICPKVVEYRNWSKPQCQITGFAPFSKDNSIRLSAGGDIWVTREPYVSCDPDKCYQFALGQGTTINNVHSNNTARNRTPYRTLLMNELGVPFHLGTKQVCIAWSSSSCHDGKAWLHVCITGDDKNATASFIYNGRLVDSVVSWSQNILRTQESECVCINGTCTVVMTDGNATGKADTKILFIEEGKIVHTSKLSGSAQHVEECSCYPRYPGVRCVCRDNWKGSNRPIVDINMEDHSIVSSYVCSGLVGDTPRKNDSSSSSHCLNPNNERGHGVKGWAFDDGNDVWMGRTINEDSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 43 - protein, 469 aa, Influenza A virus
MNPNQKIITIGSVSLTISTICFFMQIAILITTVTLHFKQYEFNSPPNNQVMLCEPTIIERNITEIVYLNNTTIEKEICPKVVEYRNWSKPQCQITGFAPFSKDNSIRLSAGGDIWVTREPYVSCDPDKCYQFALGQGTTINNVHSNNTARDRTPYRTLLMNELGVPFHLGTKQVCIAWSSSSCHDGKAWLHVCITGDDKNATASFIYDGRLVDSIGSWSQNILRTQESECVCINGTCTVVMTDGNATGKADTKILFIEEGKIVHTSKLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGADLNLMHI

SEQ ID NO: 44 - protein, 469 aa, Influenza A virus
MNPNQKIITIGSVSLIIATICFLMQIAILVTTVTLHFKQHDCNSPPNNQVMLCEPTIIERNTTEIVYLTNTTIEKEICPKLAEYRDWSKPQCNITGFAPFSKDNSIRLSAGGDIWVTREPYVSCDPDKCYQFALGQGTTLDNGHSNDTVHDRTPYRTLLMNELGVPFHLGTRQVCIAWSSSSCHDGKAWLHVCITGDDKNATASFIYNGRLVDSIGSWSKNILRTQESECVCINGTCTVVMTDGSASGKADTKILFIEEGKIVHISTLSGSAQHVEECSCYPRYPGVRCVCRDNWKGSNRPIVDINVKDYSIVSSYVCSGLVGDTPRKNDSFSSSHCLNPNNEEGGHGVKGWAFDDGNDVWMGRTISDKLRSGYETFKVIEGWSKPNSKLQTNRQVIVERGKRSGYSGIFSVEGKSCINRCFYVELIRGRKEETKVWWTSNSIVVFCGTSGTYGTGSWPDGADINLMPI

SEQ ID NO: 45 - protein, 469 aa, Influenza A virus
MNPNQKIITIGSVSLTISTICFFMQIAILITTVTLHFKQYEFNSPPNNQVMLCEPTIIERNITEIVYLTNTTIEREICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 46 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEVLRTQESECVCINGTCTVVMTDGSASGKADTKILFIEEGKIVHTSTLSGSAQHVEECSCYPRYPGVRCVCRDNWKGFNRPIVDINIKDHSIVSSYVCSGLVGDTPRKNDRSSRSPCLDPNNEEGGHGVKGWAFDDGNDVWMGRTINGTSRLGYETFKVIEGWFNPKFKLQINRQVIVDRGDRSGFFGIFFVEGKKCIHWWFFVELIRGRKEENEVLWTSNRIVVFCGTSGTYGTGSWPDGADLNFMPI

SEQ ID NO: 47 - protein, 469 aa, Influenza A virus
MHPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGKTLNNVHSNNTVRDRTPYRTLLMNELGVPFHLGTKQVCIAWSSSSCHDGKAWLHVCITGDDKNATASFIYNGRLVDSVVSWSKEVLRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRIIGISTKSRSKDRIQIEGYQGVNGFLSPDANDVWSGRTIYEKSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 48 - protein, 469 aa, Influenza A virus
MNPNQKIITIGSVSLTIATICFLMQIAILVTTVTLHFKQYECNSPPNNQVMLCEPTIIERNITEIVYLTNTTIEKEICPKLAEYRNWSKPQCNITGFAPFSKDNSIRLSAGGDIWVTREHYVSCDRDKCYQFALGQGTTLNNGHSNDTVHDRTPYRTLLMNELGVPFHLGTKQVCIAWSSSSCHHGKAWLHVCVTGDDENATASFIYNGRLVDSIGSWSKKILRTQESECVCINGTCTVVMTDGSASGKADTKILFIKEGKIVHTSSLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKANSKLQINRQVIVDRGNRSGYSGIFSVESKSCINRCFYVELIRGRKQETEVLWTSNSIVVFCGTSGTYGTGSWPDGADINLMPI

SEQ ID NO: 49 - protein, 469 aa, Influenza A virus
MNPNQKIITIGSVSLTIATICFLMQIAILVTTVTLHFKQYECDSPANNQVMPCEPIIIERNITEIVYLTNTTIEKEICPKLVEYRNWSKPQCKITGFAPFSKDNSIRLSAGGDIWVTREPYVSCDPGRCYQFALGQGTTLDNKHSNDTIHDRTPHRTLLMNELGVPFHLGTRQVCIAWSSSSCHDGKAWLHVCVTGYDKNATASFIYDGRLVDSIGSWSKNILRTQESECVCINGTCTVVMTDGSASERADTKVLFIEEGKIVHISPLSGSAQHVEECSCYPRYPGVRCVCRDNWKGSNRPVVDINVKDYSIASSYVCSGLVGDTPRKNDRYSSSYCRNPNNEKGNHGVKGWAFDDGNDVWMGRTISDESRSGYETFKVIGGWSTPNSKLQINRQVIVDSDNRSGYSGIFSVEGKSCINRCFYVELIRGREQETRVWWTSNSIVVFCGTSGTYGTGSWPDGADINLMPI

SEQ ID NO: 50 - protein, 469 aa, Influenza A virus
MNPNQKIITIGSVSLIIATICFLMQIAILVTTVTLHFKQHDYNSPPNNQVMLCEPTIIERNTTEIVYLTNITIEKEICPKLAEYRNWSKPQCNITGFAPFSKDNSIRLSAGGDIWVTREPYVSCDPDKCYQFALGQGTTLNNRHSNNTVHDRSPYRTLLMNELGVPFHLGTRQVCIAWSSSSCHDGKAWLHVCITGDDNNATASFIYNGRLVDSIGSWSKNILRTQESECVCINGTCTVVMTDGSASGKADTKILFIEEGKIVHISTLSGSAQHVEECSCYPRYPGVRCVCRDNWKGSNRPIVDINMKDYSIASSYVCSGLVGDTPRKNDSFSSSHCLDPNNEKGGHGVKGWAFDDGNDVWMGRTISEEFRLGYETFKVIKGWSNPNSKLQTNRQVIVEKGNRSGYSGVFSIEDKSCINRCFYVELIRGRKEETKVWWTSNSIVVFCGTSGTYGTGSWPDGADINLMPI

SEQ ID NO: 51 - protein, 469 aa, Influenza A virus
MNPNQKIITIGSVSLTIATICFLMQIAILVTTVTLHFKPYECSSPPNNQVMPCEPIIIERNITEIVYLTNTTIEKEICPKLVEYRNWSKPQCKITGFAPFSKDNSIRLSAGGDIWVTREPYVSCDPGKCYQFALGQGTTLNNKHSNDTIHDRTPYRTLLMNELGVPFHLGTKQVCIAWSSSSCHDGKAWLHVCVTGHDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPIVDINVKDYSIVSSYVCSGLVGDTPRKNDRSSSSYCRNPNNEKGSHGVKGWAFDDGNDVWMGRTIGEELRSGYETFKVIGGWSTPNSKLQINRQIIVDSKSRSGYSGIFSVEGKSCINRCFYVELIRGREQETRVWWTSNSIVVFCGTSGTYGTGSWPDGADINLMPI

SEQ ID NO: 52 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWTKPQCDITGFAPFSKDNSIRLSAGGDIWVTREPYVSCDPDKCYQFALGQGTTLNNVHSNNTARDRTPYRTLLMSELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSGYVCSGLVGDTPRKNDSSSSSHCLNPNNEEGVHGVKGWAFDEGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMLI

SEQ ID NO: 53 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCGPDKCYQFALGQGTTLNNVHSNNTVRDRTPYRTLLMNELGVPFHLGTKQVCIAWSSSSCHDGKAWLHVCITGDDKNATASFIYNGKLVDSVVSWSKDILRTQESECVCINGTCTVVMTDGNATGKADTKILFIEEGKIVNTSKLSGSAQHVEECSCYPRYPGVRCVCRDNWKGSNRPIVDINIKDHSIVSSYVCSGLVGDTPRKNDSSSSSHCLNPNNEKGGHGVKGWAFDDGNDVWMGRTINETSRLGYETFKVVEGWSNHKSKLQINRQVIVDRGDRSGYSGIFSVEGKSCINRCFYVELIRGRKEETEVLWTSNSIVVFCGTSGTYGTGSWPDGADLNLMHI

SEQ ID NO: 54 - protein, 469 aa, Influenza A virus
MNPNQKIITIGSVSLTIATVCFLTQIAILVTTVTLHFKQYECDSPASNQVMPCEPIIIERNITEIVYLNNTTIEKEICPKVVEYRNWSKPQCQITGFAPFSKDNSIRLSAGGDIWVTREPYVSCDHGKCYQFALGQGTTLNNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGNASGRADTRILFIEEGKIVHISPLSGSAQHVEECSCYPRYPGVRCICRDNWKGSNRPVVDINMEDYSIDSSYVCSGLVGDTPRNDDRSSSSNCRNPNNETGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 55 - protein, 469 aa, Influenza A virus
MNPNQKIITIGSVSLTIATICFLMQIAILATTVTLHFKQNECSIPSNNQVVPCEPIIVERNITEIVYLNNTTIEKELCPKISEYRDWSKPQCQITGFAPFSKDNSIRLSAGGDIWVTREPYVSCSPDKCYQFALGQGTTLDNKHSNGTIHDRIPHRTLLMNELGVPFHLGTKQVCIAWSSSSCHDGKAWLHVCVTGDDRNATASFIYGGMLVDSIGSWSQNILRTQESECVCINGTCTVVMTDGSASGRADTRILFVKEGKIVHISPLSGSAQHIEECSCYPRYPDVRCVCRDNWKGSNRPIIDINMADYSIGSSYVCSGLVGDTPRNDDSSSNSNCRDPNNERGNPGVKGWAFDYGNDVWMGRTISKDSRSGYETFRVIGGWTIANSKSQVNRQVIVDNSNWSGYSGIFSVEGKSCINRCFYVELIRGRPQEARVWWTSNSIVVFCGTSGTYGTGSWPDGANINFMPI

SEQ ID NO: 56 - protein, 469 aa, Influenza A virus
MNPNQKIITIGSVSLTLATICFLMQIAILVTTVTLHFKQYECSSPPNNQVMPCEPTIIERNITEIVYLTNTTIEKEICPKLVEYRNWSKPQCKITGFAPFSKDNSIRLSAGGDIWVTREPYVSCDPSKCYQFALGQGTTLNNRHSNDTVHDRTPYRTLLMNELGVPFHLGTRQVCMAWSSSSCHDGKAWLHVCVTGHDENATASFIYDGRLVDSIGSWSKNVLRTQESECVCINGTCTVVMTDGSASERADTKILFIEEGKIVHISPLSGNAQHVEECSCYPRYPGVRCVCRDNWKGSNRPIVDINVKDYSIVSSYVCSGLVGDTPRKNDRSSSSYCRNPNNEKGSHGVKGWAFDDGNDVWMGRTIGEELRSGYETFKVIGGWSTPNSKLQINRQVIVDSGNRSGYSGIFSVEGKSCINRCFYVELIRGRKQETRVWWTSNSIVVFCGTSGTYGTGSWPDGADINLMPI

SEQ ID NO: 57 - protein, 469 aa, Influenza A virus
MNPNQKIITIGSVSLTITTICFLMQIAILVITVTLHFKQCECNSPPNNQVMLCEPTIIERNITEIVYLTNTTIEKEICPKLAEYRNWSKPQCKITGFAPFSKDNSIRLSAGGDIWVTREPYVSCDPGKCYQFALGQGTTLNNRHSNNTAHDRTPYRTLLMNELGVPFHLGTKQVCIAWSSSSCHDGKAWLHVCVTGHNENATASFIYDGRLVDSIGSWSKNILRTQESECVCINGTCTVVMTDGSASGRADTKILFIKEGKIVHISPLSGSAQHVEECSCYPRYPGVRCVCRDNWKGSNRPIVDINVKDYSIVSSYVCSGLVGDTPRKNDSSSSSYCLDPNNEEGGHGVKGWAFDDGNDVWMGRTISEKLRSGYETFKVIGGWSKPNSKLQINRQVIVDKGNRSGYSGIFSVEGKSCINRCFYVELIRGRKQETEVWWTSNSIVVFCGTSGTYGTGSWPDGADINLMPI

SEQ ID NO: 58 - protein, 469 aa, Influenza A virus
MNPNQKIITIGSVSLTIATICFLMQIAILVTTVTLHFKQYECNSPPSNQAMLCEPTIIERNITEIVYLTNTTIEKEICPKLAEYRNWSKPQCKITGFAPFSKDNSIRLSAGGDIWVTREPYVSCDPGKCYQFALGQGTTINNRHSNDTVHDRTPYRTLLMNELGVPFHLGTKQVCIAWSSSSCHDGKAWLHVCVTGHDENATASFIYDGRLVDSIGSWSKNILRTQESECVCINGTCTVVMTDGSASGRADTKILFIEEGKIVHISPLSGSAQHVEECSCYPRYPGVRCVCRDNWKGSNRPIVDINVKDYSIVSSYVCSGLVGDTPRKNDSSSNSYCRNPNNEKGSHGVKGWAFDDGNDVWMGRTISAESRSGYETFKVIGGWSKPNSKLQINRQVIVDRGNRSGYSGIFSVEGKSCINRCFYVELIRGRKQETEVWWTSNSIVVFCGTSGTYGTGSWPDGADINLMPI

SEQ ID NO: 59 - protein, 469 aa, Influenza A virus
MNPNQKIITIGSVSLTIATICFLMQIAILVTTVTLHFKQYECSSPPNNQVIPCQPTIIERNITEIVYLTNTTIEKEICPKLVEYRNWSKPQCKITGFAPFSKDNSIRLSAGGDIWVTREPYVSCDPGKCYQFALGQGTTLNNRHSNDTVHDRTPYRTLLMNELGVPFHLGTKQVCIAWSSSSCHDGKAWLHVCVTGHDENATASFIYDGRLVDSIGSWSRNILRTQESECVCINGTCTVVMTDGSASERADTKILFIEEGKVVHISPLSGSAQHVEECSCYPRYPGVRCVCRDNWKGSNRPIVDINVKDYSIISSYVCSGLVGDTPRRNDSSSSSYCQNPNNEKGSHGVKGWAFDDGNDVWMGRTISEELRSGYETFKVIGGWSTPNSKLQINRQVIVDSGNRSGYSGIFSVEGKSCINRCFYVELIRGRKQETKVWWTSNSIVVFCGTSGTYGTGSWPDGADIDLMPI

SEQ ID NO: 60 - protein, 470 aa, Influenza A virus
MNPNQKIITIGSVSLTISTICFFMQVAILITTVTLHFKQYEFNSPPNNQVVLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDRSSSSHCSDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPVP

SEQ ID NO: 61 - protein, 469 aa, Influenza A virus
MNPNQKIITIGSVSLIIATICFLMQIAILVTTITLHFKQHNCDSSPNNHVMFCEPTIIERNKTEIVYLTNTTVEKEICPKPTEYRNWSKPQCNITGFAPFSKDNSIRLSAGGDIWVTREPYVSCDQDKCYQFALGQGTTLNNGHSNDTVHDRTPYRTLLMNELGVPFHLGTRQVCIAWSSSSCHDGKAWLHVCITGDDKNATASLIYNGRLVDSIGSWSKNILRTQESECVCINGTCTVVMTDGSASGKADTKILFIEEGKIIHISTLSGSAQHVEECSCYPRYSGVRCVCRDNWKGSNRPIVDINVKDYSTVSSYICSGLVGDTPRKNDSFSSSNCLDPNNEEGGHGVKGWAFDDGNDLWMGRTISEKSRLGYETFKVVKGWSEPNSKLQSNRQVIVDRGNRSGYSGIFSIEGKNCINRCFYVELIRGRKEETKVLWTSNSIVVFCGTSGTYGTGSWPDGADINLMPI

SEQ ID NO: 62 - protein, 469 aa, Influenza A virus
MNPNQKIITIGSVSLIIATICFLMQVAILVTTVTLHFKQHDCDSSPNNQVMLCEPTIIERNKTEIVYLTNTTVEKEICPKPAEYRNWSKPQCNITGFAPFSKDNSIRLSAGGDIWVTREPYVSCDPDKCYQFALGQGTTLNNGHSNDTVHDRTPYRTLLMNELGVPFHLGTRQVCIAWSSSSCHDGKAWLHVCITGDDKNATASFIYNGRLVDSIGSWSKNILRTQESECVCINGTCTVVMTDGSASGKADTKILFIEEGKIIHISTLSGSAQHVEECSCYPRYPGVRCVCRDNWKGSNRPIVDINVKDYSIVSSYVCSGLVGDTPRKNDSFSSSHCLDPNNEEGGHGVKGWAFDDGNDVWMGRTISEKSRLGYETFKVIKGWSKPNSKLQTNRQVIVGRGNRSGYSGIFSVEGKNCINRCFYVELIRGRKEETKVWWTSNSIVVFCGTSGTYGTGSWPDGADINLMPI

SEQ ID NO: 63 - protein, 469 aa, Influenza A virus
MNPNQKIITIGSVSLIIATICFLMQVAILVTTVKLHFKQYDCNSSSNNQVILCEPTIIERNKTEIVYMTNTTVEKEICPKPAEYRNWSKPQCNIAGFAPFSKDNSIRLSAGGDIWVTREPYVSCDLDKCYQFALGQGTTLNNGHSNDTVHDRTPYRTLLMNELGVPFHLGTRQVCIAWSSSSCHDGKAWLHVCITGDDENATASFIYNGRLVDSIGSWSKNILRTQESECVCINGTCTVVMTDGSASGKADTKILFIMEGKIIHVSKLSGSAQHVEECSCYPRYPGVRCICRDNWKGSNRPIVDINVKDYSIVSSYVCSGLVGDTPRRNDSLSSSHCLDPNNEEGGHGVKGWAFDDGDDVWMGRTINERLRLGYETFKVIKGWSKPNSKLQTNRQIIVEKGNRSGYSGIFSVEGKNCINRCFYVELIRGRKEETRVWWTSNSIVVFCGTSGTYGTGSWPDGADINLMPI

SEQ ID NO: 64 - protein, 469 aa, Influenza A virus
MNPNQKIITIGSVSLTISTICLFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 65 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 66 - protein, 471 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDWPCFSYIFSVEGKSCINRCFYVELIRGRKEETEVLWTSNSIVVFCGTSGTYGTGSWPDGSAPSGHEPVP

SEQ ID NO: 67 - protein, 469 aa, Influenza A virus
MNPNQKIITIGSVSLTISTICFFMQIAILITTVTLHFKQYEFNSPANNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINMKDHSIVSSYVCSGLVGDTPRKSDSSSSSHCLDPNNEKGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMLI

SEQ ID NO: 68 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDRSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 69 - protein, 469 aa, Influenza A virus
MNPNQKIITIGSVSLTISTICFFMQIAILITTVTLHFKQCEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGKLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 70 - protein, 469 aa, Influenza A virus
MNPNQKIITIGSVSLTIATICFLMQIAILVTTVTLHFKQYECNSLPNNQVMLCEPTIIERNITEIVYLTNTTIEKEICPYLSEYRNWSKPQCKITGFAPFSKDNSIRLSAGGDIWVTREPYVSCDPDKCYQFALGQGTTLNNRHSNDTVHDRTPYRTLLMNELGVPFHLGTKQVCIAWSSSSCHDGKAWLHVCVTGHDENATASFIYDGRLVDSIGSWSKKILRTQESECVCINGTCTVVMTDGSASGRADTKILFIEEGKIIHISQLPGSDQHVEECSCYPRYPGVRCVCRDNWKGSNRPIVDINVKDYSIVSSYVCSGLVGDTPRKNDSSSSSHCLNPNNEEGGHGVKGWAFDDGNDVWMGRTISEKFRSGYETFKVIEGWSKPNSKLQINRQVIVDRDNRSGYSGIFSVEGKSCINRCFYVELIRGRKQETEVWWTSNSIVVFCGTSGTYGTGSWPDGADINLMPI

SEQ ID NO: 71 - protein, 469 aa, Influenza A virus
MNPNQKIITIGSVSLTIATICFLMQIAILVTTVTLHFKQYECNSPPNNQVMPCEPTIIERNITEIVYLTNTTIEREICPKLVEYRNWSKPQCRVTGFAPFSKDNSIRLSAGGDIWVTREPYVSCDPGKCYQFALGQGTTLNNRHSNDTVHDRTPYRTLLMNELGVPFHLGTKQVCIAWSSSSCHDGKAWLHVCVTGHDENATASFIYDGRLVDSIVSWSKNILRTQESECVCINGTCTVVMTDGSASERADTKILFIEEGKIVHISPLSGSAQHVEECSCYPRYPGVRCVCRDNWKGSNRPIVDINVKDYSIVSSYVCSGLVGDTPRKNDSSSSSYCRNPNNEKGSHGVKGWAFDDGNDVWMGRTIGEELRSGYETFKVIGGWSTPNSKLQTNRQVIVDRGNRSGYSGIFSVEGKSCINRCFYVELIRGRKQETKVWWTSNSIVVFCGTSGTYGTGSWPDGADINLMPI

SEQ ID NO: 72 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLIDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 73 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 74 - protein, 469 aa, Influenza A virus
MNPNQKIITIGSVSLTIAAICLLLQIAALVTTITLHFKQHECNFPPNNQVMLCEPTIIERNITEIVYLTNTTIEKEICPKMAEYRNWSKPQCKITGFAPFSKDNSIRLSAGGDIWVTREPYVSCDPDKCYQFALGQGTTLNNKHSNDTVHDRTPYRTLLMNELGVPFHLGTKQVCIAWSSSSCHDGKAWLHVCVTGHDQNATASFIYDGRLVDSIGSWSKNILRTQESECVCINGTCAVVMTDGSASGKADTKILFIEEGKIVHISQLSGSAQHVEECSCYPRYPGVRCVCRDNWKGSNRPIVDINIKDHSIVSSYMCSGLVGDTPRKNDSSSSSHCLNPNNEEGGHGVKGWAFDDENDVWMGRTISEKLRFGYETFRVIDGWTKANSKSQINRQVIVDKDNRSGYSGIFSVEGKGCINRCFYVELIRGRKQETEVWWTSNSIVVFCGTSGTYGTGSWPDGADINLMPI

SEQ ID NO: 75 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 76 - protein, 469 aa, Influenza A virus
MNPNQKIITIGSVSLTISTICFFMQIAILITTVTLHFKQYEFSSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPFRTLLMNELGVPFHLGTKQVCIAWSSSSCHDGKAWLHVCITGDDKNATASFIYNGRLVDSVVSWSKEILRTQESECVCINGTCTVVMTDGSASGKAVTKILFIEEGKIVHTSTLSGSAQHVEECSCYPRYPGVRCVCRDNWKGSNRPIVDINIKDHSIVSGYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 77 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMHI

SEQ ID NO: 78 - protein, 469 aa, Influenza A virus
MNPNQKIITIGSVSLTISTICFFMQIAILITTVTLHFKQYEFNPPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMSELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKNHSIASSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMLI

SEQ ID NO: 79 - protein, 469 aa, Influenza A virus
  VARIANT 59: note = Any amino acid
MNPNQKIITIGSVSLTISTICFFMQIAILITTVTLHFKQYEFNSPPNNQVMLCEPTIIXRNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 80 - protein, 470 aa, Influenza A virus
MNPNQKIITIGSVSLTISTICFFMQIAILITTVTLHFKQYEFNSPPNNQVMLCEPTIIERNITEIVYLTNTTMEMGRNNQGEDRIWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGAAPSGHEPVP

SEQ ID NO: 81 - protein, 469 aa, Influenza A virus
MNPNQKIITIGSVSLIIATVCFLMQIAILVTTVTLHFKQHECDSPSSNQVMPCEPIIIERNITEIVYLNNTTIEKETCPKLVEYRNWSKPQCKITGFAPFSKDNSIRLSAGGDIWVTREPYVSCDPGKCYQFALGQGTTLDNKHSNDTIHDRIPHRTLLMNELGVPFHLGTRQVCIAWSSSSCHDGKAWLHVCVTGDDKNATASFIYDGRLVDSIGSWSQNILRTQESECVCINGTCTVVMTDGNASGRADTRILFIEEGKIVHISPLSGSAQHVEECSCYPRYPDVRCICRDNWKGSNRPIVDINMKDYSIDSSYVCSGLVGDTPRNDDRSSKSNCRNPNNERGNHGVKGWAFDNGNDVWMGRTISKDLRSGYETFKVIGGWSTPNSKSQINRQVIVDSDNRSGYSGIFSVEGKSCINRCFYVELIRGREQETRVWWTSNSIVVFCGTSGTYGTGSWPDGANINFMPI

SEQ ID NO: 82 - protein, 469 aa, Influenza A virus
MNPNQKIITIGSVSLTISTICFFMQIAILVTTVTLHFKQYECNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNGHSNDIVHDRTPYRTLLMNELGVPFHLGTKQVCIAWSSSSCHDGKAWLHVCVTGDDENATASFIYNGRLVDSIGSWSKKFFRTQESECVCIHGTCTVVMTDGSASRKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 83 - protein, 469 aa, Influenza A virus
MNPNQKIITIGSVSLTIATICFLMQIAILVTTVTLHFKQHECNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNGHSNDTVRDRTPYRTLLMNELGVPFHLGTRQVCIAWSSSSCHDGKAWLHVCVTGDDKNATASFIYNGRLVDSIGSWSKKIFRTQKFECVCIHWTCTVVMTDGSASGKADTKILFIEEGKIVHISTLSGSAQHVEECSCYPRYPGVRCVCRDNWKGSNRPIVDINVKDYSIVSSYVCSGLVGDTPRKNDSFSSSHCLDPNNEEGGHGVKGWAFDDGNDVWMGRTISEKLRSGYETFKVIEGWSKPNSKLQINRQVIVERGNRSGYSGIFSVEGKSCINRCFYVELIRGRKEETEVWWTSNSIVVFCGTSGTYGTGSWPDGADINLMPI

SEQ ID NO: 84 - protein, 470 aa, Influenza A virus
MNPNQKIITIGSVSLTISTICFFMQIAILITTVTLHFKQYEFNSPPNNQVMLCEPTIIERNITEIVYLTNTTIEREMCPKPAEFRNWSKPQCGITGFAPFSKDNSIRLSAGGDIWVTREPYVSCDPDKCYQFALGQGTTLDNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMSLF

SEQ ID NO: 85 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADINLMPI

SEQ ID NO: 86 - protein, 469 aa, Influenza A virus
MNPNQKIITIGSVSLTISTICFFMQIAILITTVTLHFKQYEFNPPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 87 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVHDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 88 - protein, 469 aa, Influenza A virus
MNPNQKIITIGSVSLTISTICFFMQIAILITTVTLHFKQYEFNSPANNQVMLCEPTTIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEKGGHGVKGWAFDDGNDVWMGRTINETSRLGYETFKVIEGWSNPKSKLQTNRQVIVDRGDRSGYSGIFSVESKSCINRCFYVELIRGRKEETEVLWTSNSIVVFCGTSGTYGTGSWPDGADLNLMHI

SEQ ID NO: 89 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCAVVMTDGSASGKADTKILFIEEGKIVHTSKLSGSAQHVEECSCYPRYPGIRCVCRDNWKGSNRPIVDINIKDHSIVSNYVCSGLVGDTPRKNDSSSSSHCLDPNNEEGGHGVKGWAFDDGNDVWMGRTINEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADLNLMPI

SEQ ID NO: 90 - protein, 468 aa, Influenza A virus
MNPNQKIITIGSVSLTISTICFFMQIAILITTVTLHFKQYEFNSPPNNQVMLCEPTIIERNITEVVYLTNTTIEKEICPKPAEYRNWSKPQCQITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSQNILRTQESECVCINGTCTVVMTDGSASGRADTRILFIEEGKIVHISPLSGSAQHVEECSCYPRYPGVRCVCRDNWKGSNRPIVDINMEDHSIVSSYVCSGLVGDTPRKNDSSSSSHCLNPNNERGHGVKGWAFDDGNDVWMGRTINEDSRLGYETFKVIGGWSTPNSKSQINRQVIVDSDNRSGYSGIFSVEGKSCINRCFYVELIRGRKQETRVWWTSNSIVVFCGTSGTYGTGSWPDGADLNLMHI

SEQ ID NO: 91 - protein, 468 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCQITGFAPFSKDNSIRLSAGGDIWVTREPYVSCDPDKCYQFALGQGTTINNVHSNNTARDRTPYRTLLMNELGVPFHLGTKQVCIAWSSSSCHDGKAWLHVCITGDDKNATASFIYDGRLVDSIGSWSQNILRTQESECVCINGTCTVVMTDGNATGKADTKILFIEEGKIVHTSKLSGSAQHVEECSCYPRYPGVRCVCRDNWKGSNRPIVDINMEDHSIVSSYVCSGLVGDTPRNDDRSSNSNCLNPNNERGHGVKGWAFDDGNDVWMGRTINEDSRLGYETFKVVEGWSNPKSKLQINRQVIVDRGDRSGYSGIFSVEGKSCINRCFYVELIRGRKQETRVWWTSNSIVVFCGTSGTYGTGSWPDGADLNLMHI

SEQ ID NO: 92 - protein, 468 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCQITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSQNILRTQESECVCINGTCTVVMTDGSASGRADTRILFIEEGKIVHTSKLSGSAQHVEECSCYPRYPGVRCICRDNWKGSNRPVVDINMEDYSIDSSYVCSGLVGDTPRKNDSSSSSHCLNPNNERGHGVKGWAFDDGNDVWMGRTINEDSRFGYETFKVVEGWSNPKSKLQINRQVIVDRDNRSGYSGIFSVEGKSCINRCFYVELIRGRKQETRVWWTSNSIVVFCGTSGTYGTGSWPDGADLNLMHI

SEQ ID NO: 93 - protein, 469 aa, Influenza A virus
MNPNQKIITIGSVSLTISTICFFMQIAILITTVTLHFKQYEFNSPPNNQVMLCEPTIIERNITEIVYLNNTTIEKEICPKVVEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYNGKLVDSVVSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGADLNLMHI

SEQ ID NO: 94 - protein, 469 aa, Influenza A virus
MNPNQKIITIGSVSLTISTICFFMQIAILITTVTLHFKQYEFNPPPNNQVMLCEPTIIERNITEIVYLTNTTIEKEICPKLAEYREWSKPQCNITGFAPFSKDNSIRLSAGGDIWVTREPYVSCDPDKCYQFALGQGTTLNNVHSNDIVRDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWRGSNRPIVDINMKDYSIVSSYVCSGLVGDTPRKNDSSSSGHCLNPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 95 - protein, 469 aa, Influenza A virus
MYPIKKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHGRTPYRTLLMNELGVPFHLGTKQVCIAWSSSSCHDGKAWLHVCVTGDDKNATASFIYNGRLVDSVVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSRYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRNQETEVLWTSNSIVVFCGTSGTYGTGSWPDEGNINHMPI

SEQ ID NO: 96 - protein, 469 aa, Influenza A virus
MNPNQKIITIGSVSLTISTICFLMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGKLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDRSSSSHCLDPNNEEGGHGVKGWAFDDGNDVWMGRTINETSRLGYETFKVIEGWSNPKSKLQINRQVIVDRGNRSGYSGIFSVEGKSCINRCFYVELIRGRKEETEVLWTSNSIVVFCGTSGTYGTGSWPDGADLNLMPI

SEQ ID NO: 97 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIKEGKIVHTSTLSGSAQHVEECSCYPRYPGVRCVCRDNWKGSNRPIVDINIKDHSIVSSYVCSGLVGDTPRKTDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 98 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKKILRTQESECVCINRTCIVVMTDGSASGKADTKILFIEEGKIVHTSTLSGSAQHVEECSCYPRYPDVRCVCRDNWKGSNRPIVDINIKDHSIVSRYVCSGLVGDTPRKNDSSSSSHCLDPNNEEGGHGVKGWAFDDGNDVWMGRTISETSRLGYETFKVIEGWSNSKSKLQINRQVIVDRGNRSGYSGIFSVEGKSCINRCFYVELIRGRKEETEVLWTSNSIVVFCGTSGTYGTGSWPDGADLNLMPI

SEQ ID NO: 99 - protein, 469 aa, Influenza A virus
MNPNQKIITIGSVSLTIATICFLVQIAILVTTVTLHFKQYECNSPPNNQVMLCEPTIIERNITEIVYLTNTTIEKEICPKLAEYRNWSKPQCKITGFAPFSKDNSIRLSAGGDIWVTREPYVSCDPDKCYQFALGQGTTLNNRHSNDTVHDRTPYRTLLMNELGVPFHLGTKQVCIAWSSSSCHDGKAWLHVCVTGDDENATASFIYDGRLVDSIGSWSKKILRTQESECVCINGTCTVVMTDGSASGRADTKILFIEEGKIVHTSKLSGSAQHVEECSCYPRYPGVRCVCRDNWKGSNRPIVDINVKDYSIVSSYVCSGLVGDTPRKNDSSSSSHCLDPNNEEGGHGVKGWAFDDGDDVWMGRTISEKSRSGYETFKVIEGWSKPNSKLQINRQVIVERGNMSGYSGIFSVEGKSCINRCFYVELIRGRKQETEVWWTSNSIVVFCGTSGTYGTGSWPDGADINLMPI

SEQ ID NO: 100 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLYPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 101 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCAITGFAPFSKDNSIRLSAGGDIWVTREPYVSCDPDKCYQFALGQGTTLNNVHSNNIVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKVVHTSTLSGSAQHVEECSCYPRYPGVRCVCRDNWKGSNRPIVDINIKDRSVVSSYVCSGLVGDTPRKNDSSSSSHCLDPNNEEGGNGVKGWAFDDGTDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 102 - protein, 469 aa, Influenza A virus
MNPNQKIITIGSVSLTIATICFLMQIAILVTTVTLHFKQYECSSPPNNQVMPCEPIIIERNITEIVYLTNTTIEKEICPKLVEYRNWSKPQCKITGFAPFSKDNSIRLSAGGGIWVTREPYVSCDPGKCYQFALGQGTTLDNKHSNDTIHDRTPYRTLLMNELGVPFHLGTRQVCIAWSSSSCHDGKAWLHVCVTGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSDNRSGYSGIFSVEGKSCINRCFYVELIRGREQETRVWWTSNSIVVFCGTSGTYGTGSWPDGADINLMPI

SEQ ID NO: 103 - protein, 469 aa, Influenza A virus
MNPNQKIITIGSVSLTISTICFFMQIAILVTTVTLHFKQYECNSPPNNQVMLCEPTIIERNITEIVYLTNTTIEKEIYPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSFSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 104 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPYSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 105 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHRLDPNNEEGGHGVKGWAFDDGNDVWMGRTINETSRLGYETFKVIEGWSNPKSKLQINRQVIVDKGNRSGYSGIFSVEGKSCINRCFYVELIRGRKEETEVLWTSNSIVVFCGTSGTYGTGSWPDGADLNLMPI

SEQ ID NO: 106 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGGIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNASGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 107 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPNKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINMKDHSIVSSYVCSGLVGDTPRKNDSSSSSHCFDPNDEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGAYGTGSWPDGADINLMPI

SEQ ID NO: 108 - protein, 469 aa, Influenza A virus
MNPNQKIITIGSVSLTISTICFFMQIAILITTVTLHFKQYEFNSPPNNQVMLCEPTIVERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPFRTLLMNELGVPFHLGTKQVCIAWSSSSCHDGKAWLHVCITGDDKNATASFIYNGRLVDSVVSWSKEILRTQESECVCINGTCTVVMTDGSASGKAVTKILFIEEGKIVHTSTLSGSAQHVEECSCYPRYPGVRCVCRDNWKGSNRPIVDINIKDHSIVSGYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 109 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGGIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 110 - protein, 469 aa, Influenza A virus
MNPNQKIITIGSVSLTISTICFFMQIAILITTVTLHFKQYEFNSPPNNQVMLCEPTIIERNITEIVYLTNTTIEKEIYPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSGHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 111 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCMAWSSSSCHDGKAWLHVCVTGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKSDSSSSSHCLNPNNEEGGHGVKGWAFDDGNDVWMGRTINETSRLGYETFKVVEGWSNSKSKLQINRQVIVDRGDRSGYSGIFSVEGKSCINRCFYVELIRGRKEETEVLWTSNSIVVFCGTSGTYGTGSWPDGADLNLMHI

SEQ ID NO: 112 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNIVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSSPDGADINLMPI

SEQ ID NO: 113 - protein, 469 aa, Influenza A virus
MNPNQKIITIGSVSLTISTICFFMQIAILITTVTLHFKQYEFNSPPNNQVMLCESTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPFRTLLMNELGVPFHLGTKQVCIAWSSSSCHDGKAWLHVCITGDDKNATASFIYNGRLVDSVVSWSKEILRTQESECVCINGTCTVVMTDGSASGKAVTKILFIEEGKIVHTSTLSGSAQHVEECSCYPRYPGVRCVCRDNWKGSNRPIVDINIKDHSVVSGYVCSGLVGDTPRKNDSSSSSHCLDPNNEEGGHGVKGWAFDDGDDVWMGRTINETSRLGYETFKVIEGWSNPKSKLQINRQVIVDKGDRSGYSGIFSVEGKSCINRCFYVELIRGRKEETEVLWTSNSIVVFCGTSGTYGTGSWPDGADLNLMPI

SEQ ID NO: 114 - protein, 469 aa, Influenza A virus
MNPNQKIITIGSVSLTIATICFLMQIAILVTTVTLHFKQYECSSSPNNQVIPCQPTIIERNITEIVYLTNTTIEKEICPKLVEYRNWSKPQCKITGFAPFSKDNSIRLSAGGDIWVTREPYVSCDPGKCYQFALGQGTTLNNRHSNDTVHDRTPYRTLLMNELGVPFHLGTKQVCIAWSSSSCHDGKAWLHVCVTGHDENATASFIYDGRLVDSIGSWSKNILRTQESECVCINGTCTVVMTDGSASEKADTKILFIEEGKVVHISPLSGSAQHVEECSCYPRYPGVRCVCRDNWKGSNRPIVDINVKNYSIVSSYVCSGLVGDTPRKNDSSSSSYCRNPNNEKGSHGVKGWAFDDGNDVWMGRTISEELRSGYETFKVIGGWSTPNSKLQINRQVIVDSGNRSGYSGIFSVEGKSCINRCFYVELIRGRKQETKVWWTSNSLVVFCGTSGTYGTGSWPDGADINLMPI

SEQ ID NO: 115 - protein, 469 aa, Influenza A virus
MNPNQKIITIGSVSLTIATICFLMQIAILVTTVTLHFKQYECDSPANNQIMPCEPTIIERNITEIVYLTNITIEKEICPKLVEYRNWSKPQCKITGFAPFSKDNSIRLSAGGDIWVTREPYVSCDPSRCYQFALGQGTTLDNKHSNDTIHDRTPHRTLLMNELGVPFHLGTRQVCIAWSSSSCHDGKAWLHVCVTGYDKNATASFIYDGRLVDSIGSWSQNILRTQESECVCINGTCTVVMTDGSASGRADTKILFIEEGKIVHISPLSGSAQHVEECSCYPRYPGVRCICRDNWKGSNRPVVDINVKDYSIDSSYVCSGLVGDTPRKNDRSSNSYCRNPNNEKGNHGVKGWAFDDGNDVWMGRTISEDSRSGYETFKVIGGWSTPNSKLQINRQVIVDSDNRSGYSGIFSVEGKSCINRCFYVELIRGREQETRVWWTSNSIVVFCGTSGTYGAGSWPDGADINLMPI

SEQ ID NO: 116 - protein, 493 aa, synthetic construct
MPMGSLQPLATLYLLGMLVASVLSAHHHHHHGSGSLVPRGSPSRSIINETADDIVYRLTVIIDDRYESLKNLITLRADRLEMIINDNVSTILASIGSGKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLIDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 117 - protein, 423 aa, synthetic construct
MPMGSLQPLATLYLLGMLVASVLSAGSGKEICPKLVEYRNWSKPQCKITGFAPFSKDNSIRLSAGGGIWVTREPYVSCDPGKCYQFALGQGTTLDNKHSNDTIHDRTPYRTLLMNELGVPFHLGTRQVCIAWSSSSCHDGKAWLHVCVTGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSDNRSGYSGIFSVEGKSCINRCFYVELIRGREQETRVWWTSNSIVVFCGTSGTYGTGSWPDGADINLMPI

SEQ ID NO: 118 - protein, 423 aa, synthetic construct
MPMGSLQPLATLYLLGMLVASVLSAGSGKEIYPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSFSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 119 - protein, 423 aa, synthetic construct
MPMGSLQPLATLYLLGMLVASVLSAGSGKEICPKPAEYRNWSKPQCGITGFAPYSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 120 - protein, 423 aa, synthetic construct
MPMGSLQPLATLYLLGMLVASVLSAGSGKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHRLDPNNEEGGHGVKGWAFDDGNDVWMGRTINETSRLGYETFKVIEGWSNPKSKLQINRQVIVDKGNRSGYSGIFSVEGKSCINRCFYVELIRGRKEETEVLWTSNSIVVFCGTSGTYGTGSWPDGADLNLMPI

SEQ ID NO: 121 - protein, 423 aa, synthetic construct
MPMGSLQPLATLYLLGMLVASVLSAGSGKEICPKPAEYRNWSKPQCGITGFAPFSKDNSIRLSAGGGIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNASGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 122 - protein, 423 aa, synthetic construct
MPMGSLQPLATLYLLGMLVASVLSAGSGKEICPKLAEYRNWSKPQCDITGFAPFSKDNSIRLSAGGDIWVTREPYVSCDPNKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINMKDHSIVSSYVCSGLVGDTPRKNDSSSSSHCFDPNDEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGAYGTGSWPDGADINLMPI

SEQ ID NO: 123 - protein, 423 aa, synthetic construct
MPMGSLQPLATLYLLGMLVASVLSAGSGKEICPKPAEYRNWSKPQCGITGFAPFSKDNSIRLSAGGDIWVTREPYVSCDPDKCYQFALGQGTTLNNVHSNNTVRDRTPFRTLLMNELGVPFHLGTKQVCIAWSSSSCHDGKAWLHVCITGDDKNATASFIYNGRLVDSVVSWSKEILRTQESECVCINGTCTVVMTDGSASGKAVTKILFIEEGKIVHTSTLSGSAQHVEECSCYPRYPGVRCVCRDNWKGSNRPIVDINIKDHSIVSGYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 124 - protein, 423 aa, synthetic construct
MPMGSLQPLATLYLLGMLVASVLSAGSGKEICPKPAEYRNWSKPQCGITGFAPFSKDNSIRLSAGGGIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 125 - protein, 423 aa, synthetic construct
MPMGSLQPLATLYLLGMLVASVLSAGSGKEIYPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSGHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 126 - protein, 423 aa, synthetic construct
MPMGSLQPLATLYLLGMLVASVLSAGSGKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCMAWSSSSCHDGKAWLHVCVTGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKSDSSSSSHCLNPNNEEGGHGVKGWAFDDGNDVWMGRTINETSRLGYETFKVVEGWSNSKSKLQINRQVIVDRGDRSGYSGIFSVEGKSCINRCFYVELIRGRKEETEVLWTSNSIVVFCGTSGTYGTGSWPDGADLNLMHI

SEQ ID NO: 127 - protein, 423 aa, synthetic construct
MPMGSLQPLATLYLLGMLVASVLSAGSGKEICPKLAEYRNWSKPQCDITGFAPFSKDNSIRLSAGGDIWVTREPYVSCDPDKCYQFALGQGTTLNNVHSNNIVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSSPDGADINLMPI

SEQ ID NO: 128 - protein, 423 aa, synthetic construct
MPMGSLQPLATLYLLGMLVASVLSAGSGKEICPKPAEYRNWSKPQCGITGFAPFSKDNSIRLSAGGDIWVTREPYVSCDPDKCYQFALGQGTTLNNVHSNNTVRDRTPFRTLLMNELGVPFHLGTKQVCIAWSSSSCHDGKAWLHVCITGDDKNATASFIYNGRLVDSVVSWSKEILRTQESECVCINGTCTVVMTDGSASGKAVTKILFIEEGKIVHTSTLSGSAQHVEECSCYPRYPGVRCVCRDNWKGSNRPIVDINIKDHSVVSGYVCSGLVGDTPRKNDSSSSSHCLDPNNEEGGHGVKGWAFDDGDDVWMGRTINETSRLGYETFKVIEGWSNPKSKLQINRQVIVDKGDRSGYSGIFSVEGKSCINRCFYVELIRGRKEETEVLWTSNSIVVFCGTSGTYGTGSWPDGADLNLMPI

SEQ ID NO: 129 - protein, 423 aa, synthetic construct
MPMGSLQPLATLYLLGMLVASVLSAGSGKEICPKLVEYRNWSKPQCKITGFAPFSKDNSIRLSAGGDIWVTREPYVSCDPGKCYQFALGQGTTLNNRHSNDTVHDRTPYRTLLMNELGVPFHLGTKQVCIAWSSSSCHDGKAWLHVCVTGHDENATASFIYDGRLVDSIGSWSKNILRTQESECVCINGTCTVVMTDGSASEKADTKILFIEEGKVVHISPLSGSAQHVEECSCYPRYPGVRCVCRDNWKGSNRPIVDINVKNYSIVSSYVCSGLVGDTPRKNDSSSSSYCRNPNNEKGSHGVKGWAFDDGNDVWMGRTISEELRSGYETFKVIGGWSTPNSKLQINRQVIVDSGNRSGYSGIFSVEGKSCINRCFYVELIRGRKQETKVWWTSNSLVVFCGTSGTYGTGSWPDGADINLMPI

SEQ ID NO: 130 - protein, 423 aa, synthetic construct
MPMGSLQPLATLYLLGMLVASVLSAGSGKEICPKLVEYRNWSKPQCKITGFAPFSKDNSIRLSAGGDIWVTREPYVSCDPSRCYQFALGQGTTLDNKHSNDTIHDRTPHRTLLMNELGVPFHLGTRQVCIAWSSSSCHDGKAWLHVCVTGYDKNATASFIYDGRLVDSIGSWSQNILRTQESECVCINGTCTVVMTDGSASGRADTKILFIEEGKIVHISPLSGSAQHVEECSCYPRYPGVRCICRDNWKGSNRPVVDINVKDYSIDSSYVCSGLVGDTPRKNDRSSNSYCRNPNNEKGNHGVKGWAFDDGNDVWMGRTISEDSRSGYETFKVIGGWSTPNSKLQINRQVIVDSDNRSGYSGIFSVEGKSCINRCFYVELIRGREQETRVWWTSNSIVVFCGTSGTYGAGSWPDGADINLMPI

SEQ ID NO: 131 - protein, 423 aa, synthetic construct
MPMGSLQPLATLYLLGMLVASVLSAGSGKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 132 - protein, 24 aa, synthetic construct
MPMGSLQPLATLYLLGMLVASVLS

SEQ ID NO: 133 - protein, 24 aa, synthetic construct
MPMGSLQPLATLYLLGMLVASCLG

SEQ ID NO: 134 - protein, 4 aa, synthetic construct
AGSG

SEQ ID NO: 135 - protein, 6 aa, synthetic construct
HHHHHH

SEQ ID NO: 136 - protein, 36 aa, Influenza A virus
RNITEIVYLTNTTIEKEICPKLAEYRNWSKPQCDIT

SEQ ID NO: 137 - protein, 36 aa, Influenza A virus
RNITEIVYLTNTTIEKEICPKPAEYRNWSKPQCGIT

SEQ ID NO: 138 - protein, 36 aa, Influenza A virus
RNITEIVYLTNTTIEREICPKPAEYRNWSKPQCGIT